(19) United States
(12) Patent Application Publication
(10) Pub. No.: US 2026/0014345 A1
(43) Pub. Date: Jan. 15, 2026

(54) SYSTEM AND METHOD FOR INDUCING A SENSATION OF MOTION IN A SUBJECT USING AUDITORY STIMULI TO FACILITATE SLEEP

(71) Applicant: Neurolight, Inc., Pomona, NY (US)

(72) Inventor: Alexander I. Poltorak, Pomona, NY (US)

(21) Appl. No.: 19/268,995

(22) Filed: Jul. 14, 2025

Related U.S. Application Data
(60) Provisional application No. 63/670,627, filed on Jul. 12, 2024.

(51) Int. Cl. A61M 21/00

(52) U.S. Cl. CPC A61M 21/00; A61M 2021/0022; A61M 2021/0027; A61M 2021/005; A61M 2205/3303; A61M 2230/08

(57) ABSTRACT

A method for inducing a sensation of motion in a subject, comprising defining a vibrational stimulation pattern for a subject; generating a set of electrical signals corresponding to the vibrational stimulation pattern; and transducing the set of electrical signals into a vibrations corresponding to the set of electrical signals with a plurality of electromechanical transducers, wherein the vibrations are adapted to induce the sensation of motion in the subject.

### CROSS REFERENCE TO RELATED APPLICATIONS

The present application is a non-provisional of, and claims benefit of priority under 35 U.S.C. § 119(e) from, U.S. Provisional Patent Application No. 63/670,627, the entirety of which is expressly incorporated herein by reference.

### FIELD OF THE INVENTION

The present invention generally relates to sleep aids, and more particularly to systems and methods for facilitating sleep through sensory brain stimulation, specifically utilizing auditory stimuli to induce a sensation of motion in a subject.

### INCORPORATION BY REFERENCE AND INTERPRETATION OF LANGUAGE

Citation or identification of any reference herein, in any section of this application, shall not be construed as an admission that such reference is necessarily available as prior art to the present application. The disclosures of each reference disclosed herein, whether U.S. or foreign patent literature, or non-patent literature, are hereby incorporated by reference in their entirety in this application, and shall be treated as if the entirety thereof forms a part of this application.

All cited or identified references are provided for their disclosure of technologies to enable practice of the present invention, to provide basis for claim language, and to make clear applicant's possession of the invention with respect to the various aggregates, combinations, and subcombinations of the respective disclosures or portions thereof (within a particular reference or across multiple references). The citation of references is intended to be part of the disclosure of the invention, and not merely supplementary background information. The incorporation by reference does not extend to teachings which are inconsistent with the invention as expressly described herein (which may be treated as counter examples), and is evidence of a proper interpretation by persons of ordinary skill in the art of the terms, phrase and concepts discussed herein, without being limiting as the sole interpretation available.

The present specification is not to be interpreted by recourse to lay dictionaries in preference to field-specific dictionaries or usage. Where a conflict of interpretation exists, the hierarchy of resolution shall be the express specification, references cited for propositions, incorporated references, the inventors' prior publications relating to the field, academic literature in the field, commercial literature in the field, field-specific dictionaries, lay literature in the field, general purpose dictionaries, and common understanding. Where the issue of interpretation of claim amendments arises, the hierarchy is modified to include arguments made during the prosecution and accepted without retained recourse.

### BACKGROUND OF THE INVENTION

In the modern world, an increasing number of individuals suffer from sleep-related issues. Factors such as stress, anxiety, irregular work schedules, and excessive use of electronic devices contribute to difficulties in falling asleep and maintaining sleep. This has led to a growing interest in methods and devices that can assist individuals in achieving better sleep quality.

Existing solutions for sleep induction include pharmacological interventions, which often come with side effects and the risk of dependency, and non-pharmacological methods such as cognitive-behavioral therapy for insomnia (CBT-I), which may require significant time and commitment. Additionally, there are various sleep aids like noise machines, sleep sounds, and calming music, but these often lack the personalized approach needed to address the unique sleep patterns of different individuals.

See, U.S. Patent and Pub. Pat. Application Nos. 20240233288; U.S. Pat. Nos. 12,029,573; 12,029,714; 20240165370; 20240156407; 20240149010; 20240103686; 20240094319; 20240090677; 20240051815; 20240033465; 20230404466; 20230390571; 20230330861; 20230256192; 20230200623; 20230143155; 20230106753; 20230041557; 20230000269; 20220408147; 20220387749; 20220386018; 20220354356; 20220313945; 20220280747; 20220270566; 20220218980; 20220167930; 20220160994; 20220133156; 20220079514; 20220061766; U.S. Pat. No. 11,247,032; 20210401184; U.S. Pat. No. 11,207,490; 20210386964; 20210361165; 20210361172; 20210353904; U.S. Pat. No. 11,170,102; 20210338973; 20210318660; 20210299398; U.S. Pat. No. 11,116,973; 20210257080; 20210236806; 20210228150; 20210205574; 20210204720; 20210178112; 20210170137; 20210114603; 20210085243; U.S. Pat. Nos. 10,945,953; 20210045678; 20210046312; 20210038459; 20210008335; 20200397349; 20200383580; 20200368491; 20200359954; 20200360653; 20200330322; 20200281525; 20200286505; 20200155076; 20200135047; 20200129727; 20200093426; 20200086078; 20200070712; 20190387998; 20190374742; U.S. Pat. No. 10,479,235; 20190328996; 20190321585; 20190321583; 20190282779; 20190282780; 20190247611; 20190247010; 20190231256; 20190224443; 20190223747; 20190209806; 20190200872; 20190192069; 20190182329; 20190143116; 20190143073; 20190105098; 20190083003; U.S. Pat. No. 10,232,139; 20190021641; 20180344968; 20180333558; 20180262583; 20180255335; 20180250494; 20180193588; 20180193589; 20180197158; 20180116415; 20180110461; 20180078734; 20180075091; 20180027988; 20180018328; 20170351750; 20170312476; 20170281899; 20170281119; 20170289665; 20170252533; 20170245555; 20170231560; 20170173299; U.S. Pat. No. 9,662,391; 20170143935; 20170149945; 20170095199; 20170094385; 20170053068; 20170028166; 20170000970; 20160331306; U.S. Pat. No. 9,449,150; 20160228674; 20160220783; 20160205489; 20160174728; 20160151603; 20160144173; 20160082222; 20150374951; 20150367097; 20150359773; 20150306391; 20150297109; 20150154881; 20150087894; 20150045654; 20150045608; 20140343354; 20140316192; 20140307878; 20140275741; 20140253032; 20140236589; 20130301845; 20130296636; 20130283530; 20130123572; 20130079584; U.S. Pat. No. 8,391,506; 20120296156; 20120248832; 20120131747; 20120100922; 20120041343; 20110246508; U.S. Pat. No. 8,012,189; 20110185499; 20110172725; 20110166937; 20110145009; 20110034756; 20110014847; 20100250248; 20100192945; U.S. Pat. Nos. 7,749,155; 20100153313; 20100153584; 20100145854; 20100145410; 20100141399; 20100130811; 20100131790; 20100125561; 20100102971; 20100100036; 20100081434; 20100081860; 20100081861; 20100063368; 20100048984; U.S. Pat. No. 7,668,754; 20100041958; 20100041964; 20100042671; 20100032481; 20100030089; 20100022820; 20100009324; 20100004762; 20090321523; 20090292676; 20090287039; 20090267758; 20090270687; 20090270692; 20090270693; 20090270694; 20090271008; 20090271010; 20090271122; 20090260630; 20090216438; 20090209278; 20090158920; 20090132072; 20090122700; 20090095299; 20090089928; 20090093216; 20090086676; 20090088089; 20090089569; 20090083826; 20090059897; 20090007143; 20080317240; 20080296380; 20080299951; 20080300109; 20080208620; 20080209046; 20080135437; 20080133268; 20080123332; 20080114577; 20080082272; 20080082368; 20080075261; 20080070697; 20080052114; 20080046395; 20080033763; 20080033762; 20080004909; 20080005057; 20080005695; 20070287740; 20070225322; 20070191100; 20070162569; 20070154046; 20070150512; 20070136222; 20070099602; 20070100480; 20070101274; 20070083079; 20070037566; 20070022372; 20070015494; 20070002011; 20070004969; 20070005363; 20070005988; 20060293874; 20060293893; 20060294582; 20060281543; 20060267940; 20060252761; 20060216365; 20060150238; 20060105671; 20060084659; 20060041632; 20060019224; 20060020161; 20050283039; 20050278778; 20050266868; 20050248962; 20050237582; U.S. Pat. No. 6,954,668; 20050139679; 20050143617; 20050137987; 20040143296; U.S. Pat. No. 6,760,850; 20040042166; 20040002635; U.S. Pat. Nos. 6,654,896; 6,647,512; 6,615,197; 6,515,103; 6,503,188; 6,463,545; 20020124197; 20020115905; U.S. Pat. No. 6,399,574; 20020040018; U.S. Pat. Nos. 6,356,965; 6,277,825; 6,253,319; 6,172,041; 5,954,629; 5,853,005; 5,676,633; 5,551,879; 5,449,683; 5,377,841; 5,357,642; 5,356,368; 5,351,819; 5,318,503; 5,226,021; 5,197,941; 5,063,912; 4,388,918; 4,371,815; 4,370,602; 4,282,864; 4,227,516; 3,993,043; 3,898,383; 3,884,218; 3,762,396; 3,712,292; 3,611,151; 3,255,753; 3,031,686; and 2,644,153.

A soft and gentle movement, such as rocking or swaying, often helps a subject to fall asleep. Mothers rock their babies to sleep in a crib while singing lullabies. Recent studies show that it is the rocking rather than singing that helps infants to fall asleep.

Rocking engages a subject's vestibular system, which is responsible for balance, spatial orientation, and the perception of motion. The vestibular system is located in the inner ear and consists of structures called the semicircular canals and the otolith organs. When a subject is rocked, the vestibular system is stimulated in the following way:

The semicircular canals are three fluid-filled tubes located in each inner ear. These canals are oriented in different directions (horizontal, vertical, and diagonal) and are responsible for detecting rotational movements of the head.

Rocking a baby cradle for example produces a back-and-forth or side-to-side motion. As the cradle moves, the fluid in the semicircular canals also moves, causing tiny hair cells within the canals to bend. This bending of hair cells sends signals to the brain indicating the direction and speed of the rocking motion. The rocking motion produces similar effects on an adult.

The otolith organs, consisting of the utricle and saccule, are also part of the vestibular system. They are responsible for detecting linear movements and changes in head position with respect to gravity. See:
Angelaki, Dora E., and Kathleen E. Cullen. “Vestibular system: the many facets of a multimodal sense.” Annu. Rev. Neurosci. 31, no. 1 (2008): 125-150.
Barmack, Neal H. “Central vestibular system: vestibular nuclei and posterior cerebellum.” Brain research bulletin 60, no. 5-6 (2003): 511-541.
Casale, Jarett, Tivon Browne, I. Murray, and Gunjan Gupta. “Physiology, vestibular system.” (2018).
Cullen, Kathleen E. “The vestibular system: multimodal integration and encoding of self-motion for motor control.” Trends in neurosciences 35, no. 3 (2012): 185-196.
Cullen, Kathleen, and Soroush Sadeghi. “Vestibular system.” Scholarpedia 3, no. 1 (2008): 3013.
Day, Brian L., and Richard C. Fitzpatrick. “The vestibular system.” Current biology 15, no. 15 (2005): R583-R586.
Fife, Terry D. “Overview of anatomy and physiology of the vestibular system.” Handbook of Clinical Neurophysiology 9 (2010): 5-17.
Khan, Sarah, and Richard Chang. “Anatomy of the vestibular system: a review.” NeuroRehabilitation 32, no. 3 (2013): 437-443.
Smith, Paul F. “The vestibular system and cognition.” Current opinion in neurology 30, no. 1 (2017): 84-89.
Tascioglu, A. Beliz. “Brief review of vestibular system anatomy and its higher order projections.” Neuroanatomy 4, no. 4 (2005): 24-27.
Vidal, Pierre-Paul, K. Cullen, I. S. Curthoys, S. Du Lac, G. Holstein, E. Idoux, A. Lysakowski, K. Peusner, A. Sans, and P. Smith. “The vestibular system.” In The rat nervous system, pp. 805-864. Academic Press, 2015.
Zalewski, Christopher K. “Aging of the human vestibular system.” In Seminars in hearing, vol. 36, no. 03, pp. 175-196. Thieme Medical Publishers, 2015.

When the subject is rocked, the otolith organs detect the changes in the person's head position caused by the rocking motion. This information helps the subject's brain understand that they are in motion.

The signals from the semicircular canals and otolith organs are sent to the brainstem and cerebellum, where they are processed and integrated. This information is then relayed to various parts of the brain, including the thalamus and cortex, which play a role in perception and awareness.

The stimulation of the vestibular system through rocking creates a sensory experience that can be calming and soothing for a baby and adults. This sensory input contributes to the subject's overall sense of body position and movement, helping them to feel secure and relaxed. Additionally, vestibular stimulation can have a sedative effect on the nervous system, promoting a state of calmness and drowsiness, which can be conducive to falling asleep.

The vestibular system is closely linked to other sensory systems, such as vision and proprioception (sensory information from muscles and joints). Together, these sensory inputs help the subject maintain balance and adapt to changes in their environment. Rocking provides a natural way to engage and stimulate the person's vestibular system, making it an effective method for soothing and promoting sleep.

Auditory Stimulation, or sound, can stimulate the vestibular system, but the primary sensory organs responsible for detecting sound are the ears, not the vestibular system itself. The vestibular system, located in the inner ear, primarily senses changes in head position and motion, as well as gravitational forces. It plays a crucial role in balance, spatial orientation, and the perception of motion.

However, sound can indirectly influence the vestibular system in a few ways.

There is a connection between the auditory and vestibular systems in the inner ear. The cochlea, responsible for hearing, and the vestibular system are physically close and share some anatomical structures. Loud or sudden sounds can produce small vibrations within the inner ear, affecting the fluid-filled canals of the vestibular system and potentially causing sensations of dizziness or imbalance in some individuals.

Some research suggests that specific acoustic stimuli, such as low-frequency sounds or infrasound (sound below the range of human hearing), may have subtle effects on the vestibular system and balance.

There are some situations in which auditory stimuli may give rise to sensations of motion or rocking. Certain auditory illusions can create a perceptual experience of motion or spatial displacement. For example, the Shepard-Risset glissando, also known as the “auditory barber pole illusion,” involves a continuously ascending or descending tone that can create the illusion of an endlessly rising or falling pitch, similar to a visual barber pole that appears to rotate infinitely.

Sound can evoke emotional and psychological responses that may indirectly influence one's perception of motion or rocking. For example, calming and rhythmic sounds, such as ocean waves or lullabies, might evoke feelings of relaxation and comfort that are associated with rocking or gentle motion.

The brain often integrates information from multiple sensory modalities, including auditory and vestibular (motion and balance) cues, to create our overall perception of the environment. In some situations, a combination of auditory and other sensory cues might lead to the perception of motion or rocking. For example, being in a room with the sound of waves crashing and feeling gentle physical vibrations could create a sense of motion.

The sensation of rocking or motion induced by visual stimulation is a well-documented phenomenon and can occur in various situations. Here are some ways in which visual stimulation can lead to the perception of rocking or motion.

Op art (optical art) is a genre of visual art that uses geometric patterns, contrasting colors, and other visual techniques to create optical illusions. Some op art patterns, such as those with repetitive lines or shapes, can give viewers the illusion of movement or rocking sensations. For example, the “Bridget Riley” style of op art often produces this effect.

Virtual reality environments and simulators are designed to immerse users in virtual experiences. These systems often incorporate visual cues and motion simulation to create a strong sense of presence. In VR or simulators, the combination of visual motion and physical motion (e.g., in a motion simulator ride) can induce a sensation of rocking or movement.

Watching moving visual patterns, such as waves, swaying trees, or scrolling landscapes, can create the perception of motion or rocking. For example, a video of ocean waves can make viewers feel as though they are gently swaying with the motion of the water.

When exposed to a consistent visual motion for an extended period, the brain may adapt and perceive a stationary reference point as moving in the opposite direction. This phenomenon, known as “motion aftereffect” or “motion adaptation,” can lead to the sensation of rocking or swaying when looking at a stationary object afterward.

Some contemporary art installations are designed to evoke sensory experiences, including the sensation of motion. These installations may use visual effects, lighting, and spatial arrangements to create the perception of movement or rocking.

In some elevators with mirrored walls, the repeated reflections of oneself can create a visual illusion of an endless elevator shaft. This can lead to a perception of descending or ascending motion even when the elevator is stationary.

These sensations are typically the result of perceptual and sensory processes in the brain, rather than actual physical motion. Individual susceptibility to these effects can vary. Some people may be more prone to experiencing rocking sensations through visual stimulation than others.

Rocking is one of the most effective ways to induce a sensation of motion. This can for example be simulated through auditory stimuli by creating sound patterns that mimic the gentle, repetitive movement of rocking. Other ways to create a sensation of motion include simulating the sounds of waves, gentle breezes, or the rhythmic hum of a vehicle in motion. These auditory cues can create a calming effect, promoting relaxation and aiding in the transition to sleep.

The sensation of rocking or other rhythmic movements, also when perceived through auditory stimuli, can help to synchronize brain waves and encourage a state of relaxation and can facilitate sleep. This can make it easier for the subject to fall asleep and maintain sleep throughout the night. By simulating these soothing motions, the device aims to replicate the natural, calming effects of physical rocking, thereby facilitating the sleep process.

Given these challenges, there is a need for an effective, non-invasive device that can help subjects fall asleep more easily and enjoy uninterrupted sleep.

See:
Amemiya, T., Hirota, K., & Ikei, Y., “Tactile flow on seat pan modulates perceived forward velocity,” in 2013 IEEE Symposium on 3D User Interfaces (3DUI), pp. 71-77, 2013.
Anderson, Clare. “Acoustic enhancement of slow wave sleep-timing and tone: “the details create the big picture”.” Sleep 45, no. 10 (2022): zsac191.
Cassandro, Ettore, G. Chiarella, M. Catalano, L. V. Gallo, V. Marcelli, M. Nicastri, and C. Petrolo. “Changes in clinical and instrumental vestibular parameters following acute exposition to auditory stress.” Acta Otorhinolaryngol Ital 23, no. 4 (2003): 251-256.
Cheng, Po-Wen, Chien-Cheng Chen, Shou-Jen Wang, and Yi-Ho Young. “Acoustic, mechanical and galvanic stimulation modes elicit ocular vestibular-evoked myogenic potentials.” Clinical neurophysiology 120, no. 10 (2009): 1841-1844.
Chou, Chen-Han, Wei-Chung Hsu, and Yi-Ho Young. “Ocular vestibular-evoked myogenic potentials via bone-conducted vibration in children.” Clinical Neurophysiology 123, no. 9 (2012): 1880-1885.
Chou, Chen-Han, Wei-Chung Hsu, and Yi-Ho Young. “Ocular vestibular-evoked myogenic potentials via bone-conducted vibration in children.” Clinical Neurophysiology 123, no. 9 (2012): 1880-1885.
Cordero, Leandro, David L. Clark, and Louis Schott. “Effects of vestibular stimulation on sleep states in premature infants.” American journal of perinatology 3, no. 04 (1986): 319-324.
Danilenko, Konstantin V., Evgenii Kobelev, Sergei V. Yarosh, Grigorii R. Khazankin, Ivan V. Brack, Polina V. Miroshnikova, and Lyubomir I. Aftanas. “Effectiveness of visual vs. acoustic closed-loop stimulation on EEG power density during NREM sleep in humans.” Clocks & sleep 2, no. 2 (2020): 172-181.
Diep, Charmaine. “Enhancing Slow Wave Sleep, Cognition and Physiology through Acoustic Stimulation.” PhD diss., Monash University, 2021.
Dumm, Christopher M., Anna C. Hiers, Jeffrey S. Vipperman, George E. Klinzing, and Carey D. Balaban. “Ultrasonic Acoustic Heterodyne Transmission Into the Human Auditory and Vestibular Systems.” In ASME International Mechanical Engineering Congress and Exposition, vol. 84478, p. V001T01A008. American Society of Mechanical Engineers, 2020.
Fiebig, E., K-P. Schaefer, and K-J. SUB. “Form and accuracy of voluntary ocular tracking movements in response to sinusoidally moving acoustic targets.” Journal of Neurology 226 (1981): 77-84.
Halmagyi, G. M., I. S. Curthoys, J. G. Colebatch, and S. T. Aw. “Vestibular responses to sound.” Annals of the New York Academy of Sciences 1039, no. 1 (2005): 54-67.
Harris, C. Stanley, and Henry C. Sommer. “Human equilibrium during acoustic stimulation by discrete frequencies.” The Journal of the Acoustical Society of America 42, no. 5_Supplement (1967): 1188-1188.
Harris, C. Stanley, and Henry C. Sommer. Human equilibrium during acoustic stimulation by discrete frequencies. Vol. 68, no. 7. Aerospace Medical Research Laboratories, Aerospace Medical Division, Air Force Systems Command, United States Air Force, 1968.
Indovina, Iole, and Emiliano Macaluso. “Dissociation of stimulus relevance and saliency factors during shifts of visuospatial attention.” Cerebral Cortex 17, no. 7 (2007): 1701-1711.
Indovina, Iole, Roberta Riccelli, Giuseppe Chiarella, Claudio Petrolo, Antonio Augimeri, Laura Giofre, Francesco Lacquaniti, Jeffrey P. Staab, and Luca Passamonti. “Role of the insula and vestibular system in patients with chronic subjective dizziness: an fMRI study using sound-evoked vestibular stimulation.” Frontiers in behavioral neuroscience 9 (2015): 334.
Indovina, Iole, Roberta Riccelli, Giuseppe Chiarella, Claudio Petrolo, Antonio Augimeri, Laura Giofre, Francesco Lacquaniti, Jeffrey P. Staab, and Luca Passamonti. “Role of the insula and vestibular system in patients with chronic subjective dizziness: an fMRI study using sound-evoked vestibular stimulation.” Frontiers in behavioral neuroscience 9 (2015): 334.
Indovina, Iole, Vincenzo Maffei, Karl Pauwels, Emiliano Macaluso, Guy A. Orban, and Francesco Lacquaniti. “Simulated self-motion in a visual gravity field: sensitivity to vertical and horizontal heading in the human brain.” Neuroimage 71 (2013): 114-124.
Keshavarz, Behrang, Lawrence J. Hettinger, Robert S. Kennedy, and Jennifer L. Campos. “Demonstrating the potential for dynamic auditory stimulation to contribute to motion sickness.” PloS one 9, no. 7 (2014): e101016.
Kossoff, G., and A. E. Khan. “Treatment of vertigo using the ultrasonic generator.” Archives of Otolaryngology 84, no. 2 (1966): 181-188.
Lacquaniti, Francesco, Gianfranco Bosco, Iole Indovina, Barbara La Scaleia, Vincenzo Maffei, Alessandro Moscatelli, and Myrka Zago. “Visual gravitational motion and the vestibular system in humans.” Frontiers in Integrative Neuroscience 7 (2013): 101.
Lim, Rebecca, and Alan M. Brichta. “Vestibular system.” In The mouse nervous system, pp. 661-681. Academic Press, 2012.
Lubner, Rory J., Neil S. Kondamuri, Renata M. Knoll, Bryan K. Ward, Philip D. Littlefield, Derek Rodgers, Kalil G. Abdullah, Aaron K. Remenschneider, and Elliott D. Kozin. “Review of audiovestibular symptoms following exposure to acoustic and electromagnetic energy outside conventional human hearing.” Frontiers in neurology 11 (2020): 234.
Maffei, Vincenzo, Iole Indovina, Emiliano Macaluso, Yuri P. Ivanenko, Guy A. Orban, and Francesco Lacquaniti. “Visual gravity cues in the interpretation of biological movements: neural correlates in humans.” Neuroimage 104 (2015): 221-230.
Manzari, L. “Prolonged bone-conducted vibration in superior semicircular canal dehiscence and in otosclerosis: comparison of the 3D eye movement evaluation.” Acta Otorhinolaryngologica Italica 29, no. 3 (2009): 127.
McCue, M. P., and J. J. Guinan. “Influence of efferent stimulation on acoustically responsive vestibular afferents in the cat.” Journal of Neuroscience 14, no. 10 (1994): 6071-6083.
Palmer, Lyelle L. “Auditory discrimination development through vestibulo-cochlear stimulation.” Academic therapy 16, no. 1 (1980): 53-68.
Parker, Donald E. “The vestibular apparatus.” Scientific American 243, no. 5 (1980): 118-137.
Reschke, Millard F., and D. E. Parner. “Acoustical vestibular stimulation in the guinea pig: Head and eye movements.” The Journal of the Acoustical Society of America 45, no. 1_Supplement (1969): 299-299.
Reybrouck, Mark, Piotr Podlipniak, and David Welch. “Music and noise: Same or different? What our body tells us.” Frontiers in psychology 10 (2019): 1153.
Riccelli, Roberta, Luca Passamonti, Nicola Toschi, Salvatore Nigro, Giuseppe Chiarella, Claudio Petrolo, Francesco Lacquaniti, Jeffrey P. Staab, and Iole Indovina. “Altered insular and occipital responses to simulated vertical self-motion in patients with persistent postural-perceptual dizziness.” Frontiers in Neurology 8 (2017): 529.
Schaefer, K. P., K. J. Suss, and E. Fiebig. “Acoustic-induced eye movements.” Annals of the New York Academy of Sciences 374 (1981): 674-688.
Seno, T., Ogawa, M., Ito, H., & Sunaga, S., “Consistent Air Flow to the Face Facilitates Vection,” Perception, vol. 40, pp. 1237-1240, 2011.
Stewart, Courtney Elaine, Avril Genene Holt, Richard A. Altschuler, Anthony Thomas Cacace, Courtney D. Hall, Owen D. Murnane, W. Michael King, and Faith W. Akin. “Effects of noise exposure on the vestibular system: a systematic review.” Frontiers in neurology 11 (2020): 593919.
Stromberg, Anna-Karin, Ake Olofsson, Magnus Westin, Maoli Duan, and Stefan Stenfelt. “Changes in cochlear function related to acoustic stimulation of cervical vestibular evoked myogenic potential stimulation.” Hearing research 340 (2016): 43-49.
Takigawa, H., H. Sakamoto, and M. Murata. “Effects of infrasound on vestibular function.” Journal of sound and vibration 151, no. 3 (1991): 455-460.
Talamini, Lucia M. “Stabilizing Sleep Through Closed-Loop Acoustic Stimulation; Implications for Obstructive Sleep Apnea Treatment.” In Upper Airway Stimulation in Obstructive Sleep Apnea: Best Practices in Evaluation and Surgical Management, pp. 269-276. Cham: Springer International Publishing, 2022.
Wang, Shou-Jen, Fu-Shan Jaw, and Yi-Ho Young. “Ocular vestibular-evoked myogenic potentials elicited from monaural versus binaural acoustic stimulations.” Clinical Neurophysiology 120, no. 2 (2009): 420-423.
Wit, H. P., and J. D. Bleeker. “Sensitivity of the vestibular system to acoustic stimuli.” Archives of oto-rhino-laryngology 234 (1982): 205-211.
Wit, H. P., J. D. Bleeker, and J. H. Segenhout. “Vestibular and cochlear responses to acoustic transients: Some properties of whole-nerve action potentials in pigeons.” Acta Oto-Laryngologica 92, no. 1-6 (1981): 409-422.

U.S. Patents and Published Patent Application Nos. U.S. Pat. Nos. 6,014,117; 6,065,057; 6,118,449; 6,309,306; 7,128,705; 7,279,073; 7,717,841; 7,815,313; 7,893,935; 7,940,748; 7,952,483; 7,975,241; 8,092,398; 8,159,354; 8,235,918; 8,242,940; 8,331,561; 8,365,244; 8,427,325; 8,427,360; 8,428,255; 8,529,477; 8,594,381; 8,692,845; 8,700,469; 8,713,634; 8,740,820; 8,831,278; 9,041,587; 9,080,868; 9,167,998; 9,205,929; 9,298,985; 9,352,157; 9,370,302; 9,427,659; 9,558,676; 9,690,371; 9,731,125; 9,788,714; 9,795,335; 9,828,542; 9,994,228; RE25285; U.S. Pat. Nos. 10,004,940; 10,035,026; 10,053,226; 10,096,213; 10,099,030; 10,133,344; 10,231,614; 10,258,259; 10,397,539; 10,569,084; 10,602,927; 10,675,465; 10,696,387; 10,716,469; 10,814,095; 10,958,950; 11,033,453; 11,092,979; 11,115,769; 11,259,134; 11,266,919; 11,273,344; 11,337,606; 11,337,882; 11,382,383; 11,389,059; 11,490,809; 11,504,051; 11,526,808; 11,571,586; 11,601,772; 11,612,316; 11,712,162; 11,879,735; 11,911,617; 11,969,666; 12,003,944; 12,013,978; 20040062879; 20060022808; 20070038268; 20080120425; 20080120697; 20080120705; 20080140137; 20090002142; 20100142448; 20110148672; 20110150215; 20110150216; 20120105483; 20120121138; 20120134543; 20120150294; 20120182206; 20120218285; 20130030330; 20130097677; 20140125511; 20140152792; 20150022380; 20150057719; 20150062000; 20150068052; 20150080753; 20150196780; 20150273179; 20150290490; 20150360773; 20160007849; 20160045733; 20160167672; 20160262608; 20160272340; 20170010671; 20170262047; 20170293356; 20170295353; 20170304616; 20170304618; 20170344706; 20180008141; 20180221621; 20190114841; 20190167095; 20190200862; 20190204909; 20190232113; 20190346865; 20200060920; 20200138364; 20200166993; 20200169823; 20200169827; 20200197825; 20200214559; 20200253320; 20200305708; 20200306535; 20200324073; 20200397288; 20200401938; 20210125732; 20220011110; 20220133212; 20220146841; 20220244726; 20230019068; 20230125209; 20230209290; 20230210442; 20240130661; and 20240163412.

US 2013/0096368 A1 (Devroey) describes a “Uterine Sound and Motion Simulation Device” that uses bladders to create a sense of motion and speakers to produce sounds mimicking the womb.

U.S. Pat. No. 7,346,949 B2 (Kamrin-Balfour) is for an “Infant soothing and sleep aid” discloses a device with a vibration mechanism and a white noise generator.

U.S. Pat. No. 9,764,110 B2 (Larson) is for a “Binaural sleep inducing system,” describes a system that monitors brain activity and adjusts binaural beats to guide the user into a sleep state.

U.S. Pat. No. 11,528,547 B2 (Redfield) describes a pad that provides vestibular and somatosensory stimulation through bone conduction of sound waves.

Dreampad (Integrated Listening Australia, dreampadsleep.com) is a product that uses “Intrasound Technology” that delivers music through vibrations that are transmitted through bone conduction. It is marketed as a way to relieve stress and improve sleep.

Zen Vibez (Indiegogo Campaign, www.indiegogo.com/projects/zen-vibez-neural-stimulation-wearable-device#/): This is a “Neural Stimulation Wearable Device” that uses vibrations to create a brainwave entrainment effect. The device is designed to be user-friendly and can also incorporate binaural beats.

### SUMMARY OF THE INVENTION

The present technology provides a system and method for inducing a sensation of motion in a person to help them fall asleep. It uses sound and visual stimulation, and/or electromechanical transducers that create vibrations.

A swaying or rocking sensation can also be induced by visual stimulation. This phenomenon is known as vection, or more specifically, visually induced motion sickness (VIMS) or visual vertigo.1 (see Ugur E. Investigation of the Correlation Between the Visually Induced Motion Sickness Susceptibility Questionnaire and the Turkish Motion Sickness Susceptibility Questionnaire. J Audiol Otol. 2024 January; 28(1):36-43. doi: 10.7874/jao.2023.00122. Epub 2023 Oct. 20. PMID: 37857370; PMCID: PMC10808388.)

Our brain constantly integrates information from various sensory systems to determine our body's position and motion in space. These systems include: visual system (what our eyes see); vestibular system (located in the inner ear, it senses head movements and gravity); proprioceptive system (sensors in our muscles and joints that tell us about our body's position. However, in many situations, the visual system can be dominant. If a large portion of your visual field is moving, the brain might prioritize that visual input over the lack of corresponding signals from your vestibular and proprioceptive systems, leading to an illusion of self-motion.

Virtual reality (VR) experiences are a prime example where moving visual scenes can induce a strong sensation of self-motion, often leading to “cybersickness” with symptoms like dizziness, nausea, and disorientation. Another example is Optokinetic Stimuli. Patterns of moving stripes or dots (optokinetic stimuli) can induce a feeling of self-motion (vection) and even involuntary eye movements (optokinetic nystagmus). If these visual patterns simulate a rocking or swaying motion, the brain can interpret that as actual physical movement.

To specifically induce a rocking or swaying sensation visually, one can present a large, rhythmic, oscillating visual field. For instance, a display showing a scene gently moving from side to side, or a pattern that appears to swing, can trick the brain into perceiving that the observer is rocking. An image of the clouds in the sky moving from side to side as perceived by a person looking at the sky while lying on the boat rocking gently back and forth on a lake is particularly effective. This creates a sensory conflict where the visual input suggests motion, while the inner ear and body sensors indicate stillness. The brain attempts to resolve this conflict, often by generating the illusion of self-motion.

While this visual induction of motion is well-documented, especially in the context of motion sickness, it can also be used for therapeutic purposes, including potentially influencing sleep, similar to how auditory stimulation can.

The vibrations can also induce the sensation of swaying or rocking. These vibrations are designed to mimic a feeling of gentle, cyclic rotation, which is known to be soothing and can help induce sleep. The system also uses auditory stimuli, specifically binaural beats, which are created by playing two slightly different frequencies in each ear. This combination of vibrational and auditory stimulation is intended to create a powerful sleep-inducing experience. The cyclic rotation is ideally in the range of 0.2 to 1 Hz, a frequency particularly effective for inducing sleep.

The present invention addresses these needs by providing a device and method for inducing a sensation of motion in a subject through the use of auditory stimuli.

The device comprises first and second acoustic transducers, a control unit operatively connected to the first and second transducers, wherein the control unit is adapted to deliver the stimuli to the first and second transducers and a measuring device adapted to detect a sleep state of the subject, wherein the measuring device is operatively connected to the control unit.

The device is preferably capable of providing personalized auditory stimuli that can be adjusted based on real-time feedback from the subject's physiological state. By doing so, it can cater to the specific needs of each individual, promoting relaxation and facilitating the natural sleep process or influence a sleep state.

The term “sleep state” should be understood to also encompass the wake state, wakefulness and/or vigilance of the subject. This allows the device to monitor and respond to different levels of alertness and relaxation, ensuring that the auditory stimuli can be adjusted to effectively promote sleep regardless of the subject's initial state.

The present invention encompasses the concept of inducing a sensation of motion through auditory or sound stimuli. A sensation of motion can be defined as the subjective experience of feeling movement without actual physical displacement. This sensation can be achieved through various auditory cues that simulate or stimulate the perception of motion, such as rocking, swaying, or rhythmic movements.

The auditory or sound stimuli delivered by the device are designed to induce a sensation or perception of motion, which can be related to the concept of neuromodulation. Neuromodulation refers to the process of altering nerve activity through targeted delivery of a stimulus, such as electrical impulses, chemical agents, or in this case, auditory stimuli. By influencing neural pathways, neuromodulation aims to regulate or normalize nervous system functions.

In The auditory or sound stimuli simulate or stimulate the sensation or perception of motion, such as rocking or swaying, which can affect the brain's sensory processing and neural activity. This gentle modulation of neural pathways helps to synchronize brain waves and promote a state of relaxation, facilitating the transition to sleep. The ability of the auditory stimuli to induce a sensation of motion is an aspect of the device's effectiveness in improving sleep quality and helping the subject to achieve and maintain restful sleep states. The auditory stimuli preferably operate through a through neuromodulation mechanism.

The device utilizes these principles by generating auditory stimuli that can induce a sensation of motion. The control unit is designed to deliver these stimuli through the first and second headphones, which can vary in type and configuration.

The technology provides two discrete modalities. Vection is a feeling of movement when the subject is actually immobile. It's also known as an illusion of self-motion or spatial disorientation. Vection can be induced by visual stimulation of actual motion. Vection may also be induced by movement of a virtual object within a soundscape. Further, vection may be induced by stimulation of the vestibular system by false signals, such as vibration (e.g., in the acoustic or infrasonic range). Motion sickness may be associated with inconsistent or unsynchronized sensory signals from the various sensory modalities.

See, U.S. Patent and Pub. Pat. Application Nos. 20240203284; 20240163412; 20240013501; 20240003872; 20230414697; 20230210442; 20230125209; U.S. Pat. No. 11,571,586; 20220354901; 20220244726; 20220238083; 20220221156; 20220133212; 20210125732; 20210038927; 20200401938; 20200305708; D895002; 20200214559; 20200197825; U.S. Pat. No. 10,684,469; 20200169827; D885435; D884738; 20200060920; 20200062466; D875824; D873859; 20190232113; 20190200862; 20190167095; 20190114841; U.S. Pat. No. 10,258,259; 20190104330; D835673; 20180312303; 20180276123; 20180270463; 20180221621; 20180008141; U.S. Pat. Nos. 9,828,542; 9,795,335; 20170300196; 20170293356; 20170285344; 20170282947; 20170233148; 20170220110; 20170210293; 20170213092; U.S. Pat. No. 9,681,835; 20170010671; 20160313635; 20160272340; 20160262608; 20160167672; 20160103326; 20160033770; 20160007849; 20150360773; 20150290490; 20150273179; 20150196780; 20150180944; 20150168728; 20150168729; 20150080753; 20150068052; 20150062000; 20150062165; 20140285403; 20140152792; U.S. Pat. No. 8,700,469; 20130292328; 20120218285; 20120182206; 20120134543; 20120118722; 20120121138; 20120105483; 20110279669; 20110280976; 20110148672; 20110150215; 20110150216; 20110043537; 20100142448; U.S. Pat. No. 7,734,106; 20100135399; U.S. Pat. No. 7,667,700; 20090002142; 20080120425; 20080120697; 20080120705; 20080064513; 20070195879; U.S. Pat. No. 7,192,614; 20060022808; U.S. Pat. No. 6,723,837; 20040062879; 20030211470; 20030132860; U.S. Pat. No. 6,581,412; 20030073922; U.S. Pat. Nos. 6,364,845; 6,309,306; 6,187,748; 6,080,566; 6,065,057; 5,928,927; 5,841,439; 5,829,446; 5,676,870; 5,662,908; 5,557,341; 5,338,842; and 5,239,066.

Audio and visual signals may be presented to a subject in traditional manner, such as TV, projector, speakers, spatial audio, headphones, bone conduction headphones, VR googles, etc.

See, U.S. Patent and Pub. Pat. Application Nos. 20240233268; U.S. Pat. No. 12,034,954; 20240135647; 20240077957; 20240057926; 20240061510; 20240062467; 20230393650; 20230381515; 20230372662; 20230360227; 20230260179; 20230231984; 20230210442; 20230186569; 20230159046; 20230145623; 20230123809; 20230116190; 20230106673; U.S. Pat. No. 11,615,740; 20230062244; 20230068679; 20230054759; 20230048398; 20230030201; 20230035360; U.S. Pat. No. 11,562,550; 20220413609; 20220394181; 20220381898; 20220366597; 20220357804; 20220291750; 20220217389; U.S. Pat. No. 11,381,743; 20220133212; U.S. Pat. Nos. 11,321,857; 11,308,920; 20220087813; 20220086586; 20210398314; 20210383115; 20210354286; 20210281638; 20210231796; 20210225049; 20210224319; 20210223549; U.S. Pat. Nos. 11,064,387; 11,057,614; U.S. Pat. No. 11,048,082; 20210125664; 20210118089; 20210065427; 20210065428; 20210064124; 20210033729; 20210004075; U.S. Pat. No. 10,864,422; 20200364901; 20200357299; 20200356172; U.S. Pat. No. 10,783,800; 20200271450; 20200242790; 20200132832; 20200107075; 20200097088; U.S. Pat. No. 10,596,371; 20200089956; 20200092539; U.S. Pat. No. 10,589,679; 20200084367; 20190394484; 20190387168; 20190349213; 20190328275; 20190328458; 20190302883; 20190286234; 20190273911; 20190246094; 20190243371; 20190189086; 20190178654; 20190180669; 20190159715; U.S. Pat. No. 10,300,603; 20190083739; 20190080442; 20190050998; 20190029513; 20180365804; 20180359414; 20180332421; 20180299258; 20180286105; 20180247580; 20180239430; U.S. Pat. No. 9,996,149; 20180121069; 20180104578; 20180109900; 20180041709; 20180000407; 20180000179; 20170366866; 20170366867; 20170345212; U.S. Pat. No. 9,807,384; 20170280199; 20170280090; 20170279957; 20170243324; 20170243319; U.S. Pat. Nos. 9,734,524; 9,661,307; 20170102772; U.S. Pat. No. 9,606,450; 20170039926; 20160349837; 20160314564; 20160235323; 20160225188; 20160216130; 20160167672; 20160095513; 20160088289; 20160035139; 20160035132; 20160035122; 20150325027; 20150326932; U.S. Pat. No. 9,037,527; 20150022380; 20140375679; 20140160341; 20140152792; 20140119581; 20140072213; U.S. Pat. No. 8,401,081; 20120182206; 20120134543; 20120121138; 20120105483; 20120050140; 20120050141; 20120050044; 20120050142; 20120050143; 20120045091; 20110282130; 20110228753; 20110135098; 20110043537; U.S. Pat. No. 7,856,055; 20100106010; 20090046140; 20080278682; 20080108883; 20080030429; 20070254270; 20070239001; 20070126938; 20070126932; 20070126864; 20070028286; 20070019866; 20070022217; 20060059024; U.S. Pat. No. 6,944,315; 20050069839; U.S. Pat. Nos. 6,734,884; 6,631,240; 6,515,688; 6,491,525; 20020106617; U.S. Pat. Nos. 6,428,449; 6,341,372; 6,271,842; 6,233,545; 6,226,001; 6,222,554; 6,104,406; 6,094,196; 6,081,271; 6,069,632; 6,064,389; 6,014,145; 5,923,324; 5,903,271; 5,900,879; 5,883,628; 5,829,446; 5,767,855; 5,555,354; 5,528,735; 5,517,021; 5,454,043; 5,360,971; 4,549,184.

Stimulation of the vestibular organs (other than the natural inertial inputs) is known, but not generally deployed. This is due, in part, to the generally high amplitude mechanical signals required to achieve robust stimulation. However, infrasonic stimulation can cause vestibular system activation. However, the required physical displacements to achieve power transfer at low frequencies can limit the feasibility.

Therefore, one aspect of the technology provides a controlled vibrational stimulation conducted through the skull (such as through bone conduction headphones) to stimulate the utricle and saccule, and/or the semicircular canals. The vibrational stimulation may extend from 1-500 Hz, for example, and especially where a separate acoustic source is available, the range may be 1-35 Hz, for example.

The lower frequencies may be stimulated by a rotating or reciprocating mass (e.g., pager motor). In order to simulate motion or stimulate the vestibular system to perceive motion, the right at left sides may be stimulated with different signals, e.g., different fundamental frequencies. By keeping the stimulation within the infrasonic range (<˜20 Hz), auditory stimulation is minimized (except for harmonics).

While operating through distinct neural pathways, an effect similar to binaural beats may be used wherein the right and left stimulus signals differ by a small amount, e.g., 0.2-2 Hz.

An effect corresponding to isochronic tones may be achieved by having dial vibrational emitters operating at similar but not identical frequencies, operating neat each ear. One advantage to using multiple transducers is that the available signal amplitude at the target organ is doubled for the same maximum stimulus amplitude. The stimuli for each ear may differ.

See, U.S. Patent and Pub. Pat. Application Nos. U.S. Pat. Nos. 12,029,573; 12,029,701; 20240197558; 20240172949; 20240148592; 20240148324; 20240143136; 20240108526; 20240100294; 20240033465; 20240029694; 20240009069; 20240001068; 20240004218; 20230406203; 20230404466; 20230398324; 20230390513; 20230389714; 20230372662; 20230377543; 20230379638; 20230355917; 20230347103; 20230338691; 20230329631; 20230320645; 20230321458; 20230321392; 20230320691; 20230310254; 20230310792; 20230284726; 20230277070; 20230277123; 20230277406; 20230256192; 20230256191; 20230210713; 20230201069; 20230201550; 20230191076; 20230190189; 20230165753; 20230166072; 20230166122; 20230165745; 20230158270; U.S. Pat. No. 11,654,082; 20230142810; U.S. Pat. No. 11,642,039; 20230128812; 20230113072; 20230111776; 20230106753; 20230104621; 20230084900; 20230077519; 20230073174; 20230066359; 20230069700; 20230066293; 20230062659; 20230066923; 20230055346; 20230049966; 20230030406; 20230022546; 20230001127; 20220409849; 20220408205; 20220408147; 20220401749; 20220387749; 20220347474; 20220339062; 20220343882; 20220331606; 20220273909; 20220277852; 20220273907; 20220265959; 20220262332; 20220257454; 20220257453; 20220249803; 20220233860; 20220233244; 20220233119; 20220217476; 20220212030; 20220210586; 20220176151; 20220160995; 20220108784; 20220088041; 20220095067; 20220088343; 20220088408; 20220086560; 20220072267; 20220047448; 20220008746; 20220001190; 20210407646; 20210393473; 20210394069; 20210386963; 20210386964; 20210366591; 20210353957; 20210353904; 20210345947; 20210338970; 20210338973; 20210345059; 20210337312; 20210308413; 20210307684; 20210304788; 20210290480; 20210296003; U.S. Pat. No. 11,122,356; 20210260404; 20210244605; 20210213237; 20210218380; 20210173613; 20210128058; 20210113431; U.S. Pat. No. 10,952,011; 20210069488; U.S. Pat. No. 10,943,407; 20210046276; 20210041953; 20210030997; 20210030998; 20210021947; 20210008335; U.S. Pat. No. 10,885,800; 20200393902; U.S. Pat. No. 10,864,347; 20200375842; 20200382884; 20200368487; 20200368491; 20200330322; 20200316367; 20200305739; U.S. Pat. No. 10,786,666; 20200289694; 20200286456; 20200269123; 20200245931; U.S. Pat. No. 10,688,274; 20200178875; 20200101260; 20200097249; 20200086078; 20200077939; 20200082927; 20200069966; 20200038658; 20200029881; 20200030596; 20200030192; 20200008739; 20200000348; 20190387998; 20190374742; 20190328996; 20190325767; 20190321585; 20190321583; 20190282779; 20190247662; 20190223747; 20190201658; 20190201691; 20190189259; U.S. Pat. No. 10,314,407; 20190167174; 20190164530; 20190143073; 20190117106; 20190117933; 20190117105; U.S. Pat. No. 10,264,357; 20190105461; 20190082990; U.S. Pat. No. 10,232,139; 20190070386; 20190060605; 20190030277; 20190008450; 20180368717; 20180344968; 20180333574; 20180317795; 20180318544; 20180321898; 20180261332; 20180250494; 20180236232; 20180193512; 20180133507; 20180110960; U.S. Pat. No. 9,886,965; 20180035236; 20180021624; 20170368329; 20170368297; 20170365101; 20170361059; 20170340855; 20170311832; 20170312476; 20170286536; 20170266477; 20170224990; 20170228512; 20170193169; 20170182285; 20170173296; 20170143935; 20170149945; 20170119994; U.S. Pat. No. 9,629,976; 20170061760; 20170053637; U.S. Pat. No. 9,576,501; 20170039045; 20170032098; 20160346545; 20160302718; 20160287738; U.S. Pat. No. 9,457,166; 20160270656; 20160262974; 20160205489; U.S. Pat. No. 9,272,118; 20160055842; 20160030702; 20160029946; 20160022167; 20160022206; 20160015289; 20160008568; 20160005320; 20150359432; 20150342493; 20150313949; 20150297109; 20150296288; 20150283019; 20150174362; 20150092972; 20150071448; 20150038776; 20150038869; U.S. Pat. No. 8,932,218; 20140350706; 20140343354; 20140336473; 20140272839; 20140275741; 20140236759; U.S. Pat. No. 8,801,591; 20140167968; 20140164455; 20140154647; 20140114207; 20140094666; 20140052033; 20130236865; 20130234823; 20130216055; 20130202119; 20130203019; 20130184552; 20130177883; 20130172663; 20130131537; 20130089845; 20130010967; 20120225411; 20120150545; 20120149973; 20120130215; 20120112909; 20120029379; 20110319482; 20110313315; 20110298706; 20110251535; 20110184247; 20110172726; 20110122130; 20110105938; 20110010014; 20100286497; 20100280124; 20100239096; 20100240458; 20100168504; 20100152254; 20100076253; 20100028841; 20100010289; 20090318826; 20090209834; 20090149361; 20090118569; 20090005837; 20080304691; 20080269629; 20080269652; 20080244003; 20080125620; 20080033252; 20070287892; 20070282216; 20060281543; 20060252979; 20060250392; 20060249004; 20060136009; 20060116600; 20060030907; 20050234290; 20050182287; 20040175438; 20040002635; 20030225095; 20020151527; 20020146469; U.S. Pat. Nos. 6,146,333; 6,081,743; RE36348; U.S. Pat. Nos. 5,666,433; 5,518,497; 5,365,939; 5,356,368; 5,213,562; 5,135,468; 5,036,858; 4,775,805; 4,575,688; 4,122,910; 3,939,751; 3,735,014; and 3,647,928.

Typically, where multimodal stimulation is employed, all modalities present the same vection goal.

The system and method is not limited to sleep induction, and therefore may find application in virtual/augmented/modified reality systems, motion sickness treatments, etc. However, a preferred embodiment of the technology is controlled to induce drowsiness in a human through a vection pattern of rocking or rolling, or complex motion patterns. Concurrently with the vection stimuli directed to the vestibular organs, audio and/or visual stimuli consistent with the motion may be presented. Further, the audio and/or visual information may also include other known cognitive stimuli which promote drowsiness or sleep. The vection stimuli and/or cognitive stimuli may be adaptive to biometric indicia, such as inferred sleep stage.

The control unit plays an important role in delivering the auditory stimuli necessary to induce a sensation of motion. This control unit can be designed in various ways to enhance its functionality and user experience.

One configuration of the control unit involves storing the auditory stimuli within its memory. These pre-recorded stimuli can be played back as needed, ensuring consistent delivery of the desired sounds to the headphones. Alternatively, the control unit can be equipped with the capability to generate the stimuli in real-time, allowing for more dynamic and responsive sound patterns that can be adapted to the subject's needs.

The control unit can offer a variety of auditory stimuli that the subject can select from. These stimuli can include different types of sounds designed to induce a sensation of motion, such as rocking, swaying, or other rhythmic movements. The selection of stimuli can be made manually by the subject and/or automatically based on various factors such as the detected sleep state of the subject, ambient noises, the time of day, or the brightness of the environment.

The control unit may be a smartphone, Raspberry Pi 4 or 5, Arduino, or other real-time control processor.

See, U.S. Patent and Pub. Pat. Application Nos. 20240230628; 20240225465; U.S. Pat. No. 12,029,532; 20240206841; 20240210221; 20240188876; 20240180486; 20240180436; 20240156355; 20240156364; 20240138776; 20240127384; 20240101997; 20240081657; 20240074709; 20240078509; 20240068839; 20240043641; 20240041386; 20240041251; U.S. Pat. Nos. 11,892,746; 11,885,887; 20240023838; 20240001068; 20230413432; 20230398353; 20230388576; U.S. Pat. No. 11,826,129; 20230337961; 20230329622; 20230323433; 20230320817; 20230293754; 20230293026; 20230293810; U.S. Pat. No. 11,763,949; 20230277068; 20230277071; 20230217195; 20230190160; 20230191022; 20230174949; 20230140419; 20230124940; 20230106450; 20230075084; 20230043376; 20220361802; U.S. Pat. Nos. 11,495,111; 11,478,606; 20220322999; 20220322938; 20220294796; 20220280370; 20220273269; 20220241530; 20220241540; 20220219001; 20220192549; 20220183618; 20220176057; 20220160309; 20220160991; U.S. Pat. Nos. 11,324,869; 11,320,588; 20220117558; 20220071511; 20220068154; U.S. Pat. No. 11,250,529; 20220023561; 20220022809; 20220003577; 20220003676; 20210409286; 20210393176; 20210370070; 20210366592; 20210346699; 20210338092; 20210339023; 20210308400; 20210275087; U.S. Pat. Nos. 11,096,660; 11,097,103; 20210228869; 20210212889; 20210183488; 20210145359; 20210137398; 20210137402; 20210128061; 20210128000; 20210113801; 20210073983; 20210055179; 20210022641; 20210008281; 20210000383; 20210001157; U.S. Pat. No. 10,877,444; 20200392567; 20200390365; 20200375471; 20200367761; 20200329978; 20200333829; U.S. Pat. No. 10,810,907; 20200323461; 20200305736; 20200297982; 20200268301; 20200222276; 20200205736; 20200196903; 20200170544; U.S. Pat. No. 10,629,308; 20200118456; 20200113442; 20200113511; 20200107734; 20200075160; 20200069193; 20200064337; 20200064172; 20200054520; 20200029836; 20200029840; 20190374695; 20190345488; 20190346417; 20190336038; 20190313944; 20190298193; 20190290144; 20190283026; 20190247664; 20190250715; U.S. Pat. No. 10,349,148; 20190183255; 20190175397; 20190168248; 20190143073; 20190117863; 20190076024; 20190050622; 20190038227; 20180357886; 20180333306; 20180317783; 20180310847; 20180303386; 20180256029; 20180214030; 20180161502; 20180147375; U.S. Pat. No. 9,968,296; 20180091961; 20180070831; U.S. Pat. No. 9,909,942; 20180049652; 20180052958; 20180027077; U.S. Pat. No. 9,870,457; 20170368348; 20170367578; 20170316487; 20170305014; 20170293003; 20170279612; 20170259072; 20170209083; U.S. Pat. No. 9,690,368; 20170139233; 20170039822; 20170020447; 20170000359; 20160374567; 20160359992; 20160346449; 20160342753; 20160331273; 20160324432; 20160310149; 20160314279; 20160256097; 20160243701; 20160210747; 20160203663; 20160151553; 20160133151; 20160106910; 20160106911; 20160070702; 20160051195; 20150335691; 20150272464; 20150269383; 20150251074; 20150142082; 20140336452; 20140324118; 20140206412; 20140088442; 20140073486; 20140057232; 20130274642; 20130190556; U.S. Pat. No. 8,288,678; 20120251989; 20120188084; and 20080118523.

In addition to delivering pre-recorded stimuli, the control unit can also generate new stimuli based on real-time feedback and environmental cues. This allows for a highly personalized and adaptive approach to sleep induction, ensuring that the auditory stimuli are always tailored to the current needs of the subject.

The control unit can be a mobile device, such as a cell phone or a dedicated sleep aid device. This mobile configuration allows for greater flexibility and portability, enabling the subject to use the device in various settings without being tethered to a fixed location. The control unit can be equipped with a battery for power, ensuring it can operate independently for extended periods.

Additional features of the control unit may include a display for user interface and feedback, volume controls to adjust the intensity of the auditory stimuli, and/or a charger for recharging the battery. Other features can include connectivity options such as Bluetooth or Wi-Fi for wireless communication with the headphones, a user interface for selecting and customizing stimuli, and potentially an app for additional control and monitoring functions.

Additional features might include a sleep tracking function to monitor and analyze the subject's sleep patterns, alarms or wake-up features, and/or integration with other smart home devices to create a comprehensive sleep-enhancing environment.

The control unit is preferably designed to adapt the auditory stimuli delivered to the subject based on the detected sleep state. The control unit receives preferably real-time data, from the measuring device.

When it is referred to the control unit delivering the stimulus, it preferably means that the control unit is responsible for generating and transmitting the auditory stimuli to the headphones based on the data received from the measuring device. The control unit is preferably designed to adjust the stimulus independently according to the detected sleep state. In its simplest form, this could mean turning off the stimulus and/or the device when it is detected that the subject has fallen asleep and reached any sleep state.

However, it is preferable for the control unit to modify the stimulus based on the specific detected sleep state. For example, if the control unit detects that the subject has entered a light sleep stage, it can adjust the auditory stimuli to promote a transition into a deeper sleep stage. Conversely, if the subject is in a deep sleep stage, the control unit might reduce the stimuli to maintain that state without causing disruptions. The control unit is preferably designed to deliver stimuli that are modulable and/or selectable based on input from the measuring device.

This capability to modify the stimulus preferably allows the system to help the subject achieve certain sleep states more quickly or shorten the duration of less desirable sleep states. The control unit might enhance the stimulus to promote deep sleep or REM sleep once light sleep is detected or reduce the stimulus to maintain deep sleep longer. The goal of such a configuration is to help the subject enter a desired sleep state more quickly and/or remain in that state for a longer period, thus enhancing the overall quality of sleep.

For example, in cases where the measuring device detects that the subject is in REM sleep, the control unit can deliver stimuli that support the maintenance of this stage, which is crucial for cognitive functions and memory consolidation. If the subject is detected to be in a wakeful state or experiencing disturbed sleep, the control unit can adjust the stimuli to facilitate a return to a more restful state.

This embodiment allows for a highly personalized and adaptive sleep induction process. By continuously monitoring the subject's physiological parameters, the device can make precise adjustments to the auditory stimuli to optimize the sleep environment for the subject. This ensures that the stimuli remain effective and appropriate throughout the entire sleep cycle.

See, U.S. Patent and Pub. Pat. Application Nos. 20240226476; 20240225532; 20240225466; 20240226497; 20240227260; 20240232746; 20240226477; 20240225890; 20240226563; 20240226482; 20240225522; 20240225523; U.S. Pat. Nos. 12,029,852; 12,029,573; 12,029,324; 12,029,583; 12,029,713; 12,033,758; 12,033,485; 20240215908; 20240216339; 20240219237; 20240216163; 20240216692; 20240207265; 20240208981; 20240206822; 20240206805; 20240211566; 20240211719; 20240207615; 20240207554; 20240207566; 20240206810; 20240206804; 20240197704; 20240203586; 20240203558; 20240203585; 20240203602; 20240197199; 20240197249; 20240189328; 20240188883; 20240181185; 20240180498; 20240183936; 20240173499; 20240179550; 20240173516; 20240173559; 20240165410; 20240165422; 20240165411; 20240169208; 20240164724; 20240165370; U.S. Pat. No. 11,986,451; 20240145085; 20240138758; 20240138759; 20240139446; 20240139448; 20240131287; 20240131338; 20240130680; U.S. Pat. No. 11,963,792; 20240129166; 20240129779; 20240129778; 20240125915; 20240122535; 20240122550; 20240122544; 20240122548; 20240118748; 20240115550; 20240115193; 20240115144; 20240120055; 20240108665; 20240108602; 20240109553; 20240108148; 20240108242; 20240108230; 20240108843; 20240091476; 20240090829; 20240096463; 20240094821; 20240081729; 20240081730; 20240081732; U.S. Pat. Nos. 11,925,441; 11,925,447; 20240075225; 20240074709; 20240079763; 20240081012; 20240080998; 20240077526; 20240078940; 20240066249; 20240065618; 20240065615; 20240071597; 20240071624; 20240065619; U.S. Pat. Nos. 11,911,351; 11,911,181; 11,914,842; 20240062872; 20240058278; 20240049974; 20240050451; 20240049811; 20240050762; 20240050028; 20240041397; 20240042149; 20240042164; 20240041340; 20240041396; 20240033457; 20240033459; 20240032859; 20240032860; 20240038343; 20240032836; U.S. Pat. No. 11,883,188; 20240024597; 20240024617; 20240024619; 20240016447; 20240022565; 20240021313; 20240012887; 20240009420; 20240013643; 20240008751; 20240008809; 20240000344; 20240008205; 20240000328; 20240000204; 20240000205; 20240000206; 20230417544; 20230420124; 20230414940; 20230404475; 20230405250; 20230404793; 20230404476; 20230404466; 20230405263; 20230397880; 20230397851; 20230397852; 20230390228; 20230390513; 20230389862; 20230389861; 20230389870; 20230380758; 20230380692; 20230380776; 20230377114; 20230376114; 20230371890; 20230367000; 20230364368; 20230363699; 20230363700; 20230368901; 20230364377; 20230364427; 20230355169; 20230346305; 20230347147; 20230346237; 20230343435; 20230339518; 20230337937; 20230338691; 20230337924; 20230337961; 20230341944; 20230338677; U.S. Pat. No. 11,793,425; 20230335287; 20230329583; 20230320656; 20230320893; U.S. Pat. No. 11,779,557; 20230310781; 20230310364; 20230317269; U.S. Pat. No. 11,771,367; 20230306267; 20230301599; U.S. Pat. No. 11,770,197; 20230293084; 20230293049; 20230293096; 20230298761; 20230284969; 20230284726; 20230277121; 20230282363; 20230277135; 20230277056; 20230277074; 20230270350; 20230270378; 20230264030; 20230263465; 20230248927; 20230248320; 20230248934; 20230240595; 20230245780; 20230241337; U.S. Pat. No. 11,712,557; 20230233794; 20230233793; 20230233858; 20230238112; 20230238113; 20230239698; 20230226308; 20230228443; 20230225676; 20230218845; 20230218186; 20230218844; 20230218225; 20230214470; 20230215557; 20230210468; 20230210395; 20230210385; 20230206486; 20230200677; 20230200751; 20230190140; 20230191063; 20230191053; 20230190121; 20230190197; 20230190201; 20230190206; 20230197265; 20230181102; 20230181100; 20230181073; 20230173221; 20230172486; 20230172514; 20230172874; 20230173207; U.S. Pat. No. 11,670,410; 20230165510; 20230165498; 20230166072; 20230170097; 20230165508; 20230165527; U.S. Pat. No. 11,660,005; 20230157637; 20230158273; 20230148954; 20230148956; 20230148952; 20230149330; 20230149711; 20230144677; 20230144761; 20230134935; 20230138935; 20230136992; 20230129375; 20230128912; 20230128706; 20230120071; 20230123806; 20230111477; 20230111776; 20230114269; 20230114650; 20230114833; 20230113714; 20230115575; 20230112187; 20230112231; 20230107369; 20230106450; 20230104621; 20230107454; 20230107624; 20230105636; 20230106024; 20230108715; 20230095780; 20230099622; 20230097263; 20230096307; 20230086651; 20230085305; 20230084205; 20230081794; U.S. Pat. Nos. 11,602,513; 11,602,512; 20230073907; 20230061348; 20230065695; 20230062794; 20230061823; 20230061149; 20230059947; U.S. Pat. No. 11,583,510; 20230048000; 20230046169; 20230049642; 20230049849; 20230050908; 20230037360; 20230039091; 20230036312; 20230031563; 20230035257; 20230033963; 20230033976; 20230023391; 20230012989; 20230000396; 20230000428; 20230000829; 20230000429; 20230000380; 20230000381; 20220409082; 20220409187; U.S. Pat. No. 11,534,608; 20220404374; 20220406420; 20220401673; 20220401019; 20220401689; 20220401378; 20220406450; 20220406453; 20220401739; 20220400952; 20220395681; 20220395181; 20220387368; 20220386946; 20220387366; 20220386947; 20220386945; 20220386944; 20220387749; 20220378367; 20220385458; 20220382844; 20220370752; 20220370802; 20220375590; 20220375591; 20220369936; U.S. Pat. No. 11,504,347; 20220362221; 20220366466; 20220354431; 20220354385; 20220347412; 20220346565; 20220348116; 20220347426; U.S. Pat. No. 11,484,268; 20220339131; 20220339380; 20220338755; 20220344057; 20220339398; U.S. Pat. Nos. 11,478,638; 11,483,619; 11,478,184; 20220322993; 20220322999; 20220323000; 20220322998; 20220322852; 20220312982; 20220319690; 20220313945; 20220304486; 20220296159; 20220299619; 20220296127; 20220287632; 20220288125; 20220288312; 20220287622; 20220287631; U.S. Pat. No. 11,439,348; 20220280107; 20220280747; 20220273234; 20220277852; 20220273269; 20220273907; U.S. Pat. No. 11,426,079; 20220265207; 20220266014; 20220265223; 20220265206; 20220257176; 20220261020; 20220249017; 20220249429; 20220248882; 20220241530; 20220240843; 20220233102; 20220233119; 20220230522; 20220218779; 20220218274; 20220218988; 20220223294; 20220218273; U.S. Pat. No. 11,382,534; 20220192585; 20220192592; 20220192556; 20220183619; 20220183620; 20220183618; 20220176065; 20220160283; 20220160994; 20220160287; 20220152340; 20220155737; 20220151512; 20220148722; 20220142564; 20220133222; 20220133054; 20220134050; 20220138300; 20220133156; 20220134052; 20220126056; 20220125217; U.S. Pat. No. 11,305,110; 20220110949; 20220110547; 20220108797; 20220105308; 20220096401; 20220101975; 20220095993; U.S. Pat. No. 11,285,319; 20220088343; U.S. Pat. Nos. 11,278,724; 11,278,722; 20220079512; 20220084672; 20220079514; 20220071570; 20220071495; 20220071547; U.S. Pat. Nos. 11,266,346; 11,266,838; 20220062643; 20220066398; 20220061676; 20220061710; 20220054797; 20220054794; 20220059208; 20220054793; 20220054795; 20220059201; 20220054040; 20220059210; 20220032052; 20220031181; 20220031196; 20220031233; U.S. Pat. No. 11,235,148; 20220023584; 20220022809; 20220028511; 20220016386; and 20220015654.

Additionally, the data collected by the measuring device can be stored and analyzed over time to provide insights into the subject's sleep patterns and overall well-being. This information can be used to generate detailed sleep reports, offer recommendations for improving sleep hygiene, and further customize the device's settings to better meet the subject's needs.

The control unit preferably uses information from the measuring device to modulate and/or select the appropriate auditory stimuli. Modulation of the stimuli can involve adjusting various parameters such as volume, frequency, rhythm, and type of sound to match the detected sleep state. For example, gentle, slow-paced sounds might be used to promote relaxation and facilitate the transition into deeper sleep stages, while more rhythmic and structured sounds could be used to support REM sleep or to gently wake the subject.

The selection of stimuli can also be based on pre-determined settings or user preferences that are optimized according to the subject's physiological data. The control unit can offer a variety of auditory stimuli, including white noise, nature sounds, binaural beats, and calming music. The subject can manually select their preferred stimuli, or the control unit can automatically choose the most suitable stimuli based on the real-time input from the measuring device.

For instance, if the measuring device detects that the subject has entered a deep sleep stage, the control unit might reduce the volume or switch to a more soothing sound to maintain this state. Conversely, if the subject is in a light sleep stage or showing signs of restlessness, the control unit can increase the stimulus intensity or change the sound pattern to encourage a transition to a deeper sleep stage.

By preferably continuously adapting the auditory stimuli based on the subject's real-time physiological data, the device ensures that the stimuli remain effective and appropriate throughout the entire sleep cycle. This dynamic adjustment helps the subject achieve and maintain desired sleep states more efficiently, enhancing overall sleep quality.

The measuring device is a component designed to detect the sleep state of the subject. This device can either be a separate unit or integrated as part of the control unit.

The primary function of the measuring device is to monitor physiological parameters that indicate the subject's sleep state. By assessing physiological parameters, the measuring device advantageously provides real-time feedback to the control unit, allowing for dynamic adjustment of the auditory stimuli to better facilitate sleep.

The measuring device can be connected to the control unit via various means. It can utilize wired connections for reliable data transmission, ensuring that the control unit receives accurate and uninterrupted information about the subject's sleep state. Alternatively, the measuring device can employ wireless connections, such as Bluetooth or other radio frequency communications, to transmit data to the control unit. This wireless option provides greater flexibility and comfort for the subject, as it eliminates the need for physical cables.

When integrated into the control unit, the measuring device preferably shares a common housing and power source with the control unit, creating a compact and convenient solution. This integration can enhance the ease of use and portability of the device. As a separate unit, the measuring device can be placed in an optimal position to accurately monitor the subject's physiological signals, such as a wearable sensor or a bedside monitor.

In either configuration, the measuring device is designed to work seamlessly with the control unit, ensuring that the auditory stimuli are precisely tailored to the subject's current sleep state. This synergy between the measuring device and the control unit is critical for optimizing the effectiveness of the sleep induction process.

In another embodiment, the device is configured to prevent the subject from falling into certain avoidable or undesirable sleep states. This is exemplarily achieved by the measuring device detecting the onset of these sleep states and the control unit subsequently adjusting the auditory stimuli to either wake the subject up or shift the subject into another, for example safer, sleep state. This embodiment is particularly useful in scenarios where specific sleep states could be harmful to the subject, such as in cases of certain neurological conditions or to prevent sudden infant death syndrome (SIDS).

For example, in infants, the device can monitor for signs of sleep apnea or conditions that might lead to SIDS. If the device detects abnormal brain wave patterns or a prolonged period of apnea, the control unit can respond by adjusting the auditory stimuli to almost wake the infant up, thereby interrupting the dangerous sleep state and encouraging a shift to a safer sleep state.

Similarly, for individuals with certain neurological conditions, for example such as epilepsy and/or narcolepsy, specific sleep states may increase the likelihood of seizures or other unwanted conditions. The device can detect the onset of these high-risk sleep states and adjust the auditory stimuli to prevent the subject from entering them. This might involve increasing the volume or changing the nature of the stimuli to prompt the subject to shift to a different sleep state or wake up briefly.

This embodiment can also be useful in preventing sleep apnea. The device can detect periods of apnea and intervene by adjusting the auditory stimuli to encourage the subject to resume normal breathing patterns. This intervention can prevent prolonged apnea episodes and reduce associated risks such as oxygen deprivation.

For individuals with narcolepsy, the device can monitor for abnormal transitions between wakefulness and REM sleep. By adjusting the stimuli, the device can help manage sudden sleep attacks or episodes of cataplexy, ensuring the subject remains in a safe and manageable sleep state.

The ability to prevent the subject from falling into certain sleep states adds a significant safety feature to the device, making it particularly valuable for vulnerable populations such as infants and individuals with specific medical conditions. By leveraging real-time monitoring and adaptive auditory stimuli, this embodiment provides an effective tool for managing sleep-related health risks and ensuring a safer sleep environment.

In an embodiment of the device, the first and second transducers (e.g., headphones) are configured to emit independent tones from each other. This feature allows for the creation of complex auditory patterns, such as binaural beats, which can be particularly effective in inducing a sensation of motion and promoting relaxation.

The control unit in this configuration is adapted to generate and deliver distinct auditory stimuli to each transducer. For instance, a left headphone transducer might emit a tone at a specific frequency, while the right headphone transducer emits a tone at a slightly different frequency. This difference in frequency between the two headphones creates a phenomenon known as binaural beats, where the brain perceives a third tone that is the mathematical difference between the two frequencies.

Binaural beats have been shown to influence brain wave activity, encouraging states of relaxation, focus, or sleep, depending on the frequencies used. For sleep induction, the control unit can generate binaural beats that promote delta brain wave activity, which is associated with deep sleep. This personalized and dynamic auditory stimulation can enhance the effectiveness of the device in facilitating sleep.

Additionally, the ability to emit independent tones can allow for the creation of immersive and varied auditory experiences. The control unit can for example produce soundscapes that simulate natural environments or rhythmic movements, with each headphone contributing different elements of the sound. For example, one each may receive the sound of gentle waves, while the other may receive a soft, rhythmic hum, together creating a soothing and multidimensional auditory environment.

The independent tone emission capability can also support the device's adaptability to individual preferences and needs. Users can customize the auditory stimuli to their liking, selecting different combinations of tones and soundscapes that they find most relaxing. The control unit can store user preferences and adjust the stimuli accordingly, ensuring a consistently personalized sleep experience.

Furthermore, this embodiment enhances the device's ability to respond to real-time feedback from the measuring device. By dynamically adjusting the independent tones based on the detected sleep state and other physiological parameters, the control unit can fine-tune the auditory stimuli to optimize the sleep induction process continuously.

In an embodiment of the device, the audio transducers may comprise in-ear headphones, over-ear headphones, hearing aids, or headphones designed to be worn in a manner not directly on the head or ears of the subject. In another embodiment, spatialized audio is used to direct distinct audio signals to each ear, and may comprise an array of acoustic transducers, such as 8 or 16 transducers (speakers).

The headphones are preferably of various types and configurations to suit the needs and preferences of the subject. In-ear headphones preferably fit snugly within the ear canal, providing a compact and portable option, while over-ear headphones preferably cover the entire outer ear, often providing better sound isolation and comfort for extended use.

The audio transducers can also be configured, with the transducers placed in the vicinity of the subject, such as integrated into a pillow, headrest, or positioned around a bed area. This flexibility in design ensures that the auditory stimuli can be delivered effectively without causing discomfort or inconvenience to the user.

The transducers can possess the capability to filter out or cancel ambient sounds from the environment surrounding the subject. This sound filtering or cancelling can be achieved through active and/or passive noise filtering methods. Active noise filtering generally involves the use of electronics to generate sound waves that cancel out ambient noise, while passive noise filtering usually relies on the physical design and materials of the headphones to block and/or minimize external sounds.

The audio transducers can be connected to the control unit via wireless or wired connections. Wireless connections can include technologies such as Bluetooth or other radio frequency communications, providing the subject with greater mobility and convenience. Alternatively, wired connections ensure a stable and uninterrupted link between the audio transducer or transducers and the control unit, which can be beneficial in certain environments.

In an embodiment, the measuring device is designed to either process the collected data to evaluate the sleep state of the subject and transmit this information directly to the control unit, referred as a first configuration, or alternatively, to transmit the raw data to the control unit where the evaluation is then performed, referred as a second configuration.

In the first configuration, the measuring device comprises processing capabilities that allow it to analyze the physiological data it collects. The measuring device preferably uses built-in algorithms to interpret this data and determine the subject's current sleep state. Once the sleep state is determined, the measuring device transmits this specific information to the control unit. This approach allows for quick and efficient adjustments to the auditory stimuli since the control unit receives ready-to-use sleep state information.

Alternatively, in the second configuration, the measuring device transmits the raw data it collects directly to the control unit. In this configuration, the control unit is equipped with processing capabilities and/or algorithms to analyze this raw data, preferably in real-time (i.e., the processing and data output keep pace with the input data, with some latency). By evaluating the incoming data, the control unit determines the subject's sleep state and adjusts the auditory stimuli accordingly. This method leverages the central processing power of the control unit to handle data analysis, potentially allowing for more advanced and adaptable algorithms that can be updated or improved over time. The algorithm may be adaptive and/or learning.

In an embodiment, the measuring device comprises an EEG (electroencephalogram) sensor adapted to monitor the brain waves of the subject. The EEG sensor is preferably designed to detect and/or record the electrical activity of the subject's brain, preferably providing real-time feedback on the subject's sleep state and brain wave patterns.

See, U.S. Patent and Pub. Pat. Application Nos. U.S. Pat. No. 12,029,573; 20240186007; 20240168553; 20240157146; 20240148324; 20240143348; 20240134868; 20240108263; 20240108511; 20240074700; 20240050762; 20240050021; 20240023892; U.S. Pat. No. 11,875,656; 20240005398; 20230404466; 20230390147; 20230356095; 20230344907; 20230338136; 20230277851; 20230270367; 20230256191; 20230240584; 20230211239; 20230190185; 20230190196; 20230178206; 20230162851; 20230130186; U.S. Pat. No. 11,622,709; 20230082594; 20230073174; 20230051757; 20230039882; 20230031613; 20230008214; 20220409117; 20220391015; 20220387749; 20220366459; 20220331196; 20220331197; 20220323290; 20220313135; 20220313541; 20220319477; U.S. Pat. No. 11,457,867; 20220241474; 20220240846; 20220233119; 20220183611; 20220180993; 20220175303; 20220160291; 20220160309; 20220133194; 20220117837; 20220118258; 20220111212; 20220095920; 20220101981; 20220061735; 20210399911; 20210400142; U.S. Pat. No. 11,204,989; 20210391083; 20210373676; 20210374391; 20210338973; 20210307673; 20210290890; U.S. Pat. No. 11,128,636; 20210259557; 20210169417; 20210162148; 20210118547; 20210106490; 20210100491; 20210041953; 20210023330; 20200390337; 20200367789; 20200368491; 20200364539; 20200365125; 20200353933; 20200345287; U.S. Pat. No. 10,820,817; 20200337625; 20200307437; 20200253527; 20200245933; 20200225749; 20200222276; 20200187841; 20200187829; 20200178875; 20200178833; U.S. Pat. Nos. 10,660,575; 10,656,710; 20200085312; 20200082735; 20200060566; U.S. Pat. No. 10,564,717; 20200037942; 20200008725; 20190387998; 20190380597; 20190378621; 20190307351; U.S. Pat. No. 10,430,557; 20190290211; 20190290157; 20190294244; 20190227626; 20190200925; 20190192077; 20190171348; 20190174039; U.S. Pat. No. 10,285,646; 20190113973; 20190053766; 20190046044; 20190000338; 20180348863; D835287; 20180336970; 20180326999; 20180310855; U.S. Pat. Nos. 10,109,216; 10,098,582; 20180279946; U.S. Pat. No. 10,076,279; 20180241483; 20180204266; 20180184964; 20180184962; 20180189678; 20180154860; 20180110669; 20180103903; 20180103913; 20180103894; 20180104439; 20180092548; 20180092547; 20180092574; 20180092549; 20180092588; 20180092589; 20180094982; 20180096198; 20180092542; 20180049896; 20170354341; 20170311832; U.S. Pat. No. 9,776,043; 20170258390; 20170251985; 20170229037; 20170228512; 20170202475; 20170197086; 20170188976; U.S. Pat. No. 9,685,174; 20170171441; 20170143228; 20170123495; 20170113641; 20170113702; 20170080234; 20170071551; 20170055903; 20170056642; 20170039045; 20170007165; 20170004260; U.S. Pat. No. 9,507,974; 20160300252; U.S. Pat. Nos. 9,445,739; 9,418,368; 20160224803; 20160170996; 20160170998; 20160171514; U.S. Pat. No. 9,336,535; 20160055236; 20160022206; 20150351655; 20150323986; 20150313496; 20150297109; 20150282760; 20150257673; 20150199010; 20150150753; 20150145682; 20150091791; 20150079578; 20150045007; 20150045688; 20140347265; 20140342326; 20140316230; 20140276183; 20140277582; 20140223462; 20140221866; 20140200432; 20140051961; U.S. Pat. No. 8,483,816; 20110298706; and 20100076334.

The EEG sensor can be integrated into a headband, cap, or for example in another wearable device that the subject can comfortably wear during sleep. This wearable device may be designed to be non-intrusive and lightweight, ensuring that it does not interfere with the subject's ability to fall asleep or remain asleep.

The primary function of the EEG sensor is to monitor brain wave activity, which can include various frequency ranges such as delta, theta, alpha, and beta waves. By analyzing brain waves, the EEG sensor can determine the different stages of sleep the subject is experiencing, including light sleep, deep sleep, and REM (rapid eye movement) sleep.

For instance, if the EEG sensor data shows that the subject is in a state of light sleep, the control unit can deliver auditory stimuli designed to promote deeper sleep, such as binaural beats that encourage delta wave activity. Conversely, if the sensor detects that the subject is in a state of deep sleep, the control unit can maintain or reduce the intensity of the stimuli to avoid unnecessary disruptions.

By monitoring brain wave activity, the device can make precise adjustments to the auditory stimuli to optimize the sleep environment for the subject. This ensures that the stimuli remain effective and appropriate throughout the entire sleep cycle.

Additionally, the data collected by the EEG sensor can be stored and analyzed over time to provide insights into the subject's sleep patterns and quality. This information can be used to generate detailed sleep reports, offer recommendations for improving sleep hygiene, and further customize the device's settings to better meet the subject's needs.

In another embodiment, the measuring device comprises an EOG (electrooculogram) sensor adapted to detect the eye movements of the subject. This sensor is preferably designed to monitor and record the electrical activity generated by eye movements, which is indicative of different sleep stages, particularly the REM (rapid eye movement) stage of sleep.

See, U.S. Patent and Pub. Pat. Application Nos. 20240188868; 20240169208; 20240012476; U.S. Pat. Nos. 11,684,305; 11,507,185; 11,298,064; 20220022805; 20200330017; 20200185159; 20200178895; 20200085369; 20200081247; U.S. Pat. No. 10,564,717; 20190320891; 20190155226; 20180368722; 20180014784; 20170150897; 20150126845; 20140303428; 20140221779; 2012020375; 20060061544; and 20020188216.

The EOG sensor can be integrated into a wearable device, such as an eye mask or headband, that the subject wears during sleep. This wearable device is preferably designed to be comfortable and non-intrusive, allowing the subject to wear it throughout the night without any discomfort.

The EOG sensor is preferably configured to detect and differentiate between various types of eye movements. For instance, slow rolling eye movements are typically associated with the transition into light sleep, while rapid eye movements are characteristic of the REM stage of sleep. By continuously monitoring these eye movements, the EOG sensor can provide real-time data on the subject's sleep stages.

For example, if the EOG data shows that the subject is entering REM sleep, the control unit can adjust the auditory stimuli to maintain an optimal sleep environment. This might involve playing sounds that are conducive to REM sleep, such as gentle, rhythmic patterns that do not disrupt the rapid eye movements. Conversely, if the sensor detects that the subject is transitioning out of REM sleep into a lighter sleep stage, the control unit can modify the stimuli to support this transition smoothly.

This embodiment allows for a highly personalized and adaptive sleep induction process. By continuously monitoring eye movements, the device can make precise adjustments to the auditory stimuli to optimize the sleep environment for the subject. This ensures that the stimuli remain effective and appropriate throughout the entire sleep cycle.

Additionally, the data collected by the EOG sensor can be stored and analyzed over time to provide insights into the subject's sleep patterns and quality. This information can be used to generate detailed sleep reports, offer recommendations for improving sleep hygiene, and further customize the device's settings to better meet the subject's needs.

The sleep state of a person may be monitored using a radar sensor (e.g., Project Soli), WiFi radar, camera (video camera), hyperspectral camera, or other electromagnetic sensor. The sensor may detect breath interval, heart beat interval (and derivative measurements variability and rate), movement, photoplethysmography, or the like.

See, U.S. Patent and Pub. Pat. Application Nos. 20240225454; 20240188905; 20240130621; 20240015445; 20230376581; 20230363654; U.S. Pat. No. 11,793,914; 20230210443; 20230190140; 20230157757; 20230157762; 20230125188; 20230107915; U.S. Pat. No. 11,614,511; 20230023083; 20230000396; 20220370752; 20220373646; 20220361954; 20220281117; 20220286885; 20220233860; 20220131424; 20220105307; 20220083120; U.S. Pat. No. 11,207,025; 20210378532; 20210369394; 20210345939; 20210338174; U.S. Pat. No. 11,156,965; 20210315464; 20210318558; U.S. Pat. Nos. 11,141,063; 11,141,129; 11,116,448; 20210138232; 20210113130; U.S. Pat. No. 10,918,291; 20210022624; 20200368527; 20200345274; 20200306496; 20200236545; 20200187916; 20200184153; 20200121214; 20200121215; U.S. Pat. No. 10,610,145; 20190274553; 20190274547; 20190259108; 20190239850; 20190182415; U.S. Pat. No. 10,300,370; 20190154439; U.S. Pat. No. 10,241,581; 20190046120; U.S. Pat. No. 10,175,781; U.S. Pat. No. 10,085,683; 20180263220; U.S. Pat. Nos. 10,058,284; 9,993,166; 20180102035; U.S. Pat. No. 9,837,760; 20170173262; U.S. Pat. Nos. 9,579,060; 9,554,738; 20150363566; 20150287422; 20150151142; U.S. Pat. No. 8,983,493; 20150068069; 20150031964; 20140359722; 20140194793; 20140142403; 20140135631; 20140125619; 20140079248; 20130324788; 20130300573; 20130173926; U.S. Pat. No. 8,478,297; 20130030257; 20120272738; 20120274498; 20120274502; 20120274503; 20120276849; 20120245439; 20120146796; 20120123232; 20120022348; 20110251021; 20110130800; 20110077526; 20100286527; 20100245091; 20100152600; 20090005679; 20080294019; 20080188752; 20080146943; 20080146940; 20080146925; 20080114239; 20080110261; 20080110266; 20080114241; 20080114247; 20080114248; 20080114249; 20080114253; 20080114255; 20080112265; 20080114245; 20080114246; 20080114252; 20080001735; 20070161904; and 20060106289

In another embodiment, the measuring device comprises a pulse oximeter adapted to measure the blood oxygen levels and/or heart rate variability of the subject.

The pulse oximeter (one example of a photoplethysmography can be integrated for example into a wearable device such as a wristband, fingertip sensor, or sensor) earlobe clip. This wearable device is preferably designed to be comfortable and non-intrusive, allowing the subject to wear it continuously throughout the night without any discomfort or inconvenience.

See, U.S. Patent and Pub. Pat. Application Nos. 20240225532; 20240225466; 20240227260; 20240225542; 20240225540; 20240226482; 20240230680; 20240225496; U.S. Pat. Nos. 12,029,434; 12,029,940; 12,030,818; 12,033,749; 12,029,536; 12,033,423; 20240215908; 20240219237; 20240211566; 20240211719; 20240207656; 20240207554; 20240207566; 20240206810; 20240206804; 20240197215; 20240197188; 20240203586; 20240203558; 20240203585; 20240203602; 20240197199; 20240197249; 20240194346; 20240194343; 20240194344; 20240194353; 20240188876; U.S. Pat. No. 12,010,770; 20240180489; 20240181185; 20240185997; 20240180522; 20240180498; 20240180484; 20240180774; 20240181277; U.S. Pat. No. 11,998,281; 20240173499; 20240164678; 20240164724; 20240156355; 20240156377; 20240156538; 20240156388; 20240157123; 20240148289; 20240148331; 20240145085; 20240138759; 20240139446; 20240139448; 20240131287; 20240131364; U.S. Pat. Nos. 11,963,746; 11,963,764; 20240127954; 20240127966; 20240122500; 20240123223; 20240122535; 20240123227; 20240122550; 20240122544; 20240122548; 20240115414; U.S. Pat. Nos. 11,950,891; 11,954,862; 11,950,851; 20240114282; 20240111253; 20240109553; 20240108242; 20240105335; 20240106283; 20240099593; 20240090827; 20240090808; 20240091437; 20240098168; 20240098921; 20240096463; 20240094777; 20240094821; U.S. Pat. No. 11,931,118; 20240081696; 20240081657; 20240081655; 20240081647; U.S. Pat. No. 11,925,441; 20240075225; 20240074701; 20240074709; 20240079763; 20240081012; 20240080998; 20240077526; 20240075320; 20240074703; U.S. Pat. No. 11,918,310; 20240065925; 20240066249; 20240071597; 20240071624; 20240065764; 20240065550; U.S. Pat. Nos. 11,911,174; 11,911,181; 11,914,842; 20240062870; 20240057181; U.S. Pat. Nos. 11,896,329; 11,896,328; 20240041342; 20240042149; 20240041401; 20240041340; 20240041331; U.S. Pat. No. 11,894,126; 20240038343; 20240032853; 20240032825; 20240032836; 20240024597; 20240031460; 20240016411; 20240016447; 20240016397; 20240016431; 20240016413; 20240016654; 20240008814; 20240013918; 20240007777; 20240005432; 20240001068; 20240000324; 20240000349; 20240006067; 20240008205; 20240005398; 20240000328; 20240000204; 20240000205; 20240000206; 20230418421; 20230404444; 20230410636; 20230405374; 20230410976; U.S. Pat. No. 11,844,593; 20230397880; 20230402185; 20230397851; 20230397852; 20230390513; 20230389848; 20230389870; 20230380758; 20230380692; 20230380776; 20230371894; 20230377114; 20230371870; 20230375525; 20230377543; 20230371826; 20230377712; 20230371890; 20230372284; U.S. Pat. No. 11,823,037; 20230363653; 20230363700; 20230363655; 20230367833; 20230368887; 20230363712; 20230364365; 20230355875; 20230355145; 20230360765; 20230355177; 20230352141; 20230346237; 20230337937; 20230338691; 20230343451; 20230341944; 20230343458; 20230338677; 20230329636; 20230330378; 20230336928; U.S. Pat. Nos. 11,790,586; 11,786,313; 20230321436; 20230320602; U.S. Pat. No. 11,779,235; 20230309866; 20230317218; 20230309909; 20230309854; 20230319453; U.S. Pat. Nos. 11,775,026; 11,771,350; 20230301523; U.S. Pat. No. 11,766,196; 20230293029; 20230298761; U.S. Pat. No. 11,762,342; 20230290511; 20230284726; 20230284905; 20230277068; 20230277122; 20230277069; 20230277062; 20230284022; 20230283848; 20230282363; 20230277056; 20230277074; 20230277123; 20230277410; 20230270350; 20230270967; 20230270360; 20230270387; 20230270562; 20230263400; 20230263459; U.S. Pat. No. 11,730,916; 20230255527; 20230255519; 20230253103; 20230248609; 20230240546; 20230241337; 20230240591; 20230240531; 20230240603; U.S. Pat. Nos. 11,712,190; 11,712,176; 20230233089; 20230237897; U.S. Pat. No. 11,707,208; 20230232558; 20230225676; 20230218845; 20230218844; 20230218225; 20230218907; 20230210443; 20230214470; 20230215554; 20230215557; 20230215560; 20230210464; 20230210468; 20230210395; 20230210400; 20230210385; 20230210444; 20230200668; 20230200749; 20230200746; 20230200663; 20230200751; U.S. Pat. No. 11,684,295; 20230190140; 20230190125; 20230191052; 20230190124; 20230190120; 20230190197; 20230190201; 20230190206; 20230197265; 20230199365; U.S. Pat. No. 11,678,812; 20230181869; 20230181112; 20230181258; U.S. Pat. No. 11,672,614; 20230172499; 20230173221; 20230172486; U.S. Pat. No. 11,670,410; 20230165539; 20230170087; 20230165506; 20230168740; 20230165508; 20230165524; U.S. Pat. No. 11,660,005; 20230157647; 20230158268; 20230157646; 20230157632; 20230148952; 20230149711; 20230148886; 20230149746; 20230147605; 20230144761; 20230141079; 20230140151; U.S. Pat. No. 11,638,532; 20230127355; U.S. Pat. No. 11,633,147; 20230111348; 20230114269; 20230114833; 20230113714; 20230115575; 20230112231; U.S. Pat. Nos. 11,622,723; 11,622,717; 20230108409; 20230104416; 20230107454; 20230105636; U.S. Pat. Nos. 11,620,497; 11,617,520; 20230098734; 20230095971; 20230096307; 20230090541; 20230091853; 20230086651; U.S. Pat. No. 11,607,152; 20230078228; 20230085305; 20230082016; 20230084205; 20230080356; 20230081794; 20230081715; U.S. Pat. No. 11,605,038; 20230071085; 20230075570; 20230072281; 20230076069; 20230075634; 20230071467; 20230072872; U.S. Pat. No. 11,596,361; 20230069040; 20230068534; 20230065695; 20230062794; 20230061823; 20230066808; 20230054041; 20230058052; 20230054209; 20230055617; 20230056403; 20230057961; 20230058011; 20230059343; 20230059947; U.S. Pat. No. 11,583,361; 20230046769; 20230048327; U.S. Pat. No. 11,576,579; 20230042347; 20230037360; 20230039882; 20230040540; U.S. Pat. No. 11,571,266; 20230031613; 20230036913; 20230030688; 20230036114; 20230033976; 20230030071; 20230029042; 20230028745; 20230012507; 20230014168; 20230020039; 20230010015; U.S. Pat. No. 11,547,486; 20230000442; 20230000353; 20230000714; U.S. Pat. Nos. 11,545,263; 11,543,292; 20220409186; 20220409847; 20220409850; 20220414181; 20220415476; 20220413143; 20220409187; 20220402502; 20220406467; 20220408199; 20220400989; 20220400967; 20220401037; 20220395192; 20220386959; 20220387158; U.S. Pat. Nos. 11,510,584; 11,510,733; 11,515,044; 20220370010; 20220375590; 20220375591; 20220368777; 20220361810; 20220361823; U.S. Pat. No. 11,497,436; 20220354418; 20220351852; 20220343517; 20220338745; 20220344055; 20220344057; 20220330854; 20220330821; 20220330885; 20220331065; U.S. Pat. Nos. 11,464,573; 11,464,589; 11,464,451; 11,464,650; 11,464,590; 20220313100; 20220319690; 20220313087; 20220313945; U.S. Pat. Nos. 11,457,983; 11,462,324; 20220304590; U.S. Pat. No. 11,452,470; 20220301666; 20220296169; 20220296105; 20220287592; 20220287572; 20220287579; 20220288398; 20220287622; 20220280072; 20220279266; 20220265150; 20220268229; 20220269090; 20220270344; U.S. Pat. Nos. 11,419,530; 11,419,529; 20220257460; 20220249020; 20220256294; 20220249055; 20220249002; 20220256720; 20220241670; 20220240790; 20220240832; 20220233860; 20220233123; 20220233096; 20220233114; 20220225929; 20220225887; 20220229895; 20220225901; 20220225945; U.S. Pat. Nos. 11,389,092; 11,389,248; 11,375,926; 20220202350; 20220192549; 20220197984; 20220192596; U.S. Pat. Nos. 11,363,990; 11,364,380; 11,363,996; 20220183606; 20220190188; 20220186090; U.S. Pat. No. 11,357,582; 20220176088; 20220175324; 20220176057; 20220160308; 20220160309; and 20220160311.

The pulse oximeter is preferably configured to measure the oxygen saturation levels in the subject's blood (SpO₂) and/or to monitor heart rate variability (HRV). Blood oxygen levels provide critical information about the subject's respiratory function, while heart rate variability is a key indicator of autonomic nervous system activity. Blood oxygen levels and heart rate variability are key indicators of the subject's sleep states. Blood oxygen levels provide critical information about the subject's respiratory function, while heart rate variability reflects the activity of the autonomic nervous system and is closely associated with different stages of sleep.

Additionally, if the pulse oximeter indicates to low blood oxygen levels, which might indicate respiratory issues such as sleep apnea, the control unit can respond by adjusting the auditory stimuli to prompt the subject to change their position or to wake up slightly to resume normal breathing. Similarly, if the heart rate variability indicates high stress or discomfort, the control unit can deliver calming auditory stimuli designed to promote relaxation and improve heart rate variability.

Additionally, the data collected by the pulse oximeter can be stored and analyzed over time to provide insights into the subject's sleep patterns, respiratory health, and overall well-being. This information can be used to generate detailed health reports, offer recommendations for improving sleep hygiene and respiratory function, and further customize the device's settings to better meet the subject's needs.

In another embodiment, the measuring device comprises a temperature sensor adapted to measure the body and/or skin temperature of the subject.

The temperature sensor can be integrated into a wearable device such as a wristband, patch, or headband, designed to be comfortable and non-intrusive for the subject to wear throughout the night. Alternatively, the sensor can be incorporated into the sleeping environment, such as embedded in the mattress, bedding, or pillow.

The primary function of the temperature sensor is to monitor the body and/or skin temperature of the subject. Body temperature is a key indicator of the circadian rhythm and sleep stages. Typically, the body's core temperature decreases during the initial stages of sleep and reaches its lowest point during deep sleep. By tracking these temperature variations, the temperature sensor provides insights into the subject's sleep state.

For example, if the temperature sensor detects that the subject's body temperature is rising, which may indicate the subject is transitioning from deep sleep to a lighter sleep stage or waking up, the control unit can modify the auditory stimuli to maintain or enhance the sleep state. Conversely, if the sensor detects a stable, low body temperature indicating deep sleep, the control unit can minimize disruptions by maintaining consistent, soothing auditory stimuli.

Additionally, the data collected by the temperature sensor can be stored and analyzed over time to provide insights into the subject's sleep patterns and overall thermal comfort. This information can be used to generate detailed sleep reports, offer recommendations for improving sleep hygiene, and further customize the device's settings to better meet the subject's needs.

In another embodiment, the measuring device comprises a movement sensor adapted to detect body movements, breathing rate, and/or position changes of the subject. This embodiment enhances the device's ability to monitor physiological parameters that are indicative of the subject's sleep states and overall sleep quality.

The movement sensor can include an accelerometer for actigraphy, which is capable of detecting and recording body movements. Reduced movement generally indicates deeper stages of sleep. The accelerometer can be integrated into a wearable device such as a wristband, or it can be embedded in the sleeping environment, such as within the mattress or bedding.

In addition or alternatively to monitoring movements, the device can also measure breathing patterns using respiratory belts or other suitable sensors. These sensors are designed to detect the expansion and contraction of the chest and/or abdomen, providing data on the subject's breathing rate and rhythm. Monitoring breathing patterns enables identifying sleep stages, as changes in respiration often correspond with transitions between different stages of sleep.

The movement sensor is preferably also capable of detecting position changes. This information is valuable for understanding the subject's sleep behavior and can help in identifying issues such as restless leg syndrome or frequent position shifts that might disrupt sleep.

The primary function of these sensors is to provide comprehensive data on the subject's physical activity and physiological states throughout the sleep cycle.

The control unit processes the data received from the movement sensor to adjust the auditory stimuli accordingly. For example, if the sensor detects minimal movement and a stable breathing pattern, indicative of deep sleep, the control unit might reduce the auditory stimuli to maintain this restful state. Conversely, if increased movement or irregular breathing patterns are detected, suggesting lighter sleep or potential disturbances, the control unit can modify the stimuli to promote deeper, more restorative sleep.

This embodiment allows for a highly personalized and adaptive sleep induction process. By continuously monitoring body movements, breathing rate, and position changes, the device can make precise adjustments to the auditory stimuli to optimize the sleep environment for the subject.

Additionally, the data collected by the movement sensor can be stored and analyzed over time to provide insights into the subject's sleep patterns, respiratory health, and overall sleep behavior.

In another embodiment, the measuring device comprises a microphone configured to analyze breathing, snoring patterns, and/or other auditory cues indicative of the sleep stages of the subject. This embodiment enhances the device's ability to monitor auditory signals that provide valuable insights into the subject's sleep quality and physiological state.

The microphone can be integrated into a wearable device, such as a headband, or positioned in the sleeping environment, such as placed on a bedside table or embedded in the bedframe. The design ensures that the microphone is in close proximity to the subject to accurately capture the necessary sounds without being intrusive or causing discomfort.

The primary function of the microphone is to detect various auditory signals produced by the subject during sleep. Breathing patterns can indicate different stages of sleep, with slow, regular breathing typically associated with deep sleep and irregular breathing patterns often observed during REM sleep. Snoring patterns can provide information about potential sleep disturbances such as sleep apnea. Additionally, other auditory cues, such as movement-related sounds or sleep talking, can be indicators of sleep stage transitions or disruptions.

For instance, if the microphone detects a consistent snoring pattern that suggests obstructive sleep apnea, the control unit can adjust the auditory stimuli to prompt a position change or slight awakening to alleviate the condition. If the microphone detects regular, deep breathing, indicative of deep sleep, the control unit can minimize the stimuli to maintain this restorative stage.

This embodiment allows for a highly personalized and adaptive sleep induction process. By continuously monitoring auditory cues, the device can make precise adjustments to the auditory stimuli to optimize the sleep environment for the subject. This ensures that the stimuli remain effective and appropriate throughout the entire sleep cycle.

Additionally, the data collected by the microphone can be stored and analyzed over time to provide insights into the subject's sleep patterns and overall sleep quality. This information can be used to generate detailed sleep reports, offer recommendations for improving sleep hygiene, and further customize the device's settings to better meet the subject's needs.

In another embodiment, the measuring device comprises a pressure sensor adapted to detect changes in pressure on a surface where the subject is positioned. This embodiment allows to monitor physical interactions with the sleeping surface, which are indicative of sleep stages and movements of the subject.

The pressure sensor can for example be integrated into the mattress, pillow, or bedding, ensuring that it is in close contact with the subject to accurately capture pressure changes. This pressure sensor is preferably designed to be highly sensitive, detecting even minor adjustments in the subject's position and movements. The primary function of the pressure sensor is to monitor changes in pressure that correlate with the subject's movements and sleep stages. For example, reduced movement and stable pressure readings are generally associated with deeper stages of sleep, while frequent pressure changes might indicate lighter sleep stages or restlessness. By continuously tracking these pressure variations, the sensor provides valuable data on the subject's sleep quality and behavior.

In another embodiment, the measuring device comprises sensors adapted to detect galvanic skin response (GSR), which measures changes in skin conductivity. These changes in skin conductivity are often indicative of emotional arousal or stress levels, which can be connected with sleep quality and stages.

The GSR sensors can for example be integrated into a wearable device, such as a wristband or headband, designed to be comfortable for the subject to wear throughout the night. These sensors continuously monitor the electrical conductance of the skin, providing real-time data on the subject's physiological state. This data is transmitted to the control unit, which processes the information to adjust the auditory stimuli accordingly. For example, if the GSR sensors detect high stress levels, the control unit can modify the stimuli to promote relaxation and facilitate the transition to deeper sleep stages.

See, U.S. Patent and Pub. Pat. Application Nos. 20240233029; 20240234654; 20240226477; U.S. Pat. Nos. 12,029,852; 12,029,573; 12,033,751; 12,032,730; 20240215908; U.S. Pat. No. 12,023,382; 20240206812; 20240206807; 20240211566; 20240211719; 20240207554; 20240207566; 20240206810; 20240196863; 20240203558; 20240203602; 20240194343; 20240194344; 20240188682; 20240184361; 20240181185; 20240173499; 20240164675; 20240145085; 20240145525; 20240144721; 20240139446; 20240139448; 20240130661; 20240126218; 20240127927; 20240114282; 20240112562; 20240109553; 20240108148; 20240108242; 20240106283; 20240103285; 20240090807; 20240091476; 20240094556; 20240090818; 20240075225; 20240074709; U.S. Pat. No. 11,918,379; 20240066249; 20240065550; 20240062872; 20240042149; 20240033457; 20240033459; 20240040299; 20240037854; 20240038343; 20240024597; 20240029109; 20240016447; 20240008783; 20240015445; 20240013256; 20240009416; 20240008775; 20240007777; 20240000344; U.S. Pat. No. 11,857,340; 20230417544; 20230420124; 20230405250; 20230411018; 20230410636; 20230397880; 20230400327; U.S. Pat. No. 11,832,934; 20230380758; 20230380763; 20230371837; 20230377114; U.S. Pat. No. 11,822,719; 20230364368; 20230363700; 20230363670; 20230364365; 20230364378; 20230359152; 20230356028; 20230361453; 20230355177; 20230346221; 20230343435; 20230338677; 20230335287; 20230335257; 20230335253; 20230328417; 20230325006; 20230310781; 20230309933; U.S. Pat. No. 11,762,457; 20230291952; 20230284754; 20230285808; 20230277123; 20230270350; 20230270378; 20230270387; U.S. Pat. Nos. 11,740,853; 11,741,517; 20230263482; 20230253103; 20230248927; 20230249599; 20230248258; 20230240595; 20230245780; 20230241337; 20230233158; 20230226308; 20230218845; 20230218221; 20230218844; U.S. Pat. No. 11,696,682; 20230210443; 20230206486; 20230207100; 20230200673; 20230209355; 20230190140; 20230191063; 20230191053; 20230190167; 20230190193; U.S. Pat. No. 11,678,812; 20230185397; 20230181869; 20230181121; 20230173221; 20230172486; 20230165498; 20230161384; 20230157636; 20230148952; 20230148863; 20230144677; U.S. Pat. Nos. 11,642,038; 11,642,039; 20230136939; 20230128051; 20230118067; 20230111477; 20230112797; U.S. Pat. No. 11,622,717; 20230107369; 20230106450; 20230099622; 20230098734; 20230096515; 20230085305; 20230079489; 20230074565; 20230066862; 20230059015; 20230056779; 20230048000; 20230048912; 20230046769; 20230047787; 20230037360; 20230037675; 20230038068; 20230037749; U.S. Pat. No. 11,571,048; 20230032131; 20230036312; 20230033838; 20230032290; U.S. Pat. No. 11,570,869; 20230008561; U.S. Pat. No. 11,551,803; 20230000396; 20230000428; 20230000353; 20220409847; 20220409041; 20220401673; 20220406473; 20220402502; 20220408199; 20220402517; 20220395222; 20220398775; 20220386915; 20220392354; 20220392237; 20220387158; 20220370752; 20220377542; 20220371605; 20220369975; 20220362095; 20220357707; 20220346682; 20220347412; 20220351856; 20220339380; 20220338743; 20220331606; 20220327956; U.S. Pat. No. 11,461,952; 20220310247; 20220304973; 20220296168; 20220300914; 20220280105; U.S. Pat. No. 11,435,826; 20220273269; U.S. Pat. No. 11,419,509; 20220257186; 20220248980; 20220249032; 20220244032; 20220233139; 20220224963; 20220218198; 20220219085; 20220202373; 20220210586; 20220192592; 20220198903; 20220197986; 20220198206; 20220192530; 20220183618; 20220190188; U.S. Pat. No. 11,347,189; 20220160274; 20220160265; 20220160309; 20220157436; 20220139252; 20220125217; 20220121283; 20220117558; 20220114616; 20220113798; 20220113799; 20220115010; 20220115137; 20220109705; 20220100273; 20220096010; 20220096007; 20220101873; U.S. Pat. No. 11,288,977; 20220087615; 20220075450; 20220075857; U.S. Pat. No. 11,263,749; 20220051567; 20220040418; 20220036481; U.S. Pat. No. 11,238,664; 20220028529; 20220028541; 20220020461; 20220015675; 20220017077; U.S. Pat. Nos. 11,228,810; 11,227,040; 11,223,631; 20210406983; 20210398562; 20210399911; 20210400142; 20210398648; 20210390366; 20210386306; 20210381839; 20210379388; U.S. Pat. No. 11,195,615; 20210373230; 20210374391; 20210345926; 20210345894; 20210338948; 20210342020; 20210315633; 20210309252; 20210298622; U.S. Pat. No. 11,128,636; 20210284267; 20210274754; 20210275102; 20210275034; 20210280308; 20210280322; 20210259557; 20210260441; 20210257086; 20210247861; 20210241553; 20210233204; 20210225374; 20210217533; 20210208753; 20210208624; 20210208841; 20210188332; 20210188291; 20210183477; 20210177342; 20210185460; 20210169406; 20210169417; 20210174952; 20210174933; 20210173480; 20210161423; 20210168450; 20210158927; 20210150669; 20210140671; 20210110838; 20210106238; 20210096646; 20210097408; U.S. Pat. Nos. 10,963,964; 10,958,466; 20210068736; U.S. Pat. Nos. 10,939,866; 10,943,407; 20210060384; 20210057092; 20210038141; 20210043011; 20210031786; 20210030308; 20210034727; 20210035431; 20210015417; 20210007669; 20210008332; 20210008331; 20210000376; 20210004715; 20210000347; 20200405213; 20200405158; U.S. Pat. No. 10,877,444; 20200402641; 20200401933; 20200390402; 20200390390; 20200390343; 20200383581; 20200383633; U.S. Pat. No. 10,856,803; 20200376324; 20200371738; 20200367824; 20200364539; 20200359914; 20200356171; 20200350057; 20200351657; U.S. Pat. No. 10,825,564; 20200337567; 20200334479; 20200331481; 20200324104; 20200319472; 20200306493; 20200307610; 20200314518; 20200314490; 20200297955; 20200297223; 20200296480; 20200294670; 20200286505; 20200285069; 20200278556; 20200279035; 20200268260; 20200267936; 20200268263; 20200265744; 20200253473; 20200259638; 20200251213; 20200249752; 20200237297; 20200234813; 20200225963; 20200214617; 20200218312; 20200219615; 20200217727; 20200206631; 20200204390; U.S. Pat. Nos. 10,692,606; 10,687,756; 20200196327; 20200178885; 20200175255; 20200175873; U.S. Pat. No. 10,667,714; 20200163966; 20200163616; 20200167798; 20200146625; 20200146397; 20200153624; 20200146563; 20200143286; 20200143085; 20200138356; 20200137357; 20200130674; U.S. Pat. No. 10,631,727; 20200128232; 20200126402; U.S. Pat. No. 10,631,029; 20200114150; 20200117690; 20200100693; 20200085362; 20200085311; U.S. Pat. No. 10,594,689; 20200073478; 20200059703; 20200057487; 20200051447; 20200037945; 20200043594; 20200027378; 20200026272; 20200028687; 20200023157; 20200020165; 20200005663; 20200008299; U.S. Pat. No. 10,523,852; 20190388794; 20190387998; 20190392441; 20190380661; U.S. Pat. No. 10,509,466; 20190374150; U.S. Pat. No. 10,503,970; 20190369726; 20190370299; 20190366558; 20190360051; 20190361917; 20190354181; 20190351912; 20190350526; U.S. Pat. No. 10,482,333; 20190349367; 20190336062; 20190332902; 20190324551; 20190313916; 20190313947; 20190306550; 20190306613; 20190292600; 20190283247; 20190283394; 20190274623; U.S. Pat. No. 10,409,545; 20190269328; U.S. Pat. No. 10,405,034; 20190261874; 20190250408; 20190247650; 20190239431; 20190239969; 20190239806; 20190231257; 20190232047; 20190231235; 20190228439; 20190224481; 20190227626; 20190221059; 20190215030; 20190213429; U.S. Pat. No. 10,349,148; 20190208414; 20190197757; 20190192077; 20190189254; 20190189259; 20190188296; U.S. Pat. No. 10,325,596; 20190179286; 20190179861; 20190175079; 20190175080; 20190167118; 20190166428; 20190159680; U.S. Pat. No. 10,299,717; 20190143990; 20190142349; 20190147430; 20190138096; 20190139050; 20190139428; 20190125264; 20190130788; 20190115107; 20190102779; 20190099118; 20190101977; 20190103182; 20190099009; 20190098395; 20190073009; 20190069848; 20190060733; U.S. Pat. No. 10,216,981; 20190053754; 20190056707; 20190046123; 20190049957; 20190050732; 20190043339; 20190038180; 20190036545; 20190037173; 20190030230; 20190022347; 20190025953; 20190008396; 20190008397; 20190011978; 20190012856; 20190000332; 20180373793; 20180373717; 20180376205; 20180376204; 20180364810; 20180360326; 20180359112; U.S. Pat. No. 10,151,636; 20180345078; 20180349416; 20180349371; 20180349386; U.S. Pat. Nos. 10,136,852; 10,130,308; 10,130,299; 10,130,311; 10,130,261; 20180326818; 20180321700; 20180317794; 20180318529; 20180310877; 20180307801; 20180303381; 20180307314; 20180308569; U.S. Pat. No. 10,105,108; 20180300664; 20180299956; 20180297586; 20180289285; U.S. Pat. No. 10,092,232; 20180279918; 20180280721; 20180279881; 20180279946; U.S. Pat. No. 10,085,685; 20180271441; U.S. Pat. No. 10,080,861; 20180263501; 20180264320; 20180264347; U.S. Pat. Nos. 10,076,270; 10,076,250; 20180260247; 20180262490; 20180256094; 20180256095; 20180254097; 20180255110; U.S. Pat. No. 10,064,559; 20180242850; 20180247713; 20180247043; 20180247067; 20180235507; 20180235542; 20180232934; U.S. Pat. Nos. 10,045,699; 10,045,726; 10,045,737; 20180220923; 20180225437; 20180220972; 20180206455; 20180206725; 20180212449; U.S. Pat. No. 10,022,614; 20180196432; 20180189398; 20180184964; 20180191888; 20180177436; 20180175905; 20180173230; 20180158542; 20180153477; 20180156660; U.S. Pat. No. 9,988,055; 20180150599; 20180150126; 20180146870; U.S. Pat. No. 9,968,264; 20180117446; 20180117447; 20180116528; 20180116578; 20180116906; 20180110939; 20180106897; 20180103901; 20180096547; 20180093121; 20180082501; 20180078843; 20180068062; 20180064394; 20180056025; 20180060757; 20180049652; 20180050171; 20180042513; 20180035938; 20180035943; U.S. Pat. No. 9,888,371; 20180033330; 20180032701; U.S. Pat. No. 9,881,503; 20180025124; 20180025143; 20180020927; 20180011973; 20180012009; 20180000426; 20180004211; 20180001184; 20180000415; 20170367614; 20170367635; 20170369069; 20170367651; 20170364650; 20170355377; 20170352267; 20170349027; 20170351990; 20170349184; 20170343375; 20170337438; 20170330044; 20170327082; 20170329329; 20170326331; 20170323639; 20170319381; 20170312614; 20170318360; 20170317705; U.S. Pat. No. 9,801,553; 20170296088; 20170301037; 20170300512; 20170293356; 20170291066; 20170281081; 20170286748; 20170273612; 20170273620; 20170274249; 20170277166; 20170264987; 20170249442; 20170249433; 20170245377; 20170235918; 20170232300; U.S. Pat. No. 9,725,036; 20170215745; 20170217445; 20170209053; 20170209055; 20170202514; U.S. Pat. No. 9,711,060; 20170196514; 20170192995; 20170188947; 20170188864; 20170188876; 20170186331; 20170169379; 20170164878; 20170164856; 20170162072; 20170163750; 20170157343; 20170142589; 20170135633; 20170127727; 20170119318; 20170105668; 20170109474; 20170110022; 20170095732; 20170098166; 20170095199; 20170095192; U.S. Pat. No. 9,610,476; 20170080255; 20170080346; 20170083927; 20170083312; U.S. Pat. No. 9,597,567; 20170078785; 20170071524; 20170076521; 20170068507; 20170063107; 20170053542; 20170042485; U.S. Pat. No. 9,560,987; 20170020399; 20170020454; 20170011210; 20170004685; 20160381534; 20160374588; 20160374567; 20160374577; 20160367187; 20160371765; 20160367138; 20160367179; 20160367196; 20160360970; 20160363914; 20160354027; 20160358155; 20160349936; 20160349790; 20160338646; 20160345060; 20160338644; 20160324432; 20160317089; 20160302393; 20160300471; 20160292365; 20160287142; 20160293042; 20160287097; 20160287073; 20160292981; 20160292984; 20160287185; 20160287937; 20160292576; 20160287098; 20160292986; 20160292988; 20160293026; 20160278706; 20160285938; 20160285492; 20160274726; 20160270727; 20160269540; 20160259977; 20160249842; 20160246936; 20160235359; 20160231830; 20160220198; 20160213314; 20160202755; 20160203663; 20160198996; 20160199685; 20160204937; 20160196726; 20160196635; 20160192039; 20160183795; 20160187654; 20160191893; 20160174853; 20160175589; 20160180068; 20160180078; 20160180222; 20160167672; 20160170996; 20160170998; 20160159632; 20160150982; 20160150978; 20160144278; 20160142478; 20160140320; 20160142407; 20160128638; 20160128604; 20160128632; U.S. Pat. No. 9,329,767; 20160107029; 20160101098; 20160091877; 20160089569; 20160091922; 20160093231; 20160084869; 20160077547; 20160066812; 20160066852; 20160066853; 20160066859; 20160070339; 20160070393; 20160072756; 20160066858; 20160061613; 20160058366; 20160058376; 20160065491; 20160065655; 20160058375; 20160058337; 20160062630; 20160061588; 20160063205; 20160055295; 20160046294; 20160045135; 20160039424; 20160042648; 20160034887; 20160029959; 20160022167; 20160021425; 20160015289; 20160015307; 20160012270; 20160008632; 20160012656; 20160012749; 20160001034; 20160004862; 20150374538; 20150373236; 20150367122; 20150366504; 20150370320; 20150365575; 20150359485; 20150359491; 20150351681; 20150351493; 20150342542; 20150348162; 20150348538; 20150350413; 20150341717; 20150335877; 20150335876; 20150332532; 20150328985; 20150332603; 20150320588; 20150324568; 20150313542; 20150314681; 20150306136; 20150305677; 20150309316; 20150309563; 20150297109; 20150297145; 20150282768; 20150282769; 20150286277; 20150278498; 20150264028; 20150262501; 20150264459; U.S. Pat. No. 9,135,803; 20150251074; 20150254955; 20150230719; 20150234187; 20150234188; 20150234206; 20150237460; 20150237461; 20150229749; 20150216475; 20150215443; 20150190669; 20150182160; 20150182130; 20150186609; 20150178915; 20150173631; 20150146355; 20150148619; 20150137994; 20150142082; 20150135310; 20150130613; 20150135284; 20150135309; 20150119725; 20150119654; 20150118967; 20150072326; 20150057512; 20150052089; 20150031964; 20140357962; 20140358012; 20140347176; 20140347265; 20140340997; 20140334653; 20140327515; 20140324749; 20140316230; 20140316305; 20140307878; 20140308930; 20140285326; 20140288435; 20140277739; 20140266939; 20140273858; 20140275850; 20140275852; 20140276119; 20140278220; 20140278229; 20140283111; 20140245785; 20140245791; 20140246497; 20140246498; 20140246499; 20140246500; 20140246501; 20140246917; 20140247136; 20140247144; 20140247146; 20140247147; 20140247149; 20140247150; 20140247151; 20140247154; 20140247155; 20140249379; 20140249760; 20140249853; 20140247137; 20140245783; 20140245784; 20140245790; 20140232534; 20140229644; 20140210640; 20140206289; U.S. Pat. No. 8,775,975; 20140188770; 20140182170; 20140168399; 20140172467; 20140160250; 20140164056; 20140165185; 20140154647; 20140156084; 20140145784; U.S. Pat. No. 8,738,925; 20140142403; 20140135612; 20140135631; 20140121982; 20140107493; 20140099623; 20140085050; 20140089672; 20140089673; 20140078049; 20140063054; 20140063055; 20140057232; 20140051961; 20140039839; 20140039840; 20140039841; 20140039842; 20140028714; 20140009262; 20130325394; 20130325396; 20130325399; 20130325404; 20130314243; 20130317382; 20130317384; 20130318546; 20130314303; 20130310907; 20130304165; 20130278631; 20130280682; 20130216989; U.S. Pat. No. 8,512,240; 20130212168; 20130203019; 20130194066; 20130190556; U.S. Pat. No. 8,494,507; 20130183646; 20130178289; 20130172693; 20130166332; 20130144537; 20130127980; 20130131465; U.S. Pat. No. 8,441,356; 20130060602; 20130044055; 20130013667; 20130002903; 20120330173; 20120316661; 20120317024; 20120313746; 20120308484; 20120295589; 20120284112; 20120284332; 20120271121; 20120258800; 20120260346; 20120249797; 20120251989; 20120245439; 20120242678; 20120242697; 20120242698; 20120235883; 20120235884; 20120235885; 20120235886; 20120235887; 20120235900; 20120236030; 20120236031; 20120229270; 20120218172; 20120218301; 20120214143; 20120212398; 20120212399; 20120212400; 20120212406; 20120212414; 20120212484; 20120212499; 20120206322; 20120206323; 20120206334; 20120206335; 20120206485; 20120200488; 20120200499; 20120200601; 20120194418; 20120194419; 20120194420; 20120194549; 20120194550; 20120194551; 20120194552; 20120194553; U.S. Pat. No. 8,196,168; 20120130800; 20120123232; 20120117094; 20120083668; 20120084054; 20120084139; 20120075168; 20120071743; 20120072289; 20120010474; 20110260830; 20110245633; 20110225536; 20110214082; 20110213664; 20110181394; 20110169603; 20110145041; 20110130796; 20110054564; 20110022332; 20100313048; 20100302000; 20100285440; 20100013739; 20090318773; 20090270170; 20090196852; 20090192961; 20090172540; 20090171164; 20090163777; 20090164131; 20090164302; 20090164401; 20090164403; 20090164458; 20090164503; 20090164549; 20090164132; 20090156907; 20090157482; 20090157660; 20090157751; 20090157813; 20090157625; 20090156955; 20090157481; 20090115617; 20090043920; 20090037634; 20090024050; 20090016645; 20090018407; 20090009284; 20080287821; 20080208016; 20080085055; 20080080700; 20070299322; 20070293781; 20070263092; 20070263978; 20070242138; 20070242945; 20070100666; 20070063850; 20070059711; 20070038035; 20060293921; 20060252761; 20060084659; U.S. Pat. No. 6,980,931; 20050266438; 20050240956; 20050113703; 20050096396; 20050088297; 20040229685; 20040208923; 20040101212; 20040101178; 20030165269; 20030165270; 20030156304; 20030128389; 20030122839; 20030117651; U.S. Pat. Nos. 6,331,534; 6,132,724; 6,117,860; 6,066,627; and 6,057,439.

In another embodiment, the measuring device comprises position sensors adapted to track the body position of the subject. Body position is a critical factor in sleep quality, as certain positions can either alleviate or exacerbate conditions such as sleep apnea or snoring.

These position sensors can be integrated into a wearable device, such as a belt or attached to the subject's sleepwear or embedded within the mattress. They provide data on the subject's body orientation and movements. This information is transmitted to the control unit, which can adjust the auditory stimuli based on the detected body position. For example, if the subject is detected to be in a position that typically causes discomfort or sleep disturbances, the control unit can provide stimuli to encourage a position change.

In another embodiment, the measuring device comprises non-invasive blood pressure sensors adapted to monitor the blood pressure of the subject. Blood pressure is a vital indicator of cardiovascular health and can fluctuate significantly during different sleep stages.

The blood pressure sensor can for example be worn as a cuff on the wrist or upper arm and are designed to be comfortable for use throughout the night. The sensor provides data on the subject's blood pressure levels, which is transmitted to the control unit. The control unit then adjusts the auditory stimuli as needed. For example, if elevated blood pressure is detected, the control unit can deliver calming stimuli to help lower the blood pressure and promote a more restful sleep state.

In another embodiment, the measuring device comprises sensors adapted to measure peripheral blood flow using plethysmography. This method involves detecting blood volume changes in the peripheral circulation, which can provide insights into the subject's cardiovascular health and sleep stages.

These plethysmography sensors can be integrated into a wearable device, such as a finger clip or wristband, that the subject wears during sleep. The sensors provide continuous data on peripheral blood flow, which is transmitted to the control unit. The control unit uses this information to adjust the auditory stimuli to optimize sleep conditions. For instance, if the sensors detect reduced peripheral blood flow, the control unit can modify the stimuli to encourage better circulation and deeper sleep.

In another embodiment, the control unit comprises a standard or individualized Head-Related Transfer Function (HRTF) to improve the perception of spatial sounds (spatialized audio). This embodiment enhances the device's capability to create a realistic and immersive auditory environment, which helps for inducing a sensation of motion and promoting relaxation.

The HRTF is a response that characterizes how an ear receives a sound from a point in space. By using HRTFs, the control unit can simulate the way sound waves interact with the subject's head and ears, creating the perception that sounds are coming from specific directions and distances. This technique is helpful for accurately reproducing spatial audio cues.

In this embodiment, the control unit can use either a standard HRTF or an individualized HRTF. A standard HRTF is based on average measurements from a large population, providing a general spatial audio experience that works well for most users. However, for a more personalized and precise auditory experience, an individualized HRTF can be used. This individualized HRTF is generated based on specific measurements of the subject's ear shape and head geometry, which can be obtained through advanced scanning techniques or personalized calibration processes.

The control unit uses the HRTF to process the auditory stimuli and position sounds accurately around the subject. For example, sounds can be made to appear as if they are coming from above, behind, or from the sides, creating a three-dimensional audio environment. This spatial audio capability is particularly effective in enhancing the sensation of motion, such as rocking or swaying, and can be used to simulate environments like a moving vehicle or natural surroundings.

The implementation of HRTF allows for instance, during light sleep or periods of restlessness, to use spatial sounds that gently move around the subject, promoting a calming effect. During deeper sleep stages, the spatial audio can be minimized or positioned in a way that maintains the subject in a stable and restful state without causing disruptions.

See, U.S. Patent and Pub. Pat. Application Nos. 20240231093; 20240233099; 20240233100; 20240236256; 20240236605; 20240236613; 20240236596; 20240236602; 20240236603; 20240236535; 20240236595; 20240233702; 20240236611; 20240236601; U.S. Pat. Nos. 12,035,117; 12,035,130; 12,035,126; 12,035,124; 12,035,129; 20240221337; 20240223990; 20240223625; 20240219562; 20240220738; 20240214765; 20240211563; 20240214728; 20240214709; 20240214759; 20240205634; 20240202944; 20240203055; 20240201456; 20240198578; 20240205632; 20240204549; 20240196151; 20240196159; 20240192481; 20240196156; 20240194215; U.S. Pat. Nos. 12,009,877; 12,010,493; 12,010,494; 20240184361; 20240181201; 20240187807; 20240187806; 20240187790; 20240187809; 20240179487; U.S. Pat. Nos. 11,997,454; 11,997,463; 20240171927; 20240168300; 20240171682; U.S. Pat. No. 11,989,404; 20240163529; 20240163624; 20240163630; 20240155283; 20240151882; 20240152817; U.S. Pat. No. 11,979,667; 20240147181; 20240147177; 20240140468; 20240137724; 20240135912; 20240137728; 20240137723; 20240137720; 20240137677; 20240129681; 20240129685; 20240126371; 20240126990; 20240121570; 20240119945; 20240119946; 20240118423; 20240118746; 20240114309; 20240114313; 20240114312; 20240103613; 20240105196; 20240107259; 20240104871; 20240104872; 20240103617; 20240103705; 20240098428; 20240098447; 20240094390; 20240098444; 20240098442; 20240098404; 20240098439; 20240096340; 20240096334; U.S. Pat. No. 11,937,073; 20240089692; 20240089690; 20240089683; 20240089689; 20240087589; 20240089687; 20240080633; 20240078995; 20240080618; 20240080638; 20240078743; U.S. Pat. Nos. 11,924,628; 11,924,317; 20240071394; 20240071411; 20240073564; 20240071350; 20240073589; 20240064446; 20240059201; 20240061249; 20240062403; 20240064486; 20240056760; 20240051447; 20240056762; 20240056756; 20240054158; 20240056745; 20240048935; 20240048934; 20240045216; 20240045942; 20240045035; 20240037886; U.S. Pat. No. 11,885,147; 20240031766; 20240031758; 20240031759; 20240031763; 20240031767; 20240031765; 20240031744; 20240031742; 20240021186; 20240022855; U.S. Pat. No. 11,875,813; 20240015463; 20240015460; 20240012609; 20240015461; 20240015466; 20240007819; 20240005622; 20230419625; 20230419985; 20230421981; 20230418383; 20230418076; 20230418022; 20230421945; U.S. Pat. No. 11,852,843; 20230413005; 20230413000; 20230412980; 20230413003; 20230410348; 20230403528; 20230403526; 20230403443; 20230403529; 20230401805; 20230396950; 20230396920; 20230394886; 20230388690; 20230386461; 20230388732; 20230388731; 20230387729; 20230384860; 20230380739; 20230377276; 20230377194; 20230379646; 20230379645; 20230364513; 20230370801; 20230370804; 20230370797; 20230368801; 20230370765; 20230370800; 20230368475; U.S. Pat. No. 11,815,692; 20230362528; 20230362537; 20230362545; 20230362571; 20230362579; U.S. Pat. Nos. 11,809,774; 11,812,194; 20230353988; 20230353678; 20230353930; 20230345195; 20230345168; U.S. Pat. No. 11,796,823; 20230336912; 20230336926; 20230336938; 20230334799; 20230329913; 20230328461; 20230320669; 20230319469; 20230319190; 20230319474; 20230315815; U.S. Pat. No. 11,778,361; 20230306953; 20230308821; 20230305306; 20230306695; 20230308630; 20230308605; 20230300532; 20230300557; 20230300556; 20230293998; 20230298226; 20230289993; 20230290270; U.S. Pat. Nos. 11,756,525; 11,758,348; 20230282965; 20230282962; 20230283976; 20230282177; U.S. Pat. No. 11,751,003; 20230276191; 20230276186; 20230276189; 20230276187; U.S. Pat. No. 11,741,898; 20230269553; 20230262406; 20230262405; 20230257362; 20230260222; 20230262169; 20230260268; 20230254661; 20230254656; 20230254660; 20230254637; U.S. Pat. No. 11,722,137; 20230247381; 20230247384; 20230247380; U.S. Pat. No. 11,715,479; 20230236034; 20230239626; 20230225905; 20230232178; 20230222687; 20230218998; 20230224659; 20230224663; 20230224667; 20230224668; 20230217206; 20230217201; 20230216269; 20230209302; 20230209298; 20230209299; 20230208921; 20230209266; 20230209294; 20230199417; 20230199421; 20230199418; 20230196765; 20230190173; 20230194869; U.S. Pat. No. 11,683,634; 20230188893; 20230186781; U.S. Pat. No. 11,678,111; 20230179946; 20230179941; 20230179947; 20230179945; 20230171553; 20230171542; 20230171556; U.S. Pat. Nos. 11,659,043; 11,659,324; 20230156404; 20230156403; 20230152583; 20230143325; 20230143857; 20230146178; 20230147816; 20230136085; 20230132745; 20230134271; 20230134249; 20230139061; 20230139295; 20230140699; 20230132774; 20230137514; 20230130930; 20230130524; 20230118803; 20230116044; 20230110257; 20230114777; 20230113703; 20230106761; 20230109140; 20230104111; 20230096873; 20230099728; 20230096953; 20230096119; 20230099463; 20230099275; 20230098577; 20230088922; 20230089225; 20230093585; 20230090315; 20230091218; 20230087729; 20230079683; 20230079940; 20230081104; 20230083937; 20230077689; 20230080909; 20230085063; 20230070037; 20230072423; U.S. Pat. No. 11,598,962; 20230068347; 20230065296; 20230067081; 20230068527; U.S. Pat. No. 11,595,754; 20230055257; 20230054213; U.S. Pat. Nos. 11,589,181; 11,589,184; 11,589,176; 20230046511; 20230049597; 20230051841; 20230050966; 20230039360; 20230040846; 20230041730; 20230038945; 20230044474; 20230045721; 20230042762; 20230041038; 20230041678; 20230045207; 20230045569; 20230045610; 20230044624; 20230040821; 20230037463; U.S. Pat. Nos. 11,576,005; 11,568,562; 11,570,538; 20230027060; 20230025801; U.S. Pat. Nos. 11,563,855; 11,564,038; 20230019204; 20230015418; 20230015732; 20230017111; 20230020792; 20230015199; 20230011591; 20230011408; 20230008865; 20230008964; 20230011087; 20230012320; 20230007420; 20230007435; 20230007430; 20230007424; 20230007432; 20230007421; 20230005464; 20230000684; U.S. Pat. No. 11,546,687; 20220417691; 20220417694; 20220413296; U.S. Pat. Nos. 11,537,204; 11,540,055; 20220405043; 20220405946; 20220400352; 20220400330; 20220394414; 20220394407; 20220394406; 20220391899; U.S. Pat. Nos. 11,523,244; 11,523,242; 20220383885; 20220380372; 20220386060; 20220383881; 20220386062; 20220385748; 20220385796; U.S. Pat. No. 11,513,578; 20220377489; 20220377485; 20220377486; 20220377491; 20220371527; 20220377461; 20220373803; U.S. Pat. No. 11,510,004; 20220369058; 20220369061; 20220366919; 20220366723; 20220369035; 20220368554; 20220369034; 20220366933; 20220360899; 20220360931; 20220360930; 20220360891; 20220360934; U.S. Pat. No. 11,490,218; 20220345813; 20220345820; 20220345845; 20220345787; 20220343289; 20220345844; 20220342213; U.S. Pat. No. 11,481,033; 20220332700; 20220337969; 20220337952; 20220335923; 20220337651; 20220337910; 20220328056; 20220323427; 20220327233; 20220329957; 20220329961; 20220329959; 20220327225; U.S. Pat. No. 11,470,439; 20220322001; 20220321992; 20220322021; 20220322010; 20220322019; 20220322009; 20220322023; 20220322024; 20220321999; 20220322028; 20220312143; 20220312144; U.S. Pat. No. 11,454,700; 20220303710; 20220303690; 20220303657; 20220295212; 20220295213; 20220294980; 20220294100; 20220295581; 20220291743; 20220291513; 20220295287; U.S. Pat. No. 11,445,290; 20220286781; 20220286751; 20220286788; 20220286798; U.S. Pat. Nos. 11,436,987; 11,435,528; 20220279300; 20220279303; 20220279304; U.S. Pat. No. 11,432,095; 20220272481; 20220272477; 20220272454; 20220272449; 20220270626; U.S. Pat. No. 11,425,515; 20220264242; 20220264223; 20220254355; 20220256300; 20220256269; 20220256287; U.S. Pat. No. 11,409,360; 20220243202; 20220248161; 20220246128; 20220240043; 20220232342; U.S. Pat. No. 11,395,087; 20220225049; 20220225016; 20220225050; U.S. Pat. No. 11,388,513; 20220202190; 20220208086; 20220208134; 20220210596; 20220206566; U.S. Pat. No. 11,375,333; 20220192541; 20220194909; 20220201418; 20220201388; 20220201419; 20220201403; 20220191627; 20220191638; 20220187794; 20220187883; 20220191578; 20220187655; 20220187926; 20220175784; 20220182778; 20220182777; 20220182772; 20220180885; 20220174447; 20220174446; 20220174428; 20220174448; 20220167109; 20220165281; 20220167111; 20220167105; 20220167097; U.S. Pat. No. 11,340,463; 20220159402; 20220155778; 20220159396; 20220150657; 20220148115; 20220150653; 20220150658; 20220137916; 20220141581; 20220141604; 20220141599; 20220141611; 20220141608; 20220135974; 20220141607; 20220139405; 20220125342; 20220126837; 20220129062; U.S. Pat. No. 11,315,277; 20220124448; 20220124446; 20220124447; 20220116724; 20220116725; 20220115007; U.S. Pat. No. 11,303,258; 20220101126; 20220103962; 20220103948; 20220101734; U.S. Pat. Nos. 11,288,767; 11,287,885; 11,290,802; 11,290,837; 20220095073; 20220095051; 20220095071; 20220086583; 20220086589; 20220082688; 20220086585; 20220079813; 20220078338; 20220078571; 20220078572; 20220078374; 20220066542; 20220061767; U.S. Pat. Nos. 11,262,807; 11,265,668; 20220060824; 20220060841; 20220060820; U.S. Pat. No. 11,259,139; 20220053282; 20220053281; 20220049944; U.S. Pat. No. 11,250,833; 20220042646; U.S. Pat. No. 11,246,002; 20220038838; 20220038832; 20220036625; 20220023137; 20220030375; 20220027033; 20220026986; 20220028407; 20220030369; 20220030377; U.S. Pat. Nos. 11,234,078; 11,234,095; 11,234,073; 20220021975; 20220021976; 20220021996; 20220021973; 20220021972; U.S. Pat. No. 11,226,406; 20220014865; 20220013991; 20220014868; 20220014712; 20220014680; U.S. Pat. No. 11,222,549; 20220007078; 20220005470; 20220007112; 20220007127; 20210409879; 20210409876; 20210407244; 20210409888; 20210405959; U.S. Pat. No. 11,212,606; 20210400417; 20210400419; 20210397407; 20210398545; 20210392448; 20210392223; 20210383820; 20210385600; 20210385605; 20210377685; 20210377688; 20210377659; 20210377643; 20210377684; 20210377662; 20210373654; 20210375257; U.S. Pat. No. 11,190,896; 20210368285; 20210366142; 20210358506; 20210358507; 20210354137; U.S. Pat. No. 11,176,925; 20210352425; 20210352408; 20210350604; U.S. Pat. No. 11,172,320; 20210345057; 20210337339; 20210337338; 20210337340; 20210333402; 20210337336; 20210337341; U.S. Pat. No. 11,159,768; 20210329403; 20210329396; 20210329399; 20210329381; 20210322880; 20210325683; 20210321212; 20210314721; 20210314710; 20210311553; 20210306786; 20210301581; 20210306751; 20210292328; 20210297780; 20210297781; 20210297798; 20210297805; 20210297802; 20210297806; 20210297803; U.S. Pat. No. 11,125,561; 20210289297; 20210289309; 20210289306; 20210287651; 20210281964; 20210281966; 20210281232; U.S. Pat. Nos. 11,112,389; 11,115,773; 20210272574; 20210274296; 20210274283; 20210264931; 20210266693; 20210264927; U.S. Pat. Nos. 11,098,737; 11,102,606; 11,102,578; 11,102,602; 20210258711; 20210258690; 20210258670; 20210256261; 20210256769; 20210256175; 20210258715; 20210258713; U.S. Pat. Nos. 11,096,004; 11,096,006; 20210250686; 20210250723; 20210248827; 20210250682; 20210250701; U.S. Pat. No. 11,087,777; 20210235189; 20210231488; 20210234611; U.S. Pat. No. 11,074,036; 20210227150; 20210227320; U.S. Pat. No. 11,070,933; 20210219084; 20210219065; 20210219071; 20210219089; 20210211806; 20210211207; 20210211828; 20210211826; 20210211825; 20210211827; 20210211821; 20210211823; 20210204084; 20210204058; 20210204085; 20210195358; 20210195327; 20210195361; 20210195360; 20210195336; 20210194942; U.S. Pat. No. 11,039,651; 20210185174; 20210185175; 20210185470; 20210185471; U.S. Pat. Nos. 11,039,265; 11,039,266; 20210176579; 20210176577; 20210176565; 20210176583; 20210176584; 20210176580; 20210176588; 20210168548; 20210168549; 20210168518; 20210168551; 20210168554; 20210160642; 20210160605; 20210160474; 20210160645; 20210156986; 20210160644; U.S. Pat. No. 11,012,804; 20210144504; 20210144475; 20210144506; U.S. Pat. No. 11,006,197; 20210134314; 20210136507; 20210136509; 20210136508; U.S. Pat. No. 10,998,006; 20210127223; 20210127220; 20210125412; 20210118218; 20210112232; 20210112360; U.S. Pat. No. 10,976,543; 20210103732; 20210105565; 20210105552; 20210105435; U.S. Pat. Nos. 10,972,834; 10,971,130; 20210099795; 20210099826; 20210099825; U.S. Pat. No. 10,966,043; 20210092545; 20210092544; 20210089260; 20210090546; 20210084414; 20210082191; 20210084429; 20210084357; 20210084411; 20210084424; 20210081048; U.S. Pat. Nos. 10,948,976; 10,948,989; 20210076153; 20210076130; 20210076150; 20210075074; U.S. Pat. No. 10,945,090; 20210067896; 20210067891; 20210065523; 20210058693; 20210056984; U.S. Pat. Nos. 10,932,081; 10,928,889; 20210051435; 20210050023; 20210047287; 20210040087; 20210043185; 20210043223; 20210043005; 20210042994; 20210037336; 20210037333; 20210034725; 20210030308; 20210035597; 20210037335; U.S. Pat. Nos. 10,907,371; 10,911,885; 20210029486; 20210029472; 20210029479; 20210029480; 20210018102; U.S. Pat. No. 10,897,570; 20210014631; 20210012782; 20210014457; 20210014614; 20210014597; 20210014596; U.S. Pat. Nos. 10,891,890; 10,893,357; 20210006929; 20210006976; 20210004452; 20210004201; 20210004200; 20210005213; 20210006922; 20210006918; 20210006923; 20210006921; 20210006925; 20200413211; 20200413190; 20200409481; U.S. Pat. Nos. 10,880,667; 10,880,668; 20200404214; 20200404422; 20200402523; 20200402529; 20200402519; 20200404441; 20200402521; 20200402522; 20200404446; U.S. Pat. No. 10,873,800; 20200394288; 20200389755; 20200388291; 20200389751; 20200385012; 20200387341; 20200388065; 20200382894; 20200382895; 20200382871; U.S. Pat. No. 10,856,082; 20200368616; 20200371737; 20200374646; 20200374647; 20200372919; 20200374645; U.S. Pat. No. 10,848,859; 20200366990; 20200367005; U.S. Pat. No. 10,841,728; 20200359130; 20200356341; 20200351606; 20200345757; U.S. Pat. Nos. 10,823,960; 10,824,390; 10,824,247; 20200344563; 20200342846; U.S. Pat. No. 10,819,953; 20200336858; 20200336856; 20200335128; U.S. Pat. Nos. 10,812,887; 10,812,928; 10,812,929; 20200329327; 20200329330; 20200329317; 20200327877; 20200329088; 20200326199; U.S. Pat. No. 10,805,740; 20200322727; 20200319842; 20200322735; 20200321000; 20200320999; 20200319634; U.S. Pat. No. 10,798,511; 20200312347; 20200314562; 20200312025; 20200311951; 20200314581; 20200307620; 20200314524; U.S. Pat. Nos. 10,791,389; 10,788,897; 20200301562; 20200301653; 20200304935; 20200296500; 20200296533; 20200293268; 20200294502; 20200288262; 20200286450; 20200280816; 20200280815; 20200279561; U.S. Pat. Nos. 10,764,707; 10,764,684; 20200275195; 20200275232; 20200275216; U.S. Pat. Nos. 10,754,428; 10,757,528; 10,757,240; 20200267492; 20200267486; 20200267487; 20200260206; 20200260209; 20200260199; 20200257548; 20200258306; U.S. Pat. No. 10,743,128; 20200252739; 20200245869; U.S. Pat. No. 10,735,885; 20200245092; 20200245091; U.S. Pat. Nos. 10,728,690; 10,728,684; 20200236488; U.S. Pat. No. 10,721,521; 20200227052; 20200228913; 20200228915; 20200228911; U.S. Pat. Nos. 10,715,933; 10,715,909; 20200221230; 20200221241; 20200221240; 20200221243; 20200218767; U.S. Pat. Nos. 10,708,706; 10,706,693; 20200209952; 20200207737; 20200210051; 20200211574; 20200213802; 20200213738; 20200213702; 20200213711; 20200210127; U.S. Pat. No. 10,701,426; 20200202837; 20200204966; 20200204940; 20200204280; 20200201495; 20200196058; 20200196085; 20200186912; 20200186955; 20200179389; 20200186953; 20200186917; 20200186954; 20200175997; 20200178018; 20200177994; 20200178016; 20200168235; 20200169827; U.S. Pat. No. 10,667,073; 20200160600; 20200162833; 20200162817; 20200162140; 20200158865; U.S. Pat. No. 10,659,875; 20200154231; 20200150751; 20200154229; 20200152203; 20200152138; U.S. Pat. Nos. 10,650,798; 10,649,060; 20200145778; 20200145776; 20200142667; 20200145755; 20200142664; 20200142665; U.S. Pat. No. 10,645,520; 20200137508; 20200137488; 20200137509; 20200137489; 20200137494; 20200134026; 20200135163; U.S. Pat. Nos. 10,638,248; 10,638,222; 10,636,260; 10,638,252; 20200128348; 20200125317; 20200128233; 20200114834; 20200112812; 20200112814; 20200112817; 20200112815; 20200111262; 20200111489; 20200108720; 20200107147; 20200107148; 20200105282; 20200104620; 20200107149; 20200103964; 20200094141; 20200100044; 20200097248; 20200099792; U.S. Pat. Nos. 10,602,300; 10,602,302; 20200089466; 20200087318; U.S. Pat. No. 10,595,149; 20200084538; 20200084560; 20200084565; 20200084563; 20200077223; 20200077222; 20200077221; 20200068309; 20200068335; 20200068334; 20200068337; 20200066047; U.S. Pat. Nos. 10,575,094; 10,574,472; 20200057493; 20200059724; 20200059750; 20200058310; 20200058311; 20200051325; 20200053503; 20200053502; 20200042792; 20200045419; 20200045490; 20200045493; 20200045491; U.S. Pat. No. 10,555,106; 20200037091; 20200037098; 20200037097; 20200035259; 20200029155; 20200026922; 20200029164; U.S. Pat. No. 10,542,368; 20200021927; 20200021938; 20200021939; 20200010453; 20200015014; 20200015007; 20200013426; 20200013414; U.S. Pat. No. 10,531,219; 20200008004; 20200005608; U.S. Pat. Nos. 10,524,080; 10,522,008; 20190394580; 20190394607; 20190394583; 20190394564; 20190391783; 20190392845; 20190392846; 20190394568; 20190394569; 20190394598; 20190394602; 20190394603; 20190394601; 20190394565; 20190394567; 20190394570; U.S. Pat. No. 10,516,959; 20190387354; 20190385587; 20190387346; 20190387352; 20190387324; 20190387340; U.S. Pat. Nos. 10,511,909; 10,509,466; 10,511,906; 20190379975; 20190379992; 20190379997; 20190376755; 20190378052; 20190378385; 20190373398; 20190373400; 20190373368; 20190373385; 20190373357; 20190373366; 20190373391; 20190373395; U.S. Pat. Nos. 10,499,180; 10,499,179; 20190364352; U.S. Pat. Nos. 10,491,981; 10,492,018; 20190356999; 20190355172; U.S. Pat. No. 10,484,809; 20190349681; 20190349703; 20190349702; U.S. Pat. Nos. 10,477,157; 10,477,338; 20190342665; 20190341060; 20190335292; 20190335287; 20190329364; 20190333397; 20190335288; U.S. Pat. No. 10,462,598; 20190327575; 20190327571; 20190318510; 20190320114; 20190313174; 20190308114; 20190313198; 20190313201; 20190313200; U.S. Pat. Nos. 10,440,498; 10,440,473; 20190306650; 20190304081; 20190306604; 20190297447; U.S. Pat. No. 10,425,762; 20190289420; 20190284293; 20190289417; 20190289414; U.S. Pat. Nos. 10,419,870; 10,419,843; 20190278802; 20190278552; U.S. Pat. No. 10,412,527; 20190273990; 20190270118; 20190268697; U.S. Pat. Nos. 10,397,727; 10,397,725; 20190261124; 20190260930; 20190261095; U.S. Pat. No. 10,390,170; 20190253821; 20190251955; 20190246230; 20190246231; 20190244326; U.S. Pat. No. 10,375,506; 20190238980; 20190230430; 20190218202; 20190215640; 20190215637; 20190213792; 20190212824; 20190215632; 20190208348; 20190208349; 20190201658; 20190200097; 20190200159; 20190187954; 20190191264; 20190191254; 20190191248; 20190187779; 20190191247; 20190182587; 20190182612; 20190180509; 20190182613; 20190182594; 20190182592; 20190180731; 20190174246; 20190174237; 20190174248; 20190170533; 20190166448; 20190166419; 20190160083; U.S. Pat. No. 10,306,395; 20190155404; 20190147650; 20190149939; 20190139312; 20190138207; 20190139554; 20190132695; 20190132674; 20190130927; 20190132685; 20190132680; 20190130928; 20190124463; 20190124447; 20190122681; 20190116440; 20190116445; 20190116448; 20190116451; 20190116452; 20190116447; 20190110137; 20190110152; 20190110125; 20190110148; 20190104366; U.S. Pat. No. 10,251,014; 20190095451; 20190098426; 20190098427; 20190090571; 20190090060; 20190087972; U.S. Pat. Nos. 10,237,675; 10,231,053; 20190075418; 20190075417; U.S. Pat. Nos. 10,225,682; 10,225,681; 20190066701; 20190062442; 20190069118; 20190069105; 20190069104; 20190069114; 20190069110; 20190060741; 20190056848; 20190057529; 20190057612; U.S. Pat. No. 10,212,532; 20190052946; 20190050193; U.S. Pat. No. 10,206,053; 20190045318; 20190045320; 20190042844; 20190045317; 20190035410; 20190037331; 20190037334; 20190030277; 20190025416; 20190028803; 20190019520; 20190020963; 20190019495; 20190020968; 20190018641; 20190014433; 20190012835; 20190014431; 20190007783; 20190007784; 20190007725; 20180369689; 20180373957; 20180374276; U.S. Pat. Nos. 10,165,386; 10,163,271; 20180367937; 20180366136; 20180367935; 20180359596; 20180359594; 20180359294; U.S. Pat. No. 10,154,364; 20180352363; 20180352334; 20180352070; U.S. Pat. No. 10,149,089; 20180343535; 20180343534; 20180343432; U.S. Pat. No. 10,142,760; 20180338215; 20180335903; U.S. Pat. No. 10,136,239; 20180332426; 20180332422; 20180332424; 20180332423; 20180324541; 20180324540; 20180324542; 20180324532; 20180324530; 20180317003; 20180314486; 20180315437; 20180314488; 20180315435; 20180317034; 20180315433; 20180317039; U.S. Pat. No. 10,113,877; 20180310114; 20180310098; 20180310113; 20180310116; 20180310115; U.S. Pat. No. 10,110,994; 20180300940; 20180300551; 20180300952; 20180295462; 20180295463; 20180293798; 20180288554; 20180288558; 20180284882; 20180288518; 20180279063; 20180276476; 20180275861; 20180279065; 20180270603; 20180262849; 20180262858; 20180253275; U.S. Pat. No. 10,070,224; 20180249275; 20180249279; 20180249276; 20180249269; 20180249274; 20180249271; U.S. Pat. No. 10,063,990; 20180242077; 20180242094; 20180233156; 20180232471; 20180226105; 20180225805; 20180220253; 20180218507; 20180216953; 20180220251; 20180220255; 20180218740; 20180218727; 20180220252; U.S. Pat. No. 10,038,966; 20180213341; 20180213343; U.S. Pat. No. 10,034,092; 20180206039; 20180206054; 20180206058; 20180206056; 20180204341; 20180206038; U.S. Pat. No. 10,028,070; 20180199137; 20180197551; 20180192228; 20180190005; 20180192185; U.S. Pat. No. 10,015,618; 20180184227; 20180184226; 20180184225; U.S. Pat. Nos. 10,009,704; 10,009,690; 20180168453; 20180174592; U.S. Pat. No. 10,003,905; 20180160251; U.S. Pat. Nos. 9,992,599; 9,992,532; 9,992,570; 9,992,602; 20180152787; 20180144752; U.S. Pat. No. 9,980,075; 20180139563; 20180133477; 20180139567; 20180139530; 20180139565; 20180132054; 20180132052; 20180132053; U.S. Pat. No. 9,967,693; 20180122396; 20180124543; 20180124526; 20180117469; 20180115769; 20180115854; 20180115852; U.S. Pat. Nos. 9,955,277; 9,955,281; 20180109901; 20180109900; 20180103316; 20180101990; 20180103334; U.S. Pat. Nos. 9,942,687; 9,940,922; 20180098175; 20180091920; 20180091926; 20180091919; 20180091921; 20180091917; 20180078422; 20180077514; 20180061136; 20180063641; 20180063662; 20180053413; U.S. Pat. No. 9,900,555; 20180048978; 20180048959; 20180046431; 20180048979; 20180041848; 20180041849; 20180036175; 20180035226; 20180035233; 20180034867; 20180035234; 20180035236; 20180020298; 20180020297; 20180020312; 20180016252; 20180015878; U.S. Pat. No. 9,872,121; 20180014135; 20180014130; 20180007489; 20180007485; 20180007486; 20180007488; 20180007461; U.S. Pat. No. 9,860,636; 20170374317; 20170374444; 20170366914; U.S. Pat. No. 9,848,273; 20170359671; 20170359467; 20170353811; 20170353812; 20170346951; 20170344852; 20170347192; 20170347193; 20170345216; U.S. Pat. No. 9,832,587; 20170339504; 20170339506; 20170332184; 20170332187; 20170332186; 20170330365; 20170330382; 20170325042; 20170325046; 20170324931; 20170325045; 20170325043; 20170318372; 20170318407; 20170303052; 20170295445; 20170295446; 20170295430; U.S. Pat. No. 9,786,288; 20170289729; 20170289730; U.S. Pat. No. 9,774,976; 20170272698; 20170272870; 20170272889; 20170272890; 20170272882; 20170272863; U.S. Pat. No. 9,769,585; 20170265016; 20170257697; 20170257723; 20170257724; 20170251323; 20170249714; 20170251324; U.S. Pat. No. 9,747,282; 20170243597; 20170245055; 20170245092; 20170245082; 20170240525; 20170245088; 20170238084; 20170238096; 20170238102; 20170237789; 20170236320; 20170238111; 20170230772; 20170230751; 20170228043; 20170228042; 20170223475; 20170223477; 20170223459; 20170223446; 20170223478; 20170215011; 20170215018; 20170212361; 20170208392; 20170208415; 20170208417; U.S. Pat. No. 9,711,126; 20170195766; 20170193981; 20170195793; 20170195817; U.S. Pat. No. 9,699,583; 20170188169; 20170188175; 20170188172; 20170188168; 20170180863; 20170180873; U.S. Pat. Nos. 9,674,661; 9,667,889; 20170150266; 20170134877; U.S. Pat. No. 9,648,438; 20170127211; 20170127035; 20170127210; 20170119994; 20170123492; 20170118458; 20170112671; 20170105083; 20170098452; 20170094440; 20170086005; 20170085990; 20170085996; 20170085995; U.S. Pat. No. 9,602,946; 20170078819; 20170078821; 20170078488; 20170078820; 20170078447; 20170070838; U.S. Pat. No. 9,591,427; 20170064485; 20170064444; U.S. Pat. Nos. 9,583,106; 9,584,653; 9,584,946; 20170043162; 20170048641; 20170046123; 20170045941; 20170034639; 20170032495; 20170034641; 20170026771; 20170026751; 20170019746; 20170010671; 20170013389; U.S. Pat. No. 9,544,706; 20170001966; 20170002001; 20160373877; 20160360326; 20160360334; 20160345095; 20160345116; 20160345115; 20160337779; 20160336022; 20160330559; 20160330547; 20160330560; 20160324478; 20160323688; U.S. Pat. No. 9,473,871; 20160299738; U.S. Pat. No. 9,464,912; 20160295341; 20160295539; U.S. Pat. No. 9,462,406; 20160286329; 20160286322; 20160277836; 20160277865; 20160272706; 20160266865; 20160269845; 20160269831; 20160269849; 20160269833; 20160260441; 20160255453; 20160255452; 20160249151; 20160247518; 20160241963; 20160241962; 20160241986; 20160239252; 20160241980; 20160232901; 20160234609; 20160228771; 20160225377; 20160227338; 20160227337; 20160207929; 20160212560; 20160212554; 20160212272; 20160205488; U.S. Pat. No. 9,392,366; 20160198281; 20160195856; U.S. Pat. No. 9,384,741; 20160183011; 20160174013; 20160174009; 20160165363; 20160165350; U.S. Pat. No. 9,363,569; 20160145350; 20160150339; 20160142854; 20160142851; 20160142848; 20160133267; 20160134987; 20160134988; 20160134958; 20160124707; 20160119737; 20160119734; 20160119733; 20160119731; U.S. Pat. No. 9,324,313; 20160112819; 20160109284; 20160112811; 20160104491; 20160100268; 20160094929; 20160088417; 20160088393; 20160080886; 20160080882; 20160078879; 20160077796; 20160073215; 20160073198; 20160066104; 20160066118; 20160056787; 20160057525; 20160057529; 20160049915; 20160050509; 20160044430; 20160044432; 20160044434; 20160044436; 20160036987; 20160037282; 20160037281; 20160034248; U.S. Pat. No. 9,253,566; 20160029144; 20160027447; 20160029139; 20160021480; 20160021478; 20160012816; U.S. Pat. Nos. 9,237,398; 9,230,549; 20150382127; 20150380054; 20150382129; 20150376188; 20150371644; 20150373477; 20150358754; 20150358715; 20150358755; 20150350804; 20150350801; 20150350802; 20150348530; 20150340043; 20150341738; 20150326963; 20150325230; 20150326966; 20150326973; 20150326987; 20150317981; 20150319530; 20150312694; 20150304790; 20150304758; 20150304791; 20150296086; 20150293655; 20150294041; 20150296061; 20150286459; 20150289063; 20150271620; 20150256613; 20150249898; 20150243270; 20150245157; 20150244869; 20150244868; 20150237446; 20150230040; 20150230036; 20150230026; 20150223002; 20150223005; U.S. Pat. No. 9,100,735; 20150211858; 20150208190; 20150208184; 20150201889; 20150194158; 20150189423; 20150189440; 20150189449; 20150189455; 20150189457; 20150179179; 20150180433; 20150181355; 20150172812; U.S. Pat. No. 9,053,562; 20150156578; 20150156595; 20150156599; 20150131824; 20150124167; 20150124975; 20150125010; 20150117685; 20150117659; 20150119130; 20150110275; 20150106475; 20150104022; 20150100991; U.S. Pat. No. 9,002,020; 20150092950; 20150092965; 20150088530; U.S. Pat. Nos. 8,988,970; 8,989,417; 20150080371; 20150078594; 20150078556; 20150078602; U.S. Pat. No. 8,983,834; 20150071446; 20150072974; 20150063610; 20150063553; 20150063572; 20150063577; 20150055770; 20150055783; 20150058102; 20150049872; 20150049868; 20150052455; 20150049892; 20150049004; 20150049894; U.S. Pat. No. 8,958,567; 20150039302; 20150036827; 20150031676; 20150030159; 20150030160; 20150032240; 20150023505; 20150016225; 20150017615; 20150016660; 20150010155; 20150010160; 20150003653; 20150003649; 20140379108; 20140376754; 20140376753; 20140375683; 20140372107; 20140364447; 20140364682; 20140355765; 20140355796; 20140350944; 20140348331; 20140348360; 20140341547; 20140334626; 20140330344; 20140328505; 20140330160; U.S. Pat. No. 8,879,743; 20140321660; 20140321680; 20140320586; 20140307877; U.S. Pat. No. 8,852,103; 20140297296; 20140286511; 20140270185; 20140270280; 20140270182; 20140254828; U.S. Pat. No. 8,831,231; 20140233716; 20140233917; U.S. Pat. No. 8,812,013; 20140219457; 20140219485; 20140221017; 20140211971; 20140198918; 20140193009; 20140185844; 20140185811; 20140185846; 20140185847; 20140185849; 20140177845; 20140171195; 20140166122; 20140164001; 20140153765; 20140153727; 20140133661; 20140133660; 20140133683; 20140133658; U.S. Pat. No. 8,718,301; 20140118631; 20140112482; 20140114560; 20140107916; 20140108934; 20140098963; 20140086414; 20140079212; 20140079267; 20140064527; 20140064526; U.S. Pat. No. 8,666,080; 20140056467; 20140049447; 20140050326; 20140050334; 20140037117; 20140029773; U.S. Pat. Nos. 8,638,946; 8,638,959; 20140025287; 20140023217; 20140023211; 20140019037; 20140016788; 20140016801; 20140010391; 20140011819; U.S. Pat. No. 8,627,213; 20130343550; 20130342731; U.S. Pat. No. 8,611,186; 20130322669; 20130322667; 20130314610; 20130315401; 20130314599; 20130315422; 20130294605; 20130289754; 20130282388; 20130279723; 20130272527; 20130272536; 20130275128; 20130268281; 20130243200; 20130243226; 20130236040; 20130216048; 20130216073; 20130208909; 20130208897; 20130208898; 20130208899; 20130208900; 20130208926; 20130202116; 20130202114; 20130202117; 20130202125; 20130194107; 20130195297; 20130182064; 20130182853; 20130177166; 20130177187; 20130178967; 20130170679; 20130169779; 20130170649; 20130170680; 20130163766; 20130162904; 20130163765; U.S. Pat. No. 8,473,083; 20130158000; 20130158856; 20130156220; 20130154930; 20130148813; U.S. Pat. No. 8,462,970; 20130141221; 20130144922; 20130142338; 20130121515; 20130121516; 20130121505; U.S. Pat. No. 8,442,244; 20130114821; 20130106997; 20130108096; U.S. Pat. No. 8,428,269; 20130094683; 20130089215; 20130089225; 20130077792; 20130063550; 20130064375; U.S. Pat. No. 8,396,282; 20130051566; 20130051565; 20130051563; 20130046790; 20130045986; 20130044884; 20130041648; 20130036452; 20130035935; 20130028424; 20130022222; 20130016130; 20130010970; 20130010969; 20130003998; 20120328107; 20120314876; 20120314872; 20120307048; U.S. Pat. No. 8,320,591; 20120294446; 20120288126; 20120289496; 20120288124; 20120283593; 20120268563; 20120263311; 20120259442; 20120259643; 20120257764; 20120243689; 20120243695; 20120237037; 20120232912; 20120213375; 20120207310; 20120207308; 20120201389; 20120201405; U.S. Pat. No. 8,229,134; 20120183161; 20120177204; 20120170757; 20120162362; 20120155679; 20120155681; 20120155680; 20120150542; 20120133643; 20120133734; 20120128161; 20120128179; 20120128160; 20120128166; 20120128174; 20120121093; 20120121092; 20120124470; 20120117502; 20120109645; 20120105603; 20120101610; 20120099733; 20120093329; 20120092566; 20120093320; 20120093348; 20120076305; 20120078398; 20120078399; 20120070021; 20120070005; 20120069711; 20120063610; 20120057732; 20120051568; 20120039477; U.S. Pat. No. 8,116,500; 20120022842; 20120020480; 20120020502; 20120016640; 20120014527; 20120008789; U.S. Pat. No. 8,094,834; 20120002024; 20110316996; 20110316967; 20110316966; U.S. Pat. No. 8,085,958; 20110305344; 20110305345; 20110301729; 20110299707; 20110293108; 20110293129; 20110274278; 20110267440; 20110268281; 20110264456; 20110264450; 20110261966; 20110249758; 20110249819; 20110243357; 20110243338; 20110243336; 20110222707; 20110216906; 20110216925; 20110216926; U.S. Pat. Nos. 8,014,532; 8,014,541; 20110211702; 20110208331; 20110206223; 20110206209; 20110200195; 20110194712; 20110188662; 20110188660; 20110182432; 20110170721; U.S. Pat. Nos. 7,974,847; 7,974,418; 20110158427; 20110158413; 20110152260; 20110150098; 20110150257; 20110153043; 20110135098; 20110137437; 20110128821; 20110129105; 20110118257; 20110116638; U.S. Pat. No. 7,945,856; 20110109798; 20110109722; 20110103591; 20110106825; 20110091044; 20110075850; 20110075857; 20110064230; 20110064243; 20110051940; 20110046761; 20110038485; 20110026745; 20110021513; 20110009771; 20100329490; 20100329466; 20100303267; 20100303246; 20100303266; 20100296678; U.S. Pat. No. 7,840,668; 20100290636; U.S. Pat. No. 7,835,535; 20100284549; 20100284551; 20100266112; 20100266133; 20100262419; 20100246832; 20100246831; 20100241256; 20100241438; 20100232621; 20100235169; 20100215164; 20100215199; 20100217586; 20100215044; 20100202632; 20100202629; 20100202620; 20100198585; 20100198589; 20100191537; 20100182394; 20100183159; 20100177910; 20100177906; 20100172508; 20100166238; 20100169781; 20100162119; 20100145711; 20100128880; 20100125453; U.S. Pat. Nos. 7,720,212; 7,715,575; 20100092016; 20100094443; 20100080396; 20100053210; 20100046767; 20100040238; 20100040240; 20100027799; 20100023637; 20100017195; 20100008528; 20100002886; U.S. Pat. No. 7,640,282; 20090318826; 20090312819; 20090313028; 20090304203; 20090304189; 20090304188; 20090304186; 20090304214; 20090296944; 20090296949; 20090292544; 20090290739; 20090285420; 20090287324; 20090264114; 20090252356; 20090238372; 20090238370; 20090238371; U.S. Pat. No. 7,593,539; 20090232317; 20090226002; 20090214045; 20090210238; 20090208022; 20090208027; 20090198753; 20090189830; 20090189974; 20090185693; 20090182563; 20090180625; 20090177479; 20090172060; 20090169037; 20090161879; 20090150163; 20090150161; 20090144063; 20090136063; 20090136044; 20090136057; 20090136047; 20090136048; 20090136066; 20090128617; 20090129601; 20090122994; 20090122995; 20090122648; 20090116652; 20090116657; 20090110203; 20090103738; 20090103753; 20090103737; 20090097663; 20090097359; 20090086982; 20090080632; 20090074197; U.S. Pat. No. 7,505,601; 20090067636; 20090052703; 20090046865; 20090046864; 20090046871; 20090041254; 20090043591; 20090041271; 20090037177; 20090030552; U.S. Pat. No. 7,482,951; 20090022343; 20090012796; 20090010441; 20090007596; 20090010455; 20080319765; 20080306720; 20080304670; 20080306745; 20080292121; 20080279388; 20080279401; 20080275711; 20080273721; 20080273708; 20080268470; 20080262854; 20080260174; 20080253578; 20080247556; 20080240458; 20080243278; 20080235006; 20080228501; 20080226103; 20080226084; 20080219485; 20080221907; 20080212784; 20080212809; 20080215239; 20080205658; 20080205660; 20080205675; 20080199023; 20080192941; 20080187156; 20080187143; 20080178088; U.S. Pat. No. 7,403,625; 20080170703; 20080159571; 20080159507; 20080159544; 20080152152; U.S. Pat. No. 7,391,877; 20080144864; 20080144794; 20080137870; 20080140426; 20080130904; 20080130408; 20080130906; U.S. Pat. Nos. 7,382,885; 7,378,963; 20080118078; 20080114606; 20080107287; U.S. Pat. Nos. 7,369,665; 7,369,668; 20080103615; 20080089523; 20080070685; 20080063224; 20080063211; 20080056517; 20080049948; 20080046246; 20080037809; 20080037795; 20080037796; 20080031475; 20080031462; 20080033729; 20080033730; 20080025534; 20080025519; 20080015463; 20080013762; 20080008341; 20080008324; 20080008327; 20080008342; 20080004866; 20070291967; 20070291950; 20070286427; 20070286426; 20070280485; 20070272022; 20070269061; 20070269064; 20070270988; 20070263985; 20070263823; 20070233466; 20070230729; 20070230725; 20070223750; 20070223708; 20070223749; 20070217590; 20070217639; 20070211904; 20070213990; 20070195964; U.S. Pat. No. 7,260,231; 20070189559; 20070179781; 20070172086; 20070160216; 20070162278; 20070160242; 20070160217; 20070160219; 20070160222; 20070154019; 20070140506; 20070133831; U.S. Pat. No. 7,231,054; 20070127730; 20070127750; 20070121956; 20070121951; 20070109977; 20070098198; 20070092084; 20070074621; 20070076892; 20070070069; 20070071247; U.S. Pat. No. 7,197,151; 20070063822; 20070061026; 20070050212; U.S. Pat. No. 7,180,997; 20070037134; U.S. Pat. No. 7,174,229; 20070025538; 20070019812; 20070021961; U.S. Pat. No. 7,167,567; 20070009120; 20070005348; 20060291680; 20060274901; 20060277034; 20060251278; U.S. Pat. No. 7,133,730; 20060245305; 20060247918; 20060241808; 20060238877; 20060236159; 20060222182; U.S. Pat. Nos. 7,116,788; 7,113,609; 7,113,610; 20060212147; 20060198542; 20060198527; 20060177078; 20060173690; U.S. Pat. No. 7,085,393; 20060153408; 20060147068; 20060142667; U.S. Pat. No. 7,068,792; 20060133628; U.S. Pat. Nos. 7,065,498; 7,058,168; 20060116886; 20060115100; 20060116930; 20060115091; 20060109986; 20060104458; 20060093997; U.S. Pat. No. 7,039,477; 20060083394; 20060083385; 20060085200; U.S. Pat. No. 7,031,474; 20060078129; 20060072764; U.S. Pat. No. 7,024,259; 20060062409; 20060062410; 20060056638; 20060056639; 20060050916; 20060050908; 20060050890; 20060050909; 20060045274; 20060045294; 20060045276; 20060047519; 20060045295; U.S. Pat. No. 7,006,638; 20060029912; U.S. Pat. No. 6,996,244; 20060018497; U.S. Pat. No. 6,990,205; 20060013409; U.S. Pat. No. 6,987,856; 20060008100; 20060001532; 20050280519; 20050281408; 20050276419; 20050276430; 20050276420; 20050271212; 20050270146; 20050271214; 20050273324; 20050271213; U.S. Pat. No. 6,973,192; 20050265558; 20050264857; 20050259832; 20050254660; 20050249356; 20050249359; 20050238176; U.S. Pat. No. 6,956,955; 20050213528; 20050213786; 20050213777; 20050201576; 20050180579; U.S. Pat. Nos. 6,931,370; 6,931,123; 20050175204; 20050163330; 20050147261; 20050142032; 20050143384; 20050135643; 20050135629; 20050129249; 20050124667; 20050124664; 20050117771; 20050117762; 20050100171; 20050089181; 20050084117; 20050078833; 20050080616; 20050069140; 20050069143; 20050063551; 20050058304; 20050053249; U.S. Pat. No. 6,862,356; 20050041816; 20050019734; 20050018039; U.S. Pat. Nos. 6,845,338; 6,839,438; 20040252852; 20040252849; 20040247134; 20040247144; 20040240681; 20040240682; 20040228215; 20040218771; 20040202338; 20040196991; 20040196982; 20040196994; 20040190700; U.S. Pat. No. 6,795,556; 20040175008; 20040175005; U.S. Pat. No. 6,768,798; 20040141619; 20040141622; U.S. Pat. No. 6,766,028; 20040136538; 20040136541; 20040138874; U.S. Pat. No. 6,760,447; 20040111171; 20040109570; 20040105559; 20040105550; U.S. Pat. Nos. 6,741,706; 6,741,711; 6,738,479; 20040091119; U.S. Pat. No. 6,735,564; 20040076306; 20040076301; U.S. Pat. No. 6,718,042; 20040062401; 20040055447; 20040056762; U.S. Pat. No. 6,700,980; 20040039464; U.S. Pat. No. 6,697,491; 20040029086; 20040013278; 20040013252; 20040013271; 20030227476; 20030223602; 20030219130; U.S. Pat. Nos. 6,643,375; 6,643,744; 20030202665; U.S. Pat. Nos. 6,639,989; 6,633,648; 20030190047; 20030185403; 20030172097; U.S. Pat. No. 6,614,910; 20030161478; U.S. Pat. No. 6,606,689; 20030142830; 20030139851; 20030138116; 20030138107; U.S. Pat. No. 6,590,983; 20030123676; 20030118192; 20030108216; U.S. Pat. Nos. 6,577,736; 6,574,339; 20030098892; 20030099369; 20030095668; 20030095669; 20030081115; 20030076973; 20030063574; 20030059070; 20030059057; 20030053680; 20030044002; 20030035553; 20030031333; 20030026441; 20030021423; 20030021429; 20030016837; 20030018477; U.S. Pat. No. 6,507,658; 20030007648; U.S. Pat. Nos. 6,504,933; 6,504,934; 20030002693; 20020196947; U.S. Pat. No. 6,498,857; 20020164037; 20020161808; 20020154179; 20020156633; U.S. Pat. No. 6,470,087; 20020151997; 20020150254; 20020151996; 20020150257; 20020150256; U.S. Pat. Nos. 6,466,913; 6,466,250; 20020147586; 20020143414; 20020141597; 20020133327; 20020129151; U.S. Pat. Nos. 6,449,368; 6,442,277; 20020111705; U.S. Pat. Nos. 6,424,719; 6,421,446; 20020080977; U.S. Pat. Nos. 6,402,782; 6,389,033; 20020055827; U.S. Pat. Nos. 6,385,320; 6,370,256; 20020039421; U.S. Pat. No. 6,363,155; 20020034307; 20020034308; 20020021811; U.S. Pat. No. 6,343,131; 20020006206; 20010040968; U.S. Pat. No. 6,307,941; 20010031051; 20010031057; 20010031053; 20010012370; U.S. Pat. Nos. 6,259,957; 6,259,795; 6,243,476; 6,223,090; 6,215,879; 6,185,152; 6,181,800; 6,178,250; 6,178,245; 6,175,631; 6,173,061; 6,144,747; 6,118,875; 6,091,894; 6,083,163; 6,078,669; 6,072,877; 6,067,361; 6,055,619; 6,038,330; 6,011,851; 6,009,178; 6,002,775; 5,997,439; 5,982,903; 5,979,586; 5,974,152; 5,946,400; 5,928,311; 5,917,916; 5,912,976; 5,910,990; 5,889,867; 5,883,961; 5,862,227; 5,850,453; 5,841,879; 5,835,895; 5,825,894; 5,809,149; 5,798,922; 5,785,661; 5,761,315; 5,751,815; 5,751,817; 5,742,689; 5,729,612; 5,715,317; 5,711,308; 5,661,812; 5,659,619; 5,645,074; 5,633,369; 5,622,172; 5,598,478; 5,596,644; 5,579,396; 5,521,981; 5,500,900; 5,495,576; 5,490,937; 5,459,790; 5,438,623; 5,404,406; 5,325,436; 5,322,841; 5,272,757; 5,187,692; and 5,173,944.

Additionally, the control unit can dynamically adjust the spatial audio cues in response to data from the measurement device. If the sensors detect that the subject is transitioning between sleep stages, the control unit can modify the spatial audio to support this transition, helping the subject to smoothly enter or exit different sleep states.

The auditory stimuli can include a range of sounds, such as but not limited to binaural beats, rhythmic tones, spatial audio cues, natural ambient noises, white noise, pink noise, pulsating sounds, gentle music, spoken instructions, and simulated environmental sounds.

In an embodiment, the auditory stimuli include binaural beats. Binaural beats are usually an auditory illusion perceived when two different pure-tone sine waves, both with frequencies lower than 1500 Hz and less than 40 Hz difference between them, are presented to a subject dichotically (one through each ear).

The control unit is preferably designed to modulate these binaural beats based on the detected sleep state. The measuring device collects data on the subject's physiological parameters to determine the subject's current sleep stage.

Based on the detected sleep state, the control unit can adjust the frequency difference between the tones delivered to each ear. For example, during light sleep or periods of relaxation before falling asleep, the control unit might set the frequency difference to promote alpha brain wave activity, which is associated with relaxation and calmness. As the subject transitions into deeper sleep stages, the control unit can adjust the frequency difference to promote delta brain wave activity, which is associated with deep, restorative sleep.

The modulation of binaural beats allows for a dynamic and adaptive sleep induction process. By continuously adjusting the auditory stimuli to match the subject's current physiological state, the control unit ensures that the binaural beats remain effective and appropriate throughout the entire sleep cycle. This personalized approach helps the subject achieve and maintain desired sleep states more efficiently, enhancing overall sleep quality.

The binaural beats may comprise tones with slightly different frequencies presented to each ear. For example, if a tone with a frequency of 300 Hz is played in the left ear and a tone with a frequency of 310 Hz is played in the right ear, the subject perceives a third tone that is the difference between the two frequencies, in this case, 10 Hz. This perceived third tone creates a beating effect, known as a binaural beat. The control unit utilizes this phenomenon to influence the subject's brain wave activity, promoting relaxation, focus, or different sleep stages depending on the specific frequencies used.

In another embodiment, the auditory stimuli delivered by the control unit include rhythmic tones and/or beats. These rhythmic tones are designed to create a perception of movement, which can be highly effective in promoting relaxation and facilitating sleep.

The control unit can be equipped with the capability to adjust these rhythmic tones based on the monitored sleep state of the subject.

Based on the detected sleep state, the control unit can dynamically adjust the characteristics of the rhythmic tones, including their tempo, volume, and pattern. For example, during the initial stages of sleep or when the subject is experiencing light sleep or no sleep at all, the control unit might deliver slower, softer rhythmic tones to promote deeper relaxation and help the subject transition into sleep or deeper sleep stages. Conversely, if the subject is in a deep sleep stage, the control unit might maintain a steady, gentle rhythm to support and prolong this restful state.

The rhythmic tones and/or beats create an auditory environment that mimics the sensation of movement, which can be particularly soothing and conducive to sleep. The perception of gentle, rhythmic motion, such as rocking or swaying, can help synchronize the subject's brain waves and encourage a state of relaxation, making it easier to fall asleep and maintain sleep throughout the night.

In another embodiment, the auditory stimuli include spatial audio designed to position sounds around the subject. This spatial audio technology creates an immersive sound environment by simulating the way sounds arrive at the ears from different directions and distances, thereby enhancing the perception of a three-dimensional auditory space.

The control unit is preferably equipped with the capability to adapt the spatial audio based on the assessed sleep state of the subject.

Based on the detected sleep state, the control unit can dynamically adjust the spatial audio characteristics to optimize the auditory environment. For example, during the initial stages of sleep or light sleep, the control unit might position sounds to create a calming and enveloping atmosphere that encourages relaxation and helps the subject transition into deeper sleep stages. During deeper sleep stages, the control unit might minimize abrupt spatial changes and maintain a consistent and gentle sound positioning to avoid disruptions and support prolonged restfulness.

The spatial audio cues can simulate natural environments, such as the gentle rustling of leaves, the sound of waves lapping on the shore, or the rhythmic hum of a train, all positioned to create a realistic and soothing auditory landscape. This immersive experience can enhance the subject's sense of comfort and security. Furthermore, these spatial audio stimuli preferably induce a perception of movement, such as the sensation of rocking or swaying, which is known to promote relaxation and facilitate sleep.

In another embodiment, the auditory stimuli contain artificial spatial cues designed to improve the perception of spatial sounds. The aim of these artificial spatial cues is to enhance the subject's perception of movement, such as the sensation of rocking or swaying, which can promote relaxation and facilitate sleep.

The control unit may utilize audio processing techniques to generate these artificial spatial cues. These cues manipulate the timing, volume, and frequency characteristics of sounds to create the illusion that the sounds are originating from specific locations and moving through the three-dimensional space around the subject. This creates an immersive and realistic auditory experience.

Based on the detected sleep state, the control unit can dynamically adjust the artificial spatial cues to optimize the perception of movement. For example, during the initial stages of sleep or light sleep, the control unit might create subtle, slow-moving sound cues that mimic the sensation of gentle rocking or swaying, helping the subject relax and transition into deeper sleep stages. During deeper sleep stages, the control unit might maintain a steady and consistent spatial audio environment to support prolonged restfulness without causing disruptions.

The use of artificial spatial cues can simulate various environments and movements, such as the sensation of being on a boat with the sound of waves gently moving around, or the feeling of being in a moving vehicle with the rhythmic hum of the engine and passing surroundings. These auditory illusions help to synchronize the subject's brain waves with the perceived movement, encouraging a state of relaxation and enhancing the overall sleep experience.

In another embodiment, the auditory stimuli include natural ambient noises, such as the sound of ocean waves, rustling leaves, rain, or other soothing environmental sounds. These natural ambient noises are designed to create a calming and serene auditory environment that promotes relaxation and facilitates sleep. Additionally, these sounds are capable of inducing a perception of gentle movement, which can further enhance the subject's relaxation and aid in sleep induction.

The control unit is preferably capable of selecting and modulating these natural ambient noises based on the subject's detected sleep state.

Based on the detected sleep state, the control unit can dynamically adjust the characteristics of the natural ambient noises. For instance, during the initial stages of sleep or periods of light sleep, the control unit might deliver the gentle sound of ocean waves or the rustling of leaves to help the subject relax and transition into deeper sleep stages. These natural sounds can create an auditory environment that mimics the gentle, rhythmic motion of rocking or swaying, such as the consistent lapping of waves on the shore, which can induce a perception of movement.

During deeper sleep stages, the control unit can maintain a consistent and soothing sound environment to support uninterrupted and restful sleep. The repetitive and rhythmic nature of these sounds helps to reinforce the sensation of gentle movement, promoting a deeper state of relaxation and enhancing the overall sleep experience.

These natural ambient noises can mimic the calming effects of nature, which are known to reduce stress and anxiety. The sounds of ocean waves, for example, can provide a rhythmic and repetitive auditory pattern that is inherently soothing, while the rustling of leaves can create a gentle and tranquil atmosphere. By using these sounds, the device can help the subject achieve a state of relaxation more effectively, making it easier to fall asleep and stay asleep. The perception of gentle movement induced by these natural sounds further aids in synchronizing the subject's brain waves with the calming stimuli, promoting deeper and more restorative sleep.

In another embodiment, the auditory stimuli include white noise and/or pink noise. These types of noise are effective in masking disruptive background sounds and can help in maintaining a stable sleep environment. The control unit can modulate the intensity and characteristics of these noises based on the detected sleep state.

In another embodiment, the auditory stimuli include pulsating sounds, which may have intermittent variations in volume and/or frequency. These pulsating sounds can be used to promote relaxation or to gently stimulate the subject during lighter sleep stages. The control unit adjusts the pulsation patterns based on the real-time sleep data to optimize the sleep-inducing effect.

In another embodiment, the auditory stimuli include spoken instructions or soothing words. These can be pre-recorded messages or dynamically generated content designed to guide the subject into a relaxed state. The control unit can select and modulate these spoken stimuli based on the subject's current sleep state, ensuring they are effective in promoting sleep and relaxation.

A second aspect relates to a method for inducing sleep in a subject using a device comprising first and second headphones, a control unit operatively connected to the first and second headphones, wherein the control unit is adapted to deliver the stimuli to the first and second audio transducers; and adapted to detect a sleep state of the subject, wherein the measuring device is operatively connected to the control unit, whereas the method comprises delivering auditory stimuli by the control unit to induce a sensation of motion, detecting a sleep state of the subject using the measuring device and adapting the auditory stimuli based on the detected sleep state.

This method allows for a dynamic and personalized adjustment of the auditory stimuli, based on the subject's physiological data. This promotes an efficient transition through various sleep stages, improves sleep quality, and ensures sustained restfulness.

The device used in the method is preferably configured as described above.

In an embodiment of the method, if no sleep state is detected, the control unit repeats or continues delivering auditory stimuli to maintain the sensation of motion. This continuous delivery ensures that the subject remains in a relaxed and calm state, which is conducive to falling asleep. The control unit preferably monitors the information from the measuring device and, in the absence of a detected sleep state, persistently provides the auditory stimuli to enhance the subject's relaxation and facilitate the transition into sleep. This embodiment leverages the ongoing sensation of motion to promote a smooth and effective sleep induction process.

In an embodiment of the method, the auditory stimuli are modulated and/or selected based on the sleep stage detected by the measuring device. The control unit receives information from the measuring device and adjusts the characteristics of the auditory stimuli accordingly. For instance, during light sleep or periods of wakefulness, the control unit may deliver soothing, rhythmic tones or binaural beats to promote relaxation and transition into deeper sleep stages. As the subject moves into deeper stages of sleep, the control unit may reduce the intensity of the stimuli or switch to more stable and calming sounds to maintain the restful state. This dynamic modulation and selection of auditory stimuli ensure that the sleep environment is continuously optimized, enhancing the effectiveness of the sleep induction process and the overall sleep quality of the subject.

In an embodiment of the method, modulating the auditory stimuli comprises adjusting the volume, frequency, and/or type of auditory stimuli. The control unit receives information from the measuring device and dynamically alters these characteristics to match the detected sleep stage of the subject. For example, during light sleep or periods of wakefulness, the control unit may increase the volume and frequency of the stimuli to encourage relaxation and transition into deeper sleep stages. As the subject moves into deeper sleep stages, the control unit may decrease the volume and frequency or switch to more soothing types of auditory stimuli, such as gentle nature sounds or soft white noise, to maintain a restful state. This precise modulation of the auditory stimuli ensures a personalized and effective sleep induction process, enhancing the subject's overall sleep quality.

Further advantages and aspects of the invention will become apparent from the claims and from the following description of preferred embodiments, which are explained below with reference to the figures.

The system may employ vestibular augmentation to create a perception of rocking or rolling. Head-related transfer functions (HRTF's), and real-time signal and sensor processing can be used to project a moving auditory object relative to a subject. Synthetic Doppler effects and acoustic parallax can be used to induce or enhance linear vection components.

A head-related transfer function (HRTF) characterizes how an ear receives a sound from a point in space. As sound strikes the subject, the size and shape of the head, ears, and ear canal, the density of the head, and the size and shape of nasal and oral cavities, all transform the sound and affect how it is perceived, boosting some frequencies and attenuating others. The HRTF describes how a sound from a specific point in space arrives at the ear (generally at the outer end of the auditory canal) and is heard by the subject. Thus, a pair of HRTFs for two ears can be used to synthesize a binaural sound that seems to come from a particular point in space. Although humans have only two ears, sounds can be located in three dimensions (range or distance and direction above and below, in front and to the rear, and to either side) because the brain, inner ear and the external ears (pinna) work together to make inferences about location. An HRTF can be “generic” and used as a heuristic for acceptable spatial audio, or personalized, representing the actual listener's head.

Humans estimate the location of a source by taking cues derived from one ear (monaural cues), and by comparing cues received at both ears (difference cues or binaural cues). Among the difference cues are time differences of arrival and intensity differences. The monaural cues come from the interaction between the sound source and the human anatomy, in which the original source sound is modified before it enters the ear canal for processing by the auditory system. These modifications encode the source location, and may be captured via an impulse response which relates the source location and the ear location. This impulse response is termed the head-related impulse response (HRIR). Convolution of an arbitrary source sound with the HRIR converts the sound to that which would have been heard by the listener if it had been played at the source location, with the listener's ear at the receiver location. The HRTF is the Fourier transform of the HRIR. Thus, the HRTF from a given source location can be obtained by measuring the corresponding HRIR, h(t), at the ear drum for an impulse function Δ(t) placed at the source location (e.g., using one or more sensors), and taking the Fourier transform of the HRIR. The measurements can be repeated to obtain an array of HRTFs corresponding to many different points in space around the subject.

Using the HRTF, a “virtual auditory space” surrounding a subject wearing headphones can be created based on the assumption that if the acoustical waveforms present at the subject's eardrums are the same whether coming from the headphones or from free space, then the subject's listening experience should be the same. Generally, sounds generated from headphones appear to originate from inside the head. Using the HRTF, the headphones can instead spatially position the sound in the virtual auditory space surrounding the subject's head. By filtering a sound by the HRTF corresponding to a specific location in space (e.g., directly behind the subject, to the right of the subject, or directly below the subject), the sound can be made to appear to the subject as though it were coming from that location in space, even though in reality, the sound is produced through the headphones worn by the subject. The sound may also come from an array of acoustic transducers (speakers), controlled to produce sounds at the listener's ears according to the HRTF.

A specific HRTF is unique to each person, due to its dependence on physical characteristics of the person (e.g., size and shape of parts of the ears, etc.); however, a generalized HRTF can be used with acceptable accuracy to create the virtual auditory space for most users. For example, the HRIRs for a “dummy head” of idealized geometry can be measured, and the corresponding HRTFs calculated to produce an array of generalized HRTFs. These generalized HRTFs can be used to filter sounds so as to position the sounds at specific points in the virtual auditory space around a user. This approach may be significantly less expensive and more versatile than creating an array of user-specific HRTFs for individual operators.

Auditorily induced vection (AIV) may be achieved by using the HRTFs to position a certain sound or sounds at a specific perceived location relative to a user, and by controlling characteristics of the sound(s), such as volume, for example. The AIV can be applied to enhance the user's attitude awareness, for example by providing a sense of direction or directional motion, such as a perceived sense of gravity.

In some cases, the subject is not stationary during the process, and therefore real vestibular stimulation may be present. This may be detected using inertial sensors such as accelerometers, gyroscopes, magnetometer direction sensors, etc. Alternately, other sensors (e.g., video), either local to or in the vicinity of the subject may be used. The inertial sensors may be present in a headset, and/or in a stationary frame.

AIV may be used to provide a person with a sense of motion that offsets the vehicle's motion, preventing or alleviating motion sickness.

Audio signals may be Doppler shifted for consistency with the intended motion.

A haptic user interface may be employed to enhance the vection.

In various embodiments, the controller may include one or more processors, specialized processors, or microcontrollers. The controller may include specially-programmed, special-purpose hardware, for example, an application-specific integrated circuit, or more generally designed hardware, such as a field programmable gate array (FPGA) or a processor. In one embodiment, the controller is connected to the memory device, e.g., a flash memory device, or other device for storing data. The processor may be one or more ARM RISC processors.

The auditory object and the subject's orientation may be provided from the acoustic reference sensors and the attitude sensors as analog signals. The analog signals may be conditioned (e.g., amplified, filtered, attenuated, etc.) before further processing and/or conversion. The analog signals provided from acoustic reference sensors may be sensitive or susceptible to noise and may be conditioned (e.g., amplified) before further processing and/or conversion. The signal conditioning may be performed using devices such as amplifiers, filters, attenuators, etc. The signal conditioning devices may be internal or external to the acoustic reference sensors and the attitude sensors.

The conditioned analog signals may be converted to digital signals representing the auditory object and the subject's orientation. The analog-to-digital conversion may be performed by one or more analog-to-digital converters (ADC). The ADC devices may be internal or external to the sensors. The sensors may also be direct digital or pulse coded sensors. The sensors may communicate with a controller by a wired or wireless communication (e.g., Bluetooth, BLE, WiFi, optical communication).

Digital signals from the controller may be converted to analog signals representing the projected auditory object by a digital-to-analog converter or other digitally-controller output.

It is therefore an object to provide a method for facilitating a sleep state in a subject, comprising: generating a sensory stimulus selected from the group consisting of an auditory stimulus, a visual stimulus, and a vibrational stimulus; presenting the sensory stimulus to the subject via at least one transducer substantially without producing a rhythmic motion of the subject; and dynamically modulating, via a processing module, a characteristic of the sensory stimulus to induce a sensation of rhythmic motion in the subject, thereby facilitating the sleep state.

It is also an object to provide a system for facilitating a sleep state in a subject, comprising: a stimulus generator configured to produce a sensory stimulus selected from the group consisting of an auditory stimulus, a visual stimulus, and a vibrational stimulus; at least one transducer, communicatively coupled to the stimulus generator, configured to present the sensory stimulus to the subject substantially without producing a rhythmic motion of the subject; and a processing module communicatively coupled to the stimulus generator and the at least one transducer, the processing module being configured to dynamically modulate a characteristic of the sensory stimulus to induce a sensation of rhythmic motion in the subject to facilitate the sleep state.

It is a further object to provide a system for facilitating a sleep state in a subject, comprising: a stimulus generator configured to produce a sensory stimulus; at least one transducer configured to present the sensory stimulus to the subject substantially without producing a rhythmic motion of the subject; and a control unit communicatively coupled to the at least one biometric sensor, the stimulus generator, and the at least one transducer, the control unit configured to control the stimulus generator to produce a modulated sensory stimulus that induces a sensation of rhythmic motion in the subject to facilitate the sleep state.

The method may further comprise monitoring at least one biometric parameter of the subject using a sensor; and adjusting the dynamic modulation of the characteristic of the sensory stimulus based at least in part on the monitored at least one biometric parameter.

The at least one biometric parameter mat be indicative of a sleep stage of the subject, and wherein the processing module is configured to analyze the sleep stage and to adjust the dynamic modulation dependent on the sleep stage.

The sensor is selected from the group consisting of an EEG sensor configured to monitor brain waves of the subject; an EOG sensor configured to detect eye movements of the subject indicative of different sleep stages; a pulse oximeter adapted to measure at least one of blood oxygen levels and heart rate variability of the subject; a plethysmographic sensor configured to measure peripheral blood flow; a temperature sensor configured to measure a temperature of the subject; a galvanic skin response, to measure changes in skin conductivity; a blood pressure monitor; a movement sensor configured to detect body movements; a movement sensor configured to detect beathing of the subject; a movement sensor configured to detect position changes of the subject; a pressure sensor configured to detect changes in pressure on a surface where the subject is positioned; at least one sensor configured to determine a position of the subject over time; at least one inertial sensor configured to detect movement of the subject; and a microphone configured to capture beath sounds.

The sensation of rhythmic motion may comprise a sensation selected from the group consisting of rocking, swaying, and cyclic rotation through an arc with a period of between 1 and 5 seconds.

The sensory stimulus may be the auditory stimulus, and wherein dynamically modulating the characteristic comprises adjusting a phase difference or a temporal delay between at least two audio output channels.

The method may further comprise generating binaural beats and delivering the binaural beats to the subject via the at least two audio output channels.

The sensory stimulus may be the vibrational stimulus, and wherein presenting the sensory stimulus comprises delivering a spatially distributed pattern of vibration to the subject via an array of electromechanical transducers.

The sensory stimulus may be the visual stimulus presented within the subject's field of vision, wherein dynamically modulating the characteristic comprises displaying a rhythmic change of a visual pattern to induce a sensation of vection, wherein the sensation of vection corresponds to a gentle rocking motion to aid in the onset or maintenance of sleep.

The at least one transducer may comprise a spatial array of electromechanical transducers integrated into an article selected from the group consisting of a bed, a mattress, a pillow, and a cushion.

The at least one transducer may comprise at least two audio transducers configured to deliver sound to distinct ears of the subject. The at least two audio transducers comprise headphones, audio speakers, a spatialized array of audio transducers, or the like.

The at least one transducer may be a visual display device.

The sensation of rhythmic motion may comprise a sensation of rocking or swaying with a period between 1 and 5 seconds.

The system may further comprise a user interface configured to receive an input to control at least one of an intensity and a periodicity of the sensation of motion.

The processing module may be configured to modulate the sensory stimulus by applying a variable phase shift or a time-varying inter-channel phase difference to an electrical signal driving the at least one transducer.

The processing module may be configured to apply a variable delay to the dynamically modulated characteristic of the sensory stimulus.

The system may further comprise at least one biometric sensor configured to generate a feedback signal corresponding to a physiological state of the subject, wherein the control unit is further configured to receive the feedback signal from the at least one biometric sensor, and in response to the feedback signal, control the stimulus generator.

The at least one biometric sensor may be is selected from the group consisting of an EEG sensor, an EOG sensor, a pulse oximeter, a plethysmographic sensor, a movement sensor, a pressure sensor, an inertial sensor, and a microphone.

The control unit may be further configured to analyze the feedback signal to determine a sleep stage of the subject and adjust the modulated sensory stimulus based on the determined sleep stage.

The sensory stimulus may be an auditory stimulus, and the at least one transducer comprises at least two audio transducers.

The control unit may control the stimulus generator by adjusting a time-varying inter-channel phase difference or a temporal delay between signals sent to the at least two audio transducers, wherein the time-varying inter-channel phase difference or the temporal delay between signals is adapted to stimulate the subject's vestibular system indirectly via auditory perception, leading to the sensation of rhythmic motion that facilitates the sleep state.

The sensory stimulus may be a vibrational stimulus, and the at least one transducer comprises an array of electromechanical transducers.

The sensory stimulus may be a visual stimulus, and the at least one transducer is a visual display configured to present visual content to the subject

The sensation of rhythmic motion may comprise a perception of cyclic movement, rocking, or swaying along an arcuate path.

The system may further comprise a user interface configured to control the at least one of an intensity and a periodicity of the sensation of rhythmic motion.

The stimulus generator may comprise a visual display device, and the control unit is further configured to: generate visual content representing a rhythmic oscillating motion; and control a presentation of the visual content on the visual display device to induce a sensation of physical swaying or rocking in the subject, thereby facilitating sleep.

The stimulus generator may be configured to produce a sensory stimulus comprises a visual stimulus generator configured to produce a sequence of images.

The system may further comprise a motion control module configured to apply a time-varying spatial transformation to the sequence of images, to produce a time-varying spatial transformed sequence of images, which are adapted to induce the sensation of rhythmic motion in the subject; and a display unit configured to present the time-varying spatial transformed sequence of images to the subject, wherein the apparent rhythmic motion is adapted to stimulate the subject's visual-vestibular integration, leading to a sensation of rhythmic swaying that facilitates sleep.

It is also an object to provide a device for inducing a sensation of motion in a subject using auditory or vibrational stimuli to facilitate sleep, comprising: at least two audio or vibrational transducers; and a control unit operatively connected to the at least two audio or vibrational transducers, comprising at least one automated processor, configured to deliver the audio or vibrational stimuli to the at least two audio or vibrational transducers, to induce the sensation of motion (vection) in the subject representing a cyclic angular movement of the subject.

The device according may further comprise a biometric feedback input to the control unit configured to receive biometric information of the subject.

The at least two audio or vibrational transducers may be configured to emit independent sounds or vibrations from each other.

The at least two audio or vibrational transducers may comprise in-ear headphones, over-the-ear headphones, hearing aids, or bone conduction transducers.

The at least two audio or vibrational transducers speakers may operate without contact to the subject.

The biometric feedback input may comprise an EEG sensor configured to monitor brain waves of the subject, an EOG sensor configured to detect eye movements of the subject indicative of different sleep stages, a pulse oximeter adapted to measure blood oxygen levels and/or heart rate variability of the subject, a photoplethysmography configured to monitor cyclic changes in blood in the skin, a temperature sensor configured to measure a temperature of the subject, a movement sensor configured to detect body movements, a movement sensor configured to detect beathing of the subject, a movement sensor configured to detect position changes of the subject, and/or a microphone configured to capture beath sounds.

The control unit may be further configured to: detect at least one of breathing and snoring patterns; analyze sleep stages of the subject selectively in dependence on the captured breath sounds; analyze sleep stages of the subject selectively in dependence on the measured changes in skin conductivity; analyze sleep stages of the subject selectively in dependence on the detected movement of the subject; analyze sleep stages of the subject selectively in dependence on the detected changes in skin impedance of the subject; analyze sleep stages of the subject selectively in dependence on the determined position of the subject over time; analyze sleep stages of the subject selectively in dependence on a blood pressure of the subject.

The biometric feedback input may be further configured to detect a galvanic skin response, to measure changes in skin conductivity.

The device may further comprise at least one inertial sensor configured to detect movement of the subject.

The device may further comprise at least one movement sensor configured to detect movement of the subject.

The device may further comprise at least one sensor configured to determine a position of the subject over time.

The device may further comprise a blood pressure monitor.

The biometric feedback input may receive information from a peripheral blood flow sensor configured to measure peripheral blood flow using plethysmography, and/or a pressure sensor configured to detect changes in pressure on a surface where the subject is positioned.

the biometric feedback input may be connected optically, wirelessly or through a wired connection to the control unit.

The control unit may be configured to generate a non-auditory, non-vibrational stimulus for the subject.

The audio or vibrational stimuli may be modulated in dependence on the biometric feedback input.

The control unit may comprise a portable device such as a smartphone or smartwatch.

The auditory or vibrational stimuli may comprise rhythmic tones, simulated spatial audio cues, binaural beats, and/or isochronic tones.

The cyclic angular movement of the subject may comprise a rocking perception, or a swaying perception.

The audio or vibrational stimuli may comprise at least two infrasonic electromechanical transducers which operate at different frequencies to produce constructive and destructive interference of vibrational waves.

The audio or vibrational stimuli may comprise at least two infrasonic electromechanical transducers which operate at different frequencies to produce constructive and destructive interference of neuronal signals.

The audio or vibrational stimuli may be delivered through bone conduction transducers.

The audio or vibrational stimuli may comprise spatial audio and/or spatial vibrations.

The audio or vibrational stimuli may comprise artificial spatial cues to improve the perception of spatial sounds.

The audio or vibrational stimuli may comprise natural ambient noises.

The audio or vibrational stimuli comprise white noise or pink noise.

The audio or vibrational stimuli may comprise pulsating sounds, intermittent variations in amplitude, intermittent variations in frequency, spoken instructions and/or soothing words.

The control unit may be configured to generate the audio or vibrational stimuli according to a Head-Related Transfer Function (HRTF). The HRTF may comprises an individualized HRTF or a generic HRTF.

The control unit may be configured to generate the audio or vibrational stimuli according to a model of the subject. The model may comprise a model of vibrational transduction from the at least two audio or vibrational transducers to the vestibular organs of the subject.

It is also an object to provide a method for inducing sleep in a subject using a device as described herein, to deliver auditory or vibrational stimuli to the subject by the control unit to induce vection.

The auditory or vibrational stimuli may be adapted based on a determined sleep state.

If a non-sleeping state is detected, the control unit may repeat or continue delivering auditory stimuli to maintain the vection.

The auditory or vibrational stimuli may be adaptively modulated based on a determined sleep stage, e.g., amplitude modulated, frequency modulated, and/or stimulus type modulated.

It is a further object to provide a device for inducing a sensation of motion in a subject, comprising: a plurality of electromechanical transducers configured to receive an electrical signal and produce vibrations corresponding to the electrical signals; and at least one automated processor configured to produce the electrical signals, wherein the vibrations are adapted to induce the sensation of motion in the subject.

It is another object to provide a method for inducing a sensation of motion in a subject, comprising: defining a vibrational stimulation pattern for a subject; generating a set of electrical signals corresponding to the vibrational stimulation pattern; and transducing the set of electrical signals into a vibrations corresponding to the set of electrical signals with a plurality of electromechanical transducers, wherein the vibrations are adapted to induce the sensation of motion in the subject.

The sensation of motion in the subject may be adapted to induce sleep in the subject.

The sensation of motion may comprise a sensation of cyclic rotation through an arc. The cyclic rotation may have a frequency of 0.2 to 1 Hz.

The plurality of electromechanical transducers may comprise an electro-acoustic transducer configured to produce audible emissions, the audible emissions being adapted to induce binaural beats.

It is another object to provide a method for facilitating sleep in a subject, the method comprising: generating an auditory stimulus; delivering the auditory stimulus to the subject via at least two spatially separated audio output channels; and dynamically adjusting a phase difference between the auditory stimulus delivered to a first audio output channel and the auditory stimulus delivered to a second audio output channel, wherein the dynamically adjusted phase difference is configured to induce a sensation of rhythmic motion in the subject, thereby facilitating sleep.

It is a further object to provide a system for facilitating sleep in a subject, the system comprising: an audio generation module configured to produce an auditory signal; a processing module communicatively coupled to the audio generation module, the processing module configured to apply a variable phase shift to the auditory signal to create at least two phase-shifted auditory signals; and at least two audio output transducers communicatively coupled to the processing module, each transducer configured to deliver one of the at least two phase-shifted auditory signals to a distinct ear of the subject, wherein the variable phase shift is configured to induce a sensation of rhythmic motion in the subject to facilitate sleep.

It is a still further object to provide a method for sensory brain stimulation to promote sleep, the method comprising: providing a multi-channel audio system to a user; transmitting an auditory signal through the multi-channel audio system; and modulating a temporal delay between at least two channels of the multi-channel audio system to create a perceived movement of the auditory signal, wherein the perceived movement mimics a physical rocking motion to induce a sleep state in the user.

It is also an object to provide a device for neurotechnology-assisted sleep induction, the device comprising: a sound generator configured to produce a continuous or intermittent audio waveform; a signal processor configured to receive the audio waveform and apply a time-varying inter-channel phase difference to the audio waveform, thereby creating a first output signal and a second output signal; and a pair of audio transducers, a first transducer configured to receive and emit the first output signal to a first ear of a user, and a second transducer configured to receive and emit the second output signal to a second ear of the user, wherein the time-varying inter-channel phase difference is configured to stimulate the user's vestibular system indirectly via auditory perception, leading to a sensation of rhythmic motion that facilitates sleep.

It is another object to provide a method for facilitating sleep in a subject, the method comprising: generating a dynamic visual stimulus configured to display a rhythmic oscillating motion; presenting the dynamic visual stimulus to the subject's field of vision; and controlling the rhythmic oscillating motion of the dynamic visual stimulus to induce a sensation of physical swaying or rocking in the subject, thereby promoting a sleep state.

It is a further object to provide a system for facilitating sleep in a subject, the system comprising: a visual display device configured to present visual content to the subject; a processing module communicatively coupled to the visual display device, the processing module configured to: generate visual content depicting a rhythmic oscillating motion; and control the presentation of the visual content on the visual display device to induce a sensation of physical swaying or rocking in the subject, thereby facilitating sleep.

It is a still further object to provide a method for sensory brain stimulation to promote sleep, the method comprising: displaying a visual pattern that undergoes continuous rhythmic displacement across a visual field of a user; and adjusting a characteristic of the continuous rhythmic displacement to elicit a visually induced self-motion (vection) sensation in the user, wherein the vection sensation mimics a gentle rocking motion to aid in the onset or maintenance of sleep.

Another object provides a device for neurotechnology-assisted sleep induction, the device comprising: a visual stimulus generator configured to produce a sequence of images; a motion control module configured to apply a time-varying spatial transformation to the sequence of images, thereby creating an apparent rhythmic motion within the displayed images; and a display unit configured to present the transformed sequence of images to a user, wherein the apparent rhythmic motion is configured to stimulate the user's visual-vestibular integration, leading to a sensation of rhythmic swaying that facilitates sleep.

### BRIEF DESCRIPTION OF THE DRAWINGS

FIG. 1 shows a device according to an embodiment of the invention.

FIG. 2 shows a device according to another embodiment of the invention.

FIG. 3 shows a device according to another embodiment of the invention.

FIG. 4 shows a device according to another embodiment of the invention.

FIG. 5 shows a flowchart of an embodiment of the method for inducing sleep.

FIG. 6 shows a flowchart of another embodiment of the method for inducing sleep.

### DETAILED DESCRIPTION OF THE PREFERRED EMBODIMENTS

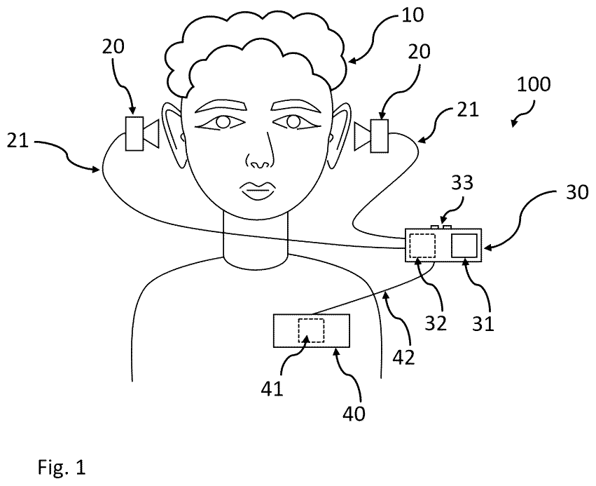

FIG. 1 shows a device 100 in a frontal view of a subject's head 10. In this embodiment, the subject 10 wears wired headphones 20 designed as over-ear headphones. The headphones 20 are operatively connected to the control unit 30 via cables 21, 22. The control unit 30 includes a touch display 31, a battery 32, and two volume buttons 33 for user interface and control.

The control unit 30 is connected to a measuring device 40 via a wire 42. The measuring device 40 is operatively connected to the control unit 30. In this embodiment, the measuring device comprises a heart rate sensor 41 and processes signals from the heart rate sensor 41 to interpret the subject's sleep state. The heart rate sensor 41 continuously monitors the subject's heart rate and computes the data to determine the current sleep state, which is then transmitted to the control unit 30.

As long as the measuring device 40 does not detect any sleep state, the control unit 30 delivers binaural beats to the headphones 20. These binaural beats create a sensation of motion in the subject 10 and facilitate the transition into sleep. Once the measuring device 40 detects that the subject 10 has entered a sleep state, the control unit 30 can adjust or cease the auditory stimuli based on the real-time feedback to optimize the subject's sleep environment.

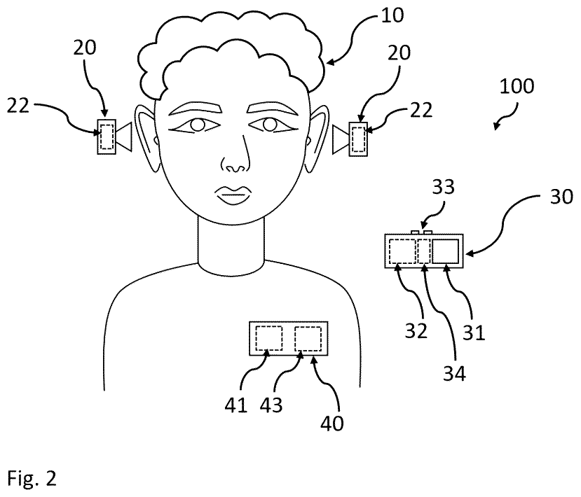

FIG. 2 shows a device 100 in a frontal view of a subject's head, similar to FIG. 1. In this embodiment, the subject 10 wears over-ear headphones 20. However, unlike the previous embodiment, the connections between the headphones 20, the measuring device 40, and the control unit 30 are wireless.

The control unit 30 includes a display 31, a battery 32, and two volume buttons 33 for user interface and control. The control unit 30 is wirelessly connected to the headphones 20, eliminating the need for cables 21.

Additionally, the measuring device 40, which is responsible for processing signals from a heart rate sensor 41 to interpret the subject's sleep state, is also wirelessly connected to the control unit 30. The heart rate sensor 41 continuously monitors the subject's heart rate and computes the data to determine the current sleep state, which is then transmitted wirelessly to the control unit 30.

As long as the measuring device 40 does not detect any sleep state, the control unit 30 delivers binaural beats to the headphones 20. These binaural beats are designed to promote relaxation and facilitate the transition into sleep. Once the measuring device 40 detects that the subject has entered a sleep state, the control unit 30 can adjust or cease the auditory stimuli based on the real-time feedback to optimize the subject's sleep environment.

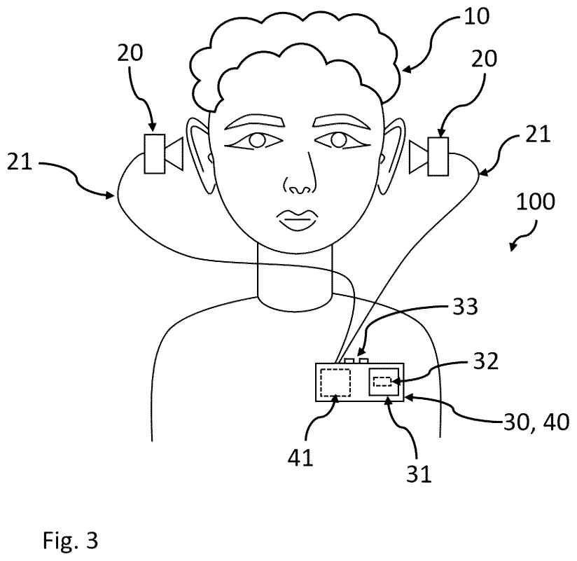

FIG. 3 shows a device 100 in a frontal view of a subject's head, similar to FIGS. 1 and 2. In this embodiment, the subject 10 wears over-ear headphones 20. The headphones 20 are operatively connected to the control unit 30 via cables 21.

In this embodiment, the measuring device 40 is integrated into the control unit 30. The control unit 30 includes a display 31, a battery 32, and two volume buttons 33 for user interface and control. The integrated measuring device 40 processes signals from a heart rate sensor 41 to interpret the subject's sleep state. The heart rate sensor 41 continuously monitors the subject's heart rate and computes the data to determine the current sleep state, which is then used directly by the control unit 30.

As long as the measuring device 40 integrated into the control unit 30 does not detect any sleep state, the control unit 30 delivers binaural beats to the headphones 20. These binaural beats create a sensation of motion in the subject 10 and facilitate the transition into sleep. Once the measuring device 40 detects that the subject has entered a sleep state, the control unit 30 can adjust or cease the auditory stimuli based on the real-time feedback to optimize the subject's sleep environment.

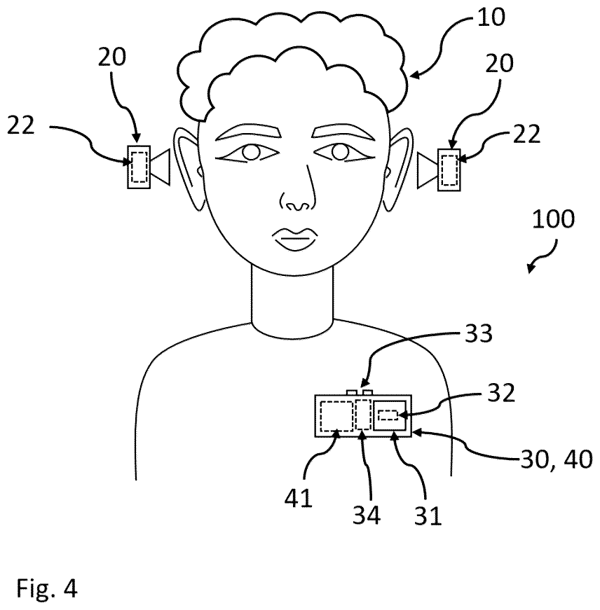

FIG. 4 shows a device 100 in a frontal view of a subject's head, similar to FIG. 3. In this embodiment, the subject 10 wears over-ear headphones 20. The headphones 20 are operatively connected to the control unit 30 wirelessly, utilizing radios 22 in the headphones 20 and radio 34 in the control unit 30.

In this embodiment, the measuring device 40 is integrated into the control unit 30. The control unit 30 includes a display 31, a battery 32, and two volume buttons 33 for user interface and control. The integrated measuring device 40 processes signals from a heart rate sensor 41 to interpret the subject's sleep state. The heart rate sensor 41 continuously monitors the subject's heart rate and computes the data to determine the current sleep state, which is then used directly by the control unit 30.

As long as the measuring device 40 integrated into the control unit 30 does not detect any sleep state, the control unit 30 delivers binaural beats to the headphones 20. These binaural beats create a sensation of motion in the subject 10 and facilitate the transition into sleep. Once the measuring device 40 detects that the subject has entered a sleep state, the control unit 30 can adjust or cease the auditory stimuli based on the real-time feedback to optimize the subject's sleep environment.

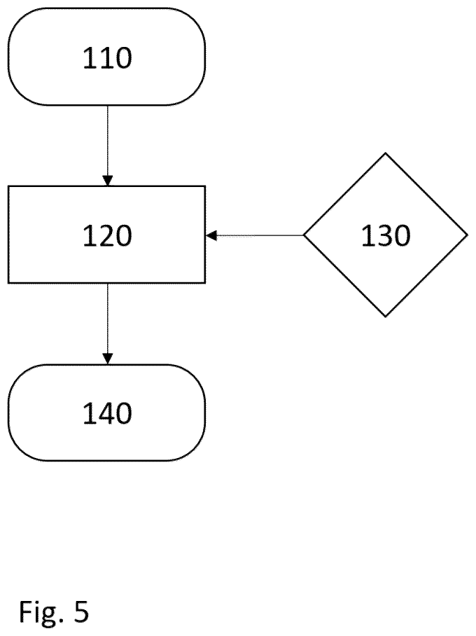

FIG. 5 shows a simple flowchart of the method for inducing sleep using the device 100. The flowchart outlines the steps involved in delivering auditory stimuli to facilitate sleep and adapting these stimuli based on the detected sleep state of the subject.

The method begins at step 110, where the device 100 is powered on and initialized. At step 120, the control unit 30 starts delivering auditory stimuli to the headphones 20. These stimuli are designed to create a sensation of motion and promote relaxation in the subject 10.

At step 130, the measuring device 40 continuously monitors the physiological parameters of the subject 10 to check for a sleep state. This involves processing signals from sensors such as a heart rate sensor 41 to determine if the subject has entered a sleep state. If the measuring device 40 detects a sleep state at decision point 132, it sends feedback to the control unit 30.

Upon receiving feedback that a sleep state has been detected, the control unit 30 stops delivering the auditory stimuli at step 120, resulting in the end position 140. This step ensures that unnecessary stimuli are not delivered once the subject has fallen asleep, thereby maintaining an optimal sleep environment.

This flowchart illustrates the method's basic operational sequence, highlighting the interaction between the control unit 30 and the measuring device 40 to facilitate and optimize the subject's transition into sleep.

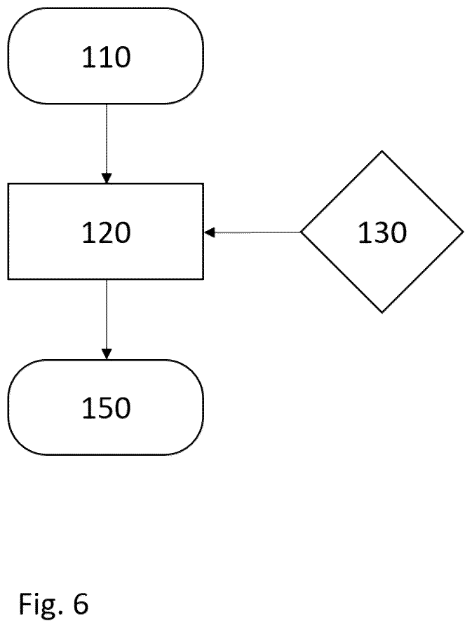

FIG. 6 shows a simple flowchart of the method for inducing sleep using the device 100 in another embodiment. The flowchart outlines the steps involved in delivering and adapting auditory stimuli to facilitate sleep based on the detected sleep state of the subject.

The method begins at step 110, where the device 100 is powered on and initialized. At step 120, the control unit 30 starts delivering auditory stimuli to the headphones 20. These stimuli are designed to create a sensation of motion and promote relaxation in the subject 10.

At step 130, the measuring device 40 continuously monitors the physiological parameters of the subject 10 to detect a sleep state. This involves processing signals from sensors such as a heart rate sensor 41 to determine the current sleep state of the subject.

Once a sleep state is detected at step 130, the control unit 30 alters the stimuli according to the type of sleep state detected. This results in different stimuli being delivered based on the different sleep states identified, as shown in step 150. For instance, during lighter sleep stages, the stimuli may be adjusted to be more rhythmic and intense to promote deeper relaxation, whereas during deeper sleep stages, the stimuli may be softened or transitioned to gentle ambient noises to maintain sleep continuity.

This flowchart illustrates the method's advanced operational sequence, highlighting the real-time adaptation of auditory stimuli by the control unit 30 based on the specific sleep state detected by the measuring device 40. This dynamic adjustment ensures an optimized sleep environment tailored to the subject's current physiological state.

It will be appreciated by those of ordinary skill in the art that the diagrams, schematics, illustrations, and the like represent conceptual views or processes illustrating systems and methods embodying this invention. The functions of the various elements shown in the figures may be provided through the use of dedicated hardware as well as hardware capable of executing associated software.

Although the invention(s) have been described with reference to specific embodiments, it will be understood by those skilled in the art that various changes may be made and equivalents may be substituted for elements thereof without departing from the true spirit and scope of the invention. In addition, modifications may be made without departing from the essential teachings of the invention. The invention is described by way of various embodiments and features. This disclosure is intended to encompass all consistent combinations, subcombinations, and permutations of the different options and features, as if expressly set forth herein individually.

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

The disclosure has been described with reference to various specific embodiments and techniques. However, many variations and modifications are possible while remaining within the scope of the disclosure.

As used herein in this document, the terms “coupled to” and “coupled with” are also used euphemistically to mean “communicatively coupled with” over a network, where two or more devices are able to exchange data with each other over the network, possibly via one or more intermediary device.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### LIST OF REFERENCE SIGNS

10: Subject
20: Headphones
21: Cables (first and second headphones to control unit)
22: Radio units in headphones
30: Control unit
31: Display on control unit
32: Battery in control unit
33: Volume Buttons on control unit
34: Radio unit in control unit
40: Measuring device
41: Heart rate sensor
42: Wire connecting measuring device to control unit
43: Battery in measuring device
44: Radio unit in measuring device
100: Device
110: Start of the method
120: Delivery of stimuli to headphones
130: Detection of sleep state
132: Feedback from measuring device indicating sleep state detected
140: End position of the method
150: Altered stimuli according to sleep state

1. A method for facilitating a sleep state in a subject, comprising:
generating a sensory stimulus selected from the group consisting of an auditory stimulus, a visual stimulus, and a vibrational stimulus;
presenting the sensory stimulus to the subject via at least one transducer substantially without producing a rhythmic motion of the subject; and
dynamically modulating, via a processing module, a characteristic of the sensory stimulus to induce a sensation of rhythmic motion in the subject, thereby facilitating the sleep state.

2. The method of claim 1, further comprising:
monitoring at least one biometric parameter of the subject using a sensor; and
adjusting the dynamic modulation of the characteristic of the sensory stimulus based at least in part on the monitored at least one biometric parameter.

3. The method of claim 2, wherein the at least one biometric parameter is indicative of a sleep stage of the subject, and wherein the processing module is configured to analyze the sleep stage and to adjust the dynamic modulation dependent on the sleep stage.

4. The method of claim 2, wherein the sensor is selected from the group consisting of an EEG sensor configured to monitor brain waves of the subject;
an EOG sensor configured to detect eye movements of the subject indicative of different sleep stages;
a pulse oximeter adapted to measure at least one of blood oxygen levels and heart rate variability of the subject;
a plethysmographic sensor configured to measure peripheral blood flow;
a temperature sensor configured to measure a temperature of the subject;
a galvanic skin response, to measure changes in skin conductivity;
a blood pressure monitor;
a movement sensor configured to detect body movements;
a movement sensor configured to detect beathing of the subject;
a movement sensor configured to detect position changes of the subject;
a pressure sensor configured to detect changes in pressure on a surface where the subject is positioned;
at least one sensor configured to determine a position of the subject over time;
at least one inertial sensor configured to detect movement of the subject; and
a microphone configured to capture beath sounds.

5. The method of claim 1, wherein the sensation of rhythmic motion comprises a sensation selected from the group consisting of rocking, swaying, and cyclic rotation through an arc with a period of between 1 and 5 seconds.

6. The method of claim 1, wherein the sensory stimulus is the auditory stimulus, and wherein dynamically modulating the characteristic comprises adjusting a phase difference or a temporal delay between at least two audio output channels.

7. The method of claim 6, further comprising generating binaural beats and delivering the binaural beats to the subject via the at least two audio output channels.

8. The method of claim 1, wherein the sensory stimulus is the vibrational stimulus, and wherein presenting the sensory stimulus comprises delivering a spatially distributed pattern of vibration to the subject via an array of electromechanical transducers.

9. The method of claim 1, wherein the sensory stimulus is the visual stimulus presented within the subject's field of vision, and wherein dynamically modulating the characteristic comprises displaying a rhythmic change of a visual pattern to induce a sensation of vection, wherein the sensation of vection corresponds to a gentle rocking motion to aid in the onset or maintenance of sleep.

10. A system for facilitating a sleep state in a subject, comprising:
a stimulus generator configured to produce a sensory stimulus selected from the group consisting of an auditory stimulus, a visual stimulus, and a vibrational stimulus;
at least one transducer, communicatively coupled to the stimulus generator, configured to present the sensory stimulus to the subject substantially without producing a rhythmic motion of the subject; and
a processing module communicatively coupled to the stimulus generator and the at least one transducer, the processing module being configured to dynamically modulate a characteristic of the sensory stimulus to induce a sensation of rhythmic motion in the subject to facilitate the sleep state.

11. The system of claim 10, wherein the at least one transducer comprises a spatial array of electromechanical transducers integrated into an article selected from the group consisting of a bed, a mattress, a pillow, and a cushion.

12. The system of claim 10, wherein the at least one transducer comprises at least two audio transducers configured to deliver sound to distinct ears of the subject.

13. The system of claim 10, wherein the at least one transducer comprises a visual display device.

14. The system of claim 10, further comprising a user interface configured to receive an input to control at least one of an intensity and a periodicity of the sensation of motion.

15. The system of claim 10, wherein the processing module is configured to modulate the sensory stimulus by applying a variable phase shift or a time-varying inter-channel phase difference to an electrical signal driving the at least one transducer.

16. The system of claim 10, wherein the processing module is configured to apply a variable delay to the dynamically modulated characteristic of the sensory stimulus.

17. A system for facilitating a sleep state in a subject, comprising:
a stimulus generator configured to produce a sensory stimulus;
at least one transducer configured to present the sensory stimulus to the subject substantially without producing a rhythmic motion of the subject; and
a control unit communicatively coupled to the at least one biometric sensor, the stimulus generator, and the at least one transducer, the control unit configured to control the stimulus generator to produce a modulated sensory stimulus that induces a sensation of rhythmic motion in the subject to facilitate the sleep state.

18. The system of claim 17, further comprising at least one biometric sensor configured to generate a feedback signal corresponding to a physiological state of the subject, wherein the control unit is further configured to receive the feedback signal from the at least one biometric sensor, and in response to the feedback signal, control the stimulus generator.

19. The system of claim 18, wherein the at least one biometric sensor is selected from the group consisting of an EEG sensor, an EOG sensor, a pulse oximeter, a plethysmographic sensor, a movement sensor, a pressure sensor, an inertial sensor, and a microphone, and the control unit is further configured to analyze the feedback signal to determine a sleep stage of the subject and adjust the modulated sensory stimulus based on the determined sleep stage.

20. The system of claim 17, wherein the sensory stimulus is an auditory stimulus, and the at least one transducer comprises at least two audio transducers.

21. The system of claim 17, wherein the control unit controls the stimulus generator by adjusting a time-varying inter-channel phase difference or a temporal delay between signals sent to the at least two audio transducers, wherein the time-varying inter-channel phase difference or the temporal delay between signals is adapted to stimulate the subject's vestibular system indirectly via auditory perception, leading to the sensation of rhythmic motion that facilitates the sleep state.

22. The system of claim 17, wherein the sensory stimulus is a vibrational stimulus, and the at least one transducer comprises an array of electromechanical transducers.

23. The system of claim 17, wherein the sensory stimulus is a visual stimulus, and the at least one transducer is a visual display configured to present visual content to the subject, and
the control unit is further configured to:
generate visual content representing a rhythmic oscillating motion; and
present the visual content on the visual display device to induce a sensation of physical swaying or rocking in the subject, thereby facilitating sleep.

24. The system of claim 17, further comprising a user interface configured to control the at least one of an intensity and a periodicity of the sensation of rhythmic motion.

25. The system of claim 17, wherein the stimulus generator configured to produce a sensory stimulus comprises a visual stimulus generator configured to produce a sequence of images;
further comprising a motion control module configured to apply a time-varying spatial transformation to the sequence of images, to produce a time-varying spatial transformed sequence of images, which are adapted to induce the sensation of rhythmic motion in the subject; and
a display unit configured to present the time-varying spatial transformed sequence of images to the subject, wherein the apparent rhythmic motion is adapted to stimulate the subject's visual-vestibular integration, leading to a sensation of rhythmic swaying that facilitates sleep.
